SEQUENCE LISTING (2 sequences)

SEQ ID NO: 1 - protein, 577 aa, Mus musculus
MSAATQSPMMQMASGNGASDRDPLPPGWEIKIDPQTGWPFFVDHNSRTTTWNDPRVPPEGPKDTASSANGPSRDGSRLLPIREGHPIYPQLRPGYIPIPVLHEGSENRQPHLFHAYSQPGVQRFRTEAAAATPQRSQSPLRGGMTEAAQTDKQCGQMPATATTAAAQPPTAHGPERSQSPAASDCSSSSSSASLPSSGRSSLGSHQLPRGYIPIPVIHEQNITRPAAQPSFHQAQKTHYPAQQGEYQPQQPVYHKIQGDDWEPRPLRAASPFRSPVRGASSREGSPARSGTPVHCPSPIRVHTVVDRPQPMTHREPPPVTQPENKPESKPGPAGPDLPPGHIPIQVIRREADSKPVSQKSPPPAEKVEVKVSSAPIPCPSPSPAPSAVPSPPKNVAAEQKAAPSPAPAEPAAPKSGEAETPPKHPGVLKVEAILEKVQGLEQAVDSFEGKKTDKKYLMIEEYLTKELLALDSVDPEGRADVRQARRDGVRKVQTILEKLEQKAIDVPGQVQVYELQPSNLEAEQPLQEIMGAVVADKDKKGPENKDPQTESQQLEAKAATPPNPSNPADSAGNLVAP

SEQ ID NO: 2 - protein, 575 aa, Homo sapiens
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

(19) United States
(12) Patent Application Publication
(10) Pub. No.: US 2019/0328836 A1
(43) Pub. Date: Oct. 31, 2019

(54) METHODS FOR TREATING ISCHEMIC INJURY

(71) Applicants: Duke University, Durham, NC (US); East Carolina University, Greenville, NC (US)

(72) Inventors: Christopher D. Kontos, Durham, NC (US); Joseph M. McClung, Greenville, NC (US)

(21) Appl. No.: 16/476,791

(22) PCT Filed: Jan. 9, 2018
(86) PCT No.: PCT/US2018/012962

§ 371 Date: Jul. 9, 2019

Related U.S. Application Data
(60) Provisional application No. 62/443,930, filed on Jan. 9, 2017.
(60) Provisional application No. 62/444,167, filed on Jan. 9, 2017.
(60) Provisional application No. 62/448,278, filed on Jan. 19, 2017.

(51) Int. Cl. A61K 38/17; C07K 14/47; A61P 9/10

(52) U.S. Cl. CPC A61K 38/1709; A61P 9/10; C07K 14/4702

(57) ABSTRACT

Embodiments of the present disclosure relate generally to the treatment and prevention of ischemic injury. More particularly, embodiments of the present disclosure include materials and methods for treating and preventing ischemic injury caused by diseases like peripheral artery disease (PAD) by modulating various candidate genes within the Lsq-1 QTL, such as Bcl-2-associated athanogene-3 (Bag3). Given that no alleles that modulate a subject's susceptibility to conditions like critical limb ischemia are presently known, there is a need to identify the genetic determinants that play important roles in the treatment and prevention of ischemic injury.

### CROSS REFERENCE TO RELATED APPLICATIONS

This application claims priority to U.S. Provisional Application Ser. Nos. 62/443,930 and 62/444,167, both filed on Jan. 9, 2017, and U.S. Provisional Application Ser. No. 62/448,278, filed on Jan. 19, 2017, all of which are incorporated herein by reference in their entirety for all purposes.

### GOVERNMENT SUPPORT

This invention was made in part with United States government support under the terms of the following grants awarded by the National Institutes of Health (NIH): R00HL103797, R01HL125695, R01AR066660, R21HL118661, R56HL124444, R01HL124444, and F32HL129632. The United States government has certain rights in this invention.

### FIELD

Embodiments of the present disclosure relate generally to the treatment and prevention of ischemic injury. More particularly, embodiments of the present disclosure include materials and methods for treating and preventing ischemic injury caused by diseases like peripheral artery disease (PAD) by modulating various candidate genes within the Lsq-1 QTL, such as Bcl-2-associated athanogene-3 (Bag3).

### BACKGROUND

Critical limb ischemia (CLI) is a manifestation of peripheral artery disease (PAD) that carries significant mortality and morbidity risk in humans. Although progress has been made in elucidating the contribution of various genetic factors to the development of PAD, no alleles that modulate a subject's susceptibility to CLI are presently known. Genetic analysis in inbred mouse strains identified a 37-gene quantitative trait locus (QTL) on chromosome 7 termed Lsq-1, which was associated with tissue survival and perfusion recovery following hind limb ischemia (HLI) induced by femoral artery ligation. To date, only two genes within this QTL (ADAM12 and IL-21R) have been generally associated with the differential perfusion recovery observed among C57BL/6 (BL6), BALB/c, and other inbred strains of mice following HLI.

A notable feature of Lsq-1 is its association not only with perfusion recovery but also with muscle necrosis, raising the possibility that genes related to myogenesis and function might be relevant to ischemic tissue survival. In some instances, muscle function can be an accurate predictor of morbidity/mortality outcomes in PAD, thus the ability of muscle to regenerate and generate force after ischemic injury could be a critical determinant of clinical outcomes. Therefore, there is a need to identify more specifically the genetic determinants that play an important role in the treatment and prevention of ischemic injury caused by various disease conditions like PAD.

### SUMMARY

Embodiments of the present disclosure include a method of treating ischemic injury in a subject, the method comprising administering a polynucleotide encoding a Bcl2-associated athanogene-3 (BAG3) polypeptide to the subject, wherein the BAG3 polypeptide encoded by the polynucleotide is represented by SEQ ID NO:2, and treating at least one symptom associated with the ischemic injury in the subject.

The method according to paragraph [0006], wherein the BAG3 polypeptide encoded by the polynucleotide comprises an isoleucine at amino acid position 79.

The method according to paragraph [0007], wherein the BAG3 polypeptide comprises at least one amino acid substitution in addition to the isoleucine at amino acid position 79.

The method according to any of paragraphs [0006] to [0008], wherein the polynucleotide encoding the BAG3 polypeptide is operably coupled to a targeting vector capable of causing the expression of the BAG3 polypeptide in at least one of a muscle cell, fibroblast, stem cell, pericyte, and endothelial cell.

The method according to any of paragraphs [0006] to [0009], wherein the targeting vector comprises an adeno-associated virus (AAV).

The method according to any of paragraphs [0006] to [0010], wherein the subject has at least one of the following single nucleotide polymorphisms: (i) a leucine at amino acid position 209; (ii) an alanine at amino acid position 63; (iii) a serine at amino acid position 380; and wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury in the subject.

The method according to any of paragraphs [0006] to [0010], wherein the ischemic injury comprises myopathy or vascular deficiency.

The method according to any of paragraphs [0006] to [0011], wherein the ischemic injury is caused by one or more of peripheral artery disease comprising intermittent claudication or critical limb ischemia, muscular dystrophy, myofibrillar myopathy, degenerative myopathies, glycogen storage diseases, trauma, renal disease, atrial fibrillation, COPD, coronary artery disease, morbid obesity, cachexia, congestive heart failure, myocardial infarction, and diabetes mellitus.

The method according to any of paragraphs [0006] to [0012], wherein at least one symptom is necrosis, and wherein administering the polynucleotide encoding the BAG3 polypeptide represented by SEQ ID NO:2 and/or the polynucleotide encoding the BAG3 polypeptide comprising an isoleucine at amino acid position 79 treats the ischemic injury in the subject by reducing necrosis.

The method according to any of paragraphs [0006] to [0012], wherein at least one symptom is myopathy, and wherein administering the polynucleotide encoding the BAG3 polypeptide represented by SEQ ID NO:2 and/or the polynucleotide encoding the BAG3 polypeptide comprising an isoleucine at amino acid position 79 treats the ischemic injury in the subject by reducing the myopathy.

The method according to any of paragraphs [0006] to [0014], wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury by increasing one or more of muscle fiber cross-sectional area, capillary density, muscle function, muscle regeneration, stem cell activity, vascular density, and vascular luminal diameter.

The method according to any of paragraphs [0006] to [0015], wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury by causing one or more of increase in myotube diameter, myotube phenotype, contractile function, an increase in stem cell or satellite cell activity/myogenesis, an increase in mitochondrial number or respiratory function, an increase in autophagic flux, and decreased DNA fragmentation.

The method according to any of paragraphs [0006] to [0016], wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury by causing one or more of increased expression of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker) and muscle RING-finger protein 1 (MuRF-1), and decreased in expression of myostatin.

The method according to any of paragraphs [0006] to [0017], wherein administering the polynucleotide encoding the BAG3 polypeptide represented by SEQ ID NO:2 and/or the polynucleotide encoding the BAG3 polypeptide comprising an isoleucine at amino acid position 79 to the subject comprises one or more of intramuscular injection, percutaneous injection, intraperitoneal injection, intravenous injection, and oral consumption.

The method according to any of paragraphs [0006] to [0018], wherein administering the polynucleotide encoding the BAG3 polypeptide represented by SEQ ID NO:2 and/or the polynucleotide encoding the BAG3 polypeptide comprising an isoleucine at amino acid position 79 to the subject comprises two or more separate injections.

Embodiments of the present disclosure also include a method of preventing ischemic injury in a subject, the method comprising administering a polynucleotide encoding a Bcl2-associated athanogene-3 (BAG3) polypeptide to the subject, wherein the BAG3 polypeptide encoded by the polynucleotide is represented by SEQ ID NO:2; and preventing the onset of at least one symptom associated with the ischemic injury in the subject.

The method according to paragraph [0020], wherein the BAG3 polypeptide encoded by the polynucleotide comprises an isoleucine at amino acid position 79.

The method according to paragraph [0021], wherein the BAG3 polypeptide comprises at least one amino acid substitution in addition to the isoleucine at amino acid position 79.

The method according to any of paragraphs [0020] to [0022], wherein the polynucleotide encoding the BAG3 polypeptide is operably coupled to a targeting vector capable of causing the expression of the BAG3 polypeptide in at least one of a muscle cell, fibroblast, stem cell, pericyte, and endothelial cell.

The method according to any of paragraphs [0020] to [0023], wherein the subject has at least one of the following single nucleotide polymorphisms: (i) a leucine at amino acid position 209; (ii) an alanine at amino acid position 63; (iii) a serine at amino acid position 380; and wherein administering the polynucleotide encoding the BAG3 polypeptide represented by SEQ ID NO:2 and/or the polynucleotide encoding the BAG3 polypeptide comprising an isoleucine at amino acid position 79 prevents the onset of the at least one symptom associated with the ischemic injury in the subject.

Embodiments of the present disclosure also include a pharmaceutical composition for treating or preventing ischemic injury in a subject, the composition comprising a polynucleotide encoding a Bcl2-associated athanogene-3 (BAG3) polypeptide, wherein the BAG3 polypeptide encoded by the polynucleotide is represented by SEQ ID NO:2; and a pharmaceutically acceptable excipient.

The composition according to paragraph [0025], wherein the BAG3 polypeptide encoded by the polynucleotide comprises an isoleucine at amino acid position 79.

The composition according to paragraph [0026], wherein the BAG3 polypeptide comprises at least one amino acid substitution in addition to the isoleucine at amino acid position 79.

The composition according to any of paragraphs [0025] to [0027], wherein the polynucleotide encoding the BAG3 polypeptide is operably coupled to a targeting vector capable of causing the expression of the BAG3 polypeptide in at least one of a muscle cell, fibroblast, stem cell, pericyte, and endothelial cell.

The composition according to any of paragraphs [0025] to [0028], wherein the composition is formulated for administration to a subject by one or more of intramuscular injection, percutaneous injection, intraperitoneal injection, ingestion, and intravenous injection.

Embodiments of the present disclosure also include a kit comprising a pharmaceutical composition comprising a polynucleotide encoding a Bcl2-associated athanogene-3 (BAG3) polypeptide, wherein the BAG3 polypeptide encoded by the polynucleotide comprises an isoleucine at amino acid position 81, and a pharmaceutically acceptable excipient, and a delivery device for administering the pharmaceutical composition to a subject.

The kit according to paragraph [0030], wherein the BAG3 polypeptide encoded by the polynucleotide comprises an isoleucine at amino acid position 79.

The kit according to paragraph [0031], wherein the BAG3 polypeptide comprises at least one amino acid substitution in addition to the isoleucine at amino acid position 79.

### BRIEF DESCRIPTION OF THE DRAWINGS

FIGS. 1A-1F. Identification of Bag3 as a target for HLI-induced tissue necrosis. (A) BL6, BALB/c, and Congenic mice (C.B6-Lsq1-3, also known as C.B6-Civq1-3; N≥5 mice per strain) were subjected to HLI and limb necrosis was assessed using a semi-quantitative scoring system. *P=NS vs. BL6. †P<0.05 vs. BALB/c. (B) Representative images of Congenic contralateral control (R) and ischemic limb (L) muscle morphology (H&E), regeneration (dystrophin, eMyHC), and vascular morphology (CD31, SMA). Dyst, dystrophin (N=4 mice/strain, scale bar=100 μm). (C-E) Quantification of strain dependent non-myofiber area (C), eMyHc⁺ myofibers (D), and CD31⁺ capillary density (E) on d7 after HLI (N=4 mice/strain). *P<0.05 vs. BL6 and Congenic. (F) Gastrocnemius BAG3 mRNA expression (corrected for GAPDH and normalized to BL6 control) after HLI in BL6 (black bars) and BALB/c (gray bars) mice (N=4 mice/strain/day). *P<0.05 vs. strain-specific Control. †P<0.05 vs. strain-specific HLI d1. Necrosis data plotted per mouse, with means±SEM. All other data are means±SEM.

FIGS. 2A-2H. Strain-specific coding variants of BAG3 differentially promote limb muscle hypertrophy and capillary density in non-ischemic muscle. A-B. Intramuscular injection into BALB/c mice of adeno-associated viruses encoding BAG3^{Met81} or BAG3^{Ile81} (N=4 mice/virus) resulted in similar upregulation of Bag3 mRNA on day 3 post-injection (A) and FLAG-tagged BAG3 protein on day 7 post-injection (B) compared to mice injected with AAV-GFP. *P<0.05 vs. GFP. IP, immunoprecipitation; WB, western blot. (C) Representative dystrophin staining (pseudocolored green; scale bar=100 μm) of TA muscle 14 days post-injection. (D) Quantification of myofiber CSA on days 7 and 14 post-injection (N=4 mice/virus). ‡P<0.05 vs. GFP or BAG3^{Met81}. (E) Representative images of CD31 and smooth muscle actin (SMA) staining 7 days post-injection (scale bar=100 μm). (F) Quantification of CD31⁺ vessel density in TA muscle. *, P<0.05 vs. GFP (N=4 mice/virus). (G-H) Limb perfusion was analyzed by LDPI on day 7 post-injection (N=10 mice/virus) (G) and quantified (H) as a percentage of perfusion in the non-injected limb.

FIGS. 3A-3F. BAG3^{Ile81} expression regulates ischemic limb tissue necrosis and perfusion. BL6 (N=5) and BALB/c mice were injected IM with AAVs (N=29 GFP; N=19 BAG3^{Met81}; N=19 BAG3^{Ile81}) and 7 days later subjected to HLI. (A) Semi-quantitative scoring of limb muscle necrosis. *P<0.05 vs. GFP. †P<0.05 vs. BAG3^{Met81}. (B) Representative MR T2-weighted and MR angiography images and (C) quantification of MR ADC perfusion at HLI d7 (N=4 BL6; N=8 GFP; N=7 BAG3^{Met81}; N=6 BAG3^{Ile81}). *P<0.05 vs. Control; **P<0.05 vs. BL6; †P<0.05 vs. GFP; ‡P<0.05 vs. BAG3^{Met81}. (D-E) Representative images of CD31 and SMA staining (scale bar=100 μm) (D) for quantification of CD31⁺ vessel density (E) at HLI d7 (N≥4 mice/group). *P<0.05 vs. Control; P<0.05 vs. GFP or BAG3^{Met81}. (F) Virus-injected BALB/c mice (N=16 mice/virus) were subjected to HLI for 21d, and limb necrosis score distribution was determined. *P<0.05 vs. GFP; †P<0.05 vs. BAG3^{Met81}. Necrosis data plotted per mouse, with means±SEM. All other data are means±SEM.

FIGS. 4A-4K. BAG3^{Ile81} rescues ischemic BALB/c muscle morphology and regeneration. BL6 (black bars) and BALB/c (gray bars) mice injected IM with AAVs were subjected to HLI for 7 days. The TA was analyzed histologically with H&E (A; scale bar=100 μm), and non-myofiber area (B) and myofiber cross sectional area (C) were quantified (N≥5 mice/group). Note the lack of fascicular myofiber arrangement and absence of centralized myofiber nuclei in GFP and BAG3_{Met81} groups, which were rescued by BAG3^{Met81}. (D) Representative immunofluorescence images of TA muscle labeled with antibodies against embryonic heavy chain (eMyHC) and dystrophin (pseudocolored green; scale bar=100 μm) were used to quantify eMyHc⁺ myofiber number (E) and size (F) (N2≥5 mice/group). There were very few eMyHc⁺ fibers in contralateral non-ischemic control limbs. (G-H) Gastrocnemius mRNA expression of myogenin (G) and Tmem8c (myomaker) (H) were determined by qRT-PCR (N≥5 mice/group, corrected for GAPDH and normalized to contralateral control). (I) Representative images of primary muscle progenitor cells from BALB/c mice that were infected with AAVs encoding GFP, BAG3^{Met81}, or BAG3^{Ile81} then differentiated into myotubes and labeled with DAPI and anti-MyHC (N≥3 group). (J) Quantification of myoblast fusion index. (K) Quantification of fusion index for C3H-10T1/2 pluripotent pericyte cells and C2C12 myoblasts mixed at a 75:25 ratio and infected with the indicated AAVs (N≥3 group). *P<0.05 vs. Control; †P<0.05 vs. BL6; ‡P<0.05 vs. GFP or BAG3^{Met81}. All data are means±SEM.

FIGS. 5A-5F. BAG3^{Met81} is not a dominant negative inhibitor in ischemic BL6 muscle. BL6 mice were infected with AAV6 encoding BAG^{Met81} for 7 days and subjected to 7 days of HLI (N≥5 mice/group). (A) Representative H&E-stained and IF images labeled for dystrophin, PAX7, eMyHC, CD31, and SMA (scale bar=100 μm) were used to quantify non-myofiber tissue area (B), PAX7⁺ nuclei (C), eMyHC expression (D), size of eMyHc⁺ myofibers (E), and capillary density (F). All data are means±SEM.

FIGS. 6A-6I. Systemic BAG3^{Ile81} delivery rescues BALB/c limb muscle blood flow and function after ischemia. BL6 (black bars, N=5) and BALB/c (gray bars) mice injected IV with AAVs encoding GFP (N=5), BAG3^{Met81} (N=10), or BAG3^{Ile81} (N=9) were subjected to modified HLI with collateral vessels left intact. (A) Quantification of skeletal muscle BAG3 mRNA expression by qRT-PCR, corrected for GAPDH and normalized to contralateral control. (B) Limb necrosis score distribution. (C) Representative LDPI images of paw blood flow. (D) Quantification of paw perfusion by LDPI. (E-F) Force-frequency analysis of isolated extensor digitorum longus (EDL) muscles from the contralateral control (E) and ischemic (F) limbs at HLI d7. (G) Peak specific EDL muscle force (expressed as a % of the contralateral EDL). (H) Correlational analysis between BAG3 protein expression and muscle peak specific force. (I) Representative H&E stains for TA muscle morphological analysis (scale bar=100 μm). †P<0.05 vs. BL6; ‡P<0.05 vs. GFP or BAG3^{Met81}. Necrosis data plotted per mouse, with means±SEM. All other data are means±SEM.

FIGS. 7A-7F. Autophagy is differentially regulated in ischemic BALB/c and BL6 muscle and by BAG3 variants. (A) BL6 and BALB/c mice were subjected to HLI for 3 or 7-days (N≥5 mice per strain, per time-point) and gastrocnemius LC3 mRNA expression (corrected for GAPDH and normalized to BL6 control) was determined by qRT-PCR. *P<0.05 vs. strain-specific control. †P<0.05 vs. strain-specific HLI d3. (B-C) LC311 protein abundance was determined in HLI d7 gastrocnemius (Gastroc) by western blotting using GAPDH as a loading control (B) and quantified relative to GAPDH and normalized to non-ischemic BL6 controls (C). *P<0.05 vs. strain-specific control. (D) BALB/c mice injected IV with AAVs encoding GFP (N=5), BAG3^{Met81} (N=6), or BAG3^{Ile81} (N=6) were subjected to modified HLI with collateral vessels left intact, and gastroc LC3 mRNA expression (corrected for GAPDH and normalized to BL6 control) was determined by qRT-PCR. *P<0.05 vs. control. †P<0.05 vs. GFP or BAG3^{Met81}. (E-F) LC3bII protein abundance was determined in Gastroc muscle lysates from BALB/c mice injected IV with AAVs by western blotting using GAPDH as a loading control (E) and quantified relative to GA PDH and normalized BL6 controls (F). In FIG. 7E, all bands are from the same blot and exposure, and the vertical line indicates where the blots were cropped and lanes spliced together for comparison. †P<0.05 vs. GFP or BAG3^{Met81}. All data are means±SEM.

FIGS. 8A-8D. BAG3^{Ile81} differentially binds HspB8 and rescues ischemic autophagic flux in BALB/c muscle cells. (A) BALB/c primary muscle cells were infected with viruses encoding GFP, BAG3^{Met81}), or BAG3^{Ile81} and allowed to differentiate for 120 h before experimental ischemia (3HND). Whole cell lysates were immunoblotted for HspB8 and SQSTM1 (p62). (B) FLAG-BAG3 was immunoprecipitated from total cell lysates (A) to examine the expression of BAG3 protein and the association of HspB8 and SQSTM1 with exogenously expressed BAG3. All bands in each blot are from the same membrane and exposure time, and the vertical lines indicate where the blots were cropped and lanes spliced together for comparison. (C-D) To examine autophagic flux, BALB/c primary myoblasts (C) or myotubes differentiated for 120 h (D) were infected with an adenovirus expressing membrane-localized red fluorescent protein (mRFP-EGFP-LC3) and adeno-associated viruses (AAV6) encoding a luciferase control, BAG3^{Met81}, or BAG3^{Ile81} then subjected to experimental ischemia (3HND). Bafilomycin A1 (200 nM) was used as a positive control. Representative IF images of mRFP-EGFP-LC3 in BALB/c myoblasts (C) and myotubes (D) are shown (left panels), and the percentage of autolysosomes (mRFP+/eGFP-puncta) were quantified (right panels). *P<0.05 vs. BL6.

FIG. 9. Congenic mice narrowing Lsq-1. BALB/c-Chr7-C57BL/6J chromosome substitution congenic strain (Congenic, C.B6-Lsq1-3) was generated in which a 12 MB region of Chr 7 (containing Bag3, among other Lsq-1 genes), was introgressed from BL6 into the BALB/c background.

FIG. 10. BAG3 protein variation around amino acid residue 81. Alignment of BAG3 protein sequences from various species reveals a lack of conservation at amino acid residue 81 but a high degree of conservation among surrounding residues.

FIGS. 11A-11B. Localization of AAV6-expressed FLAG-tagged BAG3. To verify the efficiency of expression of AAV6-BAG3, 2×10¹⁰ active viral particles (AVP) were injected into the TA muscle of non-ischemic mice. (A-B) Muscle sections (8 μm) were immunofluorescently stained with anti-FLAG (red) and anti-CD31 (green) and co-labeled with antibodies against smooth muscle actin (SMA, white, A) or dystrophin (blue, B), and co-labeled with DAPI to stain nuclei (A) to verify efficiency of muscle tissue transgene expression.

FIGS. 12A-12B. Verification of BAG3 expression in ischemic limb muscle. BL6 and BALB/c mice were infected with serotype 6 adeno-associated viruses encoding GFP, BAG3^{Met81}, or BAG3^{Ile81}. Seven days later, mice were subjected to HLI, and another 7 days later tissue was harvested for analysis of BAG3 mRNA and protein expression. (A) BALB/c skeletal muscle homogenates were western blotted for BAG3 protein expression and α-tubulin as a loading control, and non-ischemic muscle was used as a Control. (B) Musde from control, non-ischemic hind limb or from ischemic BL6 mice (black bar) or ischemic BALB/c mice injected with AAVs encoding the indicated proteins (gray bars) was used for qRT-PCR analysis. *P<0.05 vs. non-ischemic control (Control); †P<0.05 vs. GFP.

FIGS. 13A-13B. AAV-infected mice display similar perfusion deficits immediately post ischemia surgery. BALB/c were infected with adeno-associated viruses encoding GFP, BAG3^{Met81}, or BAG3^{Ile81} and 7 days later were subjected to HLI. Limb blood flow was analyzed by LDPI immediately post-surgery (A) and quantified as a percentage of perfusion in the non-injected limb (B).

FIGS. 14A-14C. BAG3^{Ile81} enhances non-ischemic muscle regeneration. Cardioloxin (CTX, Naja nigricolis toxin) injection, a traditional muscle regeneration model, was performed in BALB/c mice injected IM with AAVs encoding GFP, BAG3^{Met81}, or BAG3^{le81} (N≥5 mice/group). The TA muscle was visualized histologically after H&E staining (A, scale bar=100 μm), and non-myofiber tissue area (B) and myofiber cross sectional area (C) were quantified. Note the preservation of myofiber fascicular architecture and size with BAG3^{Ile81} expression. *P<0.05 vs. Control. ‡P<0.05 vs. GFP or BAG3^{Met81}. All data are means±SEM.

FIGS. 15A-15E. BAG3 overexpression does not alter myoblast proliferation. (A-B) BL6 and BALB/c mice were injected IM with the indicated AAVs then subjected to HLI for 7-days (N≥5 mice/group). TA muscle sections were stained with antibodies against the myogenic precursor cell marker PAX7 and dystrophin (Dyst, pseudocolored green) (A), and the density of PAX7⁺ nuclei was quantified (B). *P<0.05 vs. Control. C-D. C2C12 myoblasts (C) or primary myoblasts isolated from BALB/c mice (D) were infected in vitro with Adenoviruses encoding GFP, BAG3^{Met81}, or BAG3^{Ile81} and cell numbers were assessed at the indicated times as an indicator of proliferation (N≥3). (E) Viral knockdown of BAG3 (Bag3^{sh}, N≥3) decreases cell number/proliferation in C2C12 cells in vitro. *P<0.05 vs. GFP control. All data are means±SEM.

FIGS. 16A-16B. Strain dependence of autophagy-related transcripts during limb ischemia. (A) BL6 and BALB/c mice were subjected to HLI for 3 and 7 days, and RNA was isolated from limb muscle tissue for the quantification of the autophagy-related mRNAs ULK1, ATG7, Gabarap, SQSTM1, and CTSL by qRT-PCR, corrected for GAPDH, and normalized to expression in the contralateral control limb. *P<0.05 vs. strain-matched Control; †P<0.05 vs. strain-matched HLI d3. #P<0.05 vs. BL6 Control (a priori analysis). (B) BALB/c mice (gray bars) injected IV with AAVs encoding GFP (N=5), BAG3^{Met81} (N=10), or BAG3^{Ile81} (N=9) were subjected to modified HLI with collateral vessels left intact. RNA was isolated from limb muscle tissue for the quantification of autophagy-related mRNAs (ULK1, ATG7, Gabarap, SQSTM1, and CTSL) by qRT-PCR, corrected for GAPDH and normalized to expression in the contralateral control limb. *P<0.05 vs. Control; †P<0.05 vs. HLI d7. All data are means±SEM.

FIG. 17. Differential expression of BAG3 protein interactors during HLI in BL6 and BALB/c limb muscle. BL6 and BALB/c mice were subjected to HLI for 1 and 3 days, and protein was isolated from the soleus and plantaris limb muscles for western blotting. GAPDH was used as a loading control.

FIGS. 18A-18F. Hypoxia and nutrient deprivation induces ubiquitination and loss of BAG3 protein in skeletal myocytes. (A) Immortalized mouse C2C12 myocytes and rat L6 myocytes or primary mouse myocytes were subjected to 3 h hypoxia and nutrient deprivation (HND) and cell lysates were probed for BAG3 expression and tubulin as a loading control. (B) L6 cells were subjected to the indicated stress inducers, and effects on BAG3 protein were analyzed by Western blotting. (C) L6 cells were subjected to 1 h HND and BAG3 was immunoprecipitated (IP) and probed with an antibody against poly-ubiquitin (pUb) and BAG3. (D-E) Effects of 3 h HND on BAG3 recovery for the indicated times in normal O₂ and glucose were tested in C2C12 (D) and L6 cells (E). (F) Effects of 3 h HND on BAG mRNA were evaluated by quantitative real-time (qRT) PCR in C2C12 and L6 at the indicated times of recovery in normal O₂ and glucose. BAG3 mRNA was normalized to that of β-glucuronidase in all samples.

FIGS. 19A-19E. The BAG3^{Ile81} polymorphism rescues defects in the skeletal muscle response to HND. (A) Rat L6 myotubes were transfected with GFP only or GFP plus BAG3^{Ile81} (here designated BAG3^{B6}, as this is the polymorphism in C57BL/6 mice) or BAG3^{Met81} (designated BAG3^{Balb/C}, as this polymorphism is present in BALB/c mice) and subjected to 3 h HND and 24 h recovery and BAG3 expression was analyzed by Western blotting. (B) Representative photomicrographs of L6 myotubes transfected with BAG3 constructs demonstrate efficient transfection based on GFP expression and that BAG3^{Ile81} limits myotube atrophy. (C) L6 myotube diameter was quantified after 3 h HND or an additional 24 h recovery in normal O₂ and nutrients in cells transfected with the indicated constructs. (D-E) L6 myotubes were treated as described in FIGS. 19A-C and changes in mitochondrial content (D) and DNA fragmentation (E) were quantified. In all cases, BAG3^{Ile81} (BAG3^{B6}) significantly improved outcomes, particularly after 24 h recovery.

FIGS. 20A-20D. BAG3^{Ile81} improves expression of angiogenic genes after experimental hypoxia and nutrient deprivation. L6 myotubes were treated as described in FIG. 2, and RNA was harvested for qRT-PCR to analyze expression of (A) vascular endothelial growth factor (VEGF); (B) Neuropilin (Nrp)-1; (C) VEGF receptor (R)-1 (Flt); and (D) VEGFR-2 (Flk). BAG3^{Ile81} (B6) significantly improved expression of these genes compared to BAG3^{Ile81} (Balb/c) at various time points of HND or recovery.

FIGS. 21A-21D. BAG3^{Ile81} improves expression of genes involved in myocyte survival and differentiation after experimental hypoxia and nutrient deprivation. L6 myotubes were treated and analyzed as described in FIG. 3, and expression of (A) HIF-1α; (B) myogenin; (C) MAFbx; and (D) MuRF-1 was analyzed at the indicated time points. BAG3^{Ile81} (B6) significantly improved expression of these genes compared to BAG3^{Met81} (Balb/c) at various time points of HND or recovery.

FIGS. 22A-22F. Generation of an adeno-associated virus (AAV) encoding BAG3^{Ile81}. (A) Differentiated myotubes were co-infected with helper adenovirus (AdGFP) and AAV-Luciferase (Luc) at low and high titers, and expression was analyzed by bioluminescence imaging after addition of luciferin, demonstrating that muscle cells can be infected well with AAV. (B) Western blots for luciferase from the experiment in (A) show virus dose-dependent expression of the Luc transgene. (C) Mice were infected intramuscularly with two different capsid isotypes of AAV, AAV9 and a modified AAV3b-based vector termed SASTG, both expressing Luc. Mice were injected intraperitoneally with luciferin and underwent bioluminescence imaging at the indicated times. (D) Dot blot analysis of AAV-BAG3 titer. (E) Representative fluorescence micrographs of 911 epithelial cells, ECRF endothelial cells, primary mouse myoblasts, and differentiated primary mouse myotubes infected with helper adenovirus (AdGFP) and AAV-BAG3^{Ile81}. (F) Cells in FIG. 22E and uninfected control (Con) cells were lysed and Western blotted with antibodies against BAG3 and tubulin.

FIGS. 23A-23F. Intramuscular delivery of AAV-BAG3^{Ile81} rescues limb necrosis after bind limb ischemia (HLI) in BALB/c mice. (A) Representative photographs of hind limbs 7 days after surgical HLI demonstrates perfusion recovery in C57BL/6 (BL6) mice and marked tissue necrosis in BALB/c mice. (B) Representative photographs of hind limbs 7 days after surgical HLI in BALB/c mice treated with i.m. AAV-BAG3^{Ile81} demonstrates rescue of perfusion recovery. (C) Semi-quantitative necrosis score in BL6 and BALB/c mice at the indicated time points after surgical HLI. AAV-BAG3^{Ile81} (BAG3^{BL6}) completely prevented the development of limb necrosis in BALB/c mice. NR, none recorded. (D) Ischemic gastrocnemius muscles were excised at the indicated times after HLI and weighed. (E) Muscle tissue homogenates were isolated 7 d after HLI (14 d post-injection, left) or 7 d post-injection (right) and analyzed by Western blotting with the indicated antibodies. (F) Muscle RNA was harvested from non-ischemic right limbs (Sham) or ischemic left limbs 7 d after HLI in mice treated without or with AAV-BAG3^{Ile81} (BAG3^{BL6}) and used to analyze BAG3 mRNA expression by qRT-PCR. AAV-BAG3^{Ile81} rescued mRNA expression without overexpression.

FIGS. 24A-24E. Intramuscular delivery of AAV-BAG3^{Ile81} rescues muscle atrophy and improves angiogenesis and vessel size after hind limb ischemia (HLI) in BALB/c mice. Delivery of AAV-BAG^{Ile81} improved measures of (A) muscle fiber cross-sectional area (CSA), (B) capillary density, (C) lumen+blood vessels (i.e., large vessels), and (D) number of larger myofibers. (E) Representative immunofluorescence micrographs of skeletal muscle tissue sections from uninjected contralateral non-ischemic limb (top) or AAV-BAG3^{Ile81}-injected ischemic limb (bottom) from BALB/c mice 7 d after HLI. Sections were immunostained with anti-dystrophin (blue) to outline myofibers and anti-CD31 (green) to identify endothelial cells.

FIGS. 25A-25E. Intramuscular injection of AAV-BAG3^{Ile81} rescues ischemia-induced abnormalities in muscle gene expression after hind limb ischemia (HLI) in BALB/c mice. Total RNA was harvested from muscle tissue of non-ischemic right limbs (Sham) or ischemic left limbs 7 d after HLI in mice treated without or with AAV-BAG3^{Ile81} (BAG3^{BL6}) and qRT-PCR was used to analyze mRNA expression 1 of (A) cyclin D-1, (B) Pax7, (C) MyoD, (D) myostatin, and (E) myogenin. AAV-BAG3^{Ile81} significantly altered expression of most of these genes compared to untreated BALB/c mice and in some cases compared to BL/6 mice.

FIGS. 26A-26E. Intramuscular injection of AAV-BAG3^{Ile81} has significant effects on muscle fiber size and vascularity in non-ischemic muscle. AAV-BAG3^{Ile81} was injected into non-ischemic left tibialis anterior (LTA) muscle of BALB/c mice, and muscle tissue was harvested 7 d later. Compared to contralateral right TA muscle of treated mice (Sham RTA) or muscle from uninjected mice (Cont. RTA), treatment improved measures of (A) muscle fiber cross-sectional area (CSA), (B) capillary density, (C) lumen+blood vessels (i.e., large vessels), and (D) number of larger myofibers, even in the absence of ischemia. (E) Representative immunofluorescence micrographs of skeletal muscle tissue sections from uninjected contra lateral right TA muscle (top) or AAV-BAG3^{Ile81}-injected left TA muscle (bottom) of BALB/c mice 7 d after injection. Sections were immunostained with anti-dystrophin (blue) to outline myofibers and anti-CD31 (green) to identify endothelial cells.

FIGS. 27A-27C. Alterations in BAG3 expression or mutations in BAG3 alter its ability to improve muscle cell function. (A) BALB/c primary myoblasts were infected with recombinant adenovirus expressing full-length human (FL-Hu) BAG3 or BAG3 with a known functional mutation that contributes to hereditary myofibrillar and cardiomyocyte myopathy (P209L) and examined for the ability to differentiate (form myotubes, indicated by myosin heavy chain (MyHC)-positive myofibers with multiple nuclei, in red. (B-C) The fusion of myoblasts into myotubes (differentiation; Fusion Index %) and the size of the myotubes (maturation; % Area MyHC⁺) were both improved by expression of full-length human BAG3 but not P209L.

FIGS. 28A-28F. Cell-specific effects of BAG3 in primary liver and skeletal muscle endothelial cells (EC) from BL6 and BALB/c mice. (A) BAG3 protein expression is shown, which was stable in abundance in these cells even after 3 h of hypoxia and nutrient deprivation (HND) in vitro compared to control (Con) normoxic cells. Similarly, BAG3 protein abundance was relatively unchanged in immortalized ECRF ECs and in HUVECs after short (3 h) or intermediate duration (8 h) ischemia, but was reduced after 24 h ischemia in these cells in vitro (B). Knock down of BAG3 in HUVECs with an adenovirus encoding a BAG3 shRNA or a control, scrambled (Scr) shRNA (C); cellular apoptosis (Annexin V staining) and necrosis (propidium iodide staining) after 24 h ischemia was examined (D). Cellular apoptosis and necrosis were increased in control-treated cells after 24 h ischemia and were exacerbated by BAG3 knockdown (E-F).

BAG3 knockdown (shRNA) or overexpression of full-length human (FL-Hu) BAG3 or BAG3 with a known functional mutation that contributes to hereditary myofibrillar and cardiomyocyte myopathy. Primary human umbilical vein endothelial cells (HUVECs) were examined for the ability to form mesh/loop networks (angiogenesis assay) after 8 h of normoxia or hypoxia and nutrient deprivation (HND). (A) Loop formation was visualized by immunofluorescence imaging of phalloidin. Quantification of the number of loops demonstrated that the loss of BAG3 expression prevented angiogenesis in either normoxic or hypoxic conditions, while expression of either full-length human BAG3 or the P209L mutant improved loop formation under both conditions (B-C).

FIGS. 30A-30C. Endothelial cell autophagic flux in human primary endothelial cells (HUVECs). HUVECs were co-infected with an RFP-GFP-LC3 reporter adenovirus and either an empty control adenovirus or viruses expressing BAG3shRNA, full-length human (FL-Hu) BAG3, or the human P209L mutant. (A) Autolysosome formation was visualized by immunofluorescence imaging. The loss of BAG3 expression reduced autolysosome formation after both short- (3 h) and long-term (24 h) HND (B-C). Full-length human BAG3 or P209L did not alter autolysosome formation after 3 h ischemia, but the two variants had differential effects on autolysosome formation after prolonged (24 h) ischemia, indicating a lack of effectiveness of P209L to improve EC autophagic flux during longer periods of ischemia.

FIGS. 31A-31B. Effects of BAG3 knockdown or mutation on endothelial cell proliferation. Human primary endothelial cells (HUVECs) were infected with viruses expressing BAG3shRNA, full-length human BAG3, or the human P209L mutant. (A) Proliferation over 72-hours was visualized by imaging cell number after exposure to nuclear dye. (B) The loss of BAG3 expression suppressed HUVEC numbers after 48- and 72-hours of culture. Full-length human BAG3 overexpression improved HUVEC proliferation after 48- and 72-hours of culture. There was no effect of BAG3-P209L on HUVEC proliferation, indicating the ability of BAG3 to increase HUVEC proliferation in the absence of a functional mutation.

FIGS. 32A-32E. Analysis of the roles of specific BAG3 domains (A). (B-C) Overexpression of full-length (FL) BAG3 improved primary myoblast differentiation/fusion as shown by increased myonuclei within myosin heavy chain (MyHC)-positive myofibers. Consistent with our prior results in which a SNP adjacent to the first IPV domain (Ile81Met) disrupted differentiation, the benefits conferred by BAG3-FL were lost when either the IPV1 or IPV2 domain was deleted. (D-E) Using tandem-fluorescence LC3 reporter (tfLC3), a role for the IPV1 and IPV2 domains in myofiber autophagic flux during ischemia is shown.

FIGS. 33A-33B. Validation of the effectiveness of human BAG3 to rescue a dysfunctional murine autophagy phenotype in muscle cells. Autophagic flux is rescued in BALB/c muscle myotubes by overexpression of full-length human (FL-Hu) BAG3. (A-B) Using the tandem-fluorescence LC3 reporter, similar effectiveness of human BAG3 to improve BALB/c myofiber autophagic flux during ischemia is shown.

FIGS. 34A-34B. Clinical relevance of BAG3 in human peripheral artery disease. (A) Limb gastrocnemius muscle tissues were collected from healthy human adults (age-matched, HA), patients with intermittent claudication (IC), and patients with critical limb ischemia (CLI) and protein expression of BAG3, HspB8, LC3I and II, and GAPDH was examined by western blotting. (B) Densitometry demonstrated a slight increase in BAG3, HspB8, and LC3B proteins in limb tissues from CLI patients.

FIGS. 35A-35B. Cellular specificity of BAG3 protein expression in muscle cells isolated from gastrocnemius muscle tissues from subjects described in FIG. 34. (A) Primary MPCs were differentiated into myotubes, and protein expression was analyzed by western blotting. (B) Densitometry demonstrated a reduction in BAG3 and HspB8 proteins in myotubes from CLI patients.

FIGS. 36A-36B. The ability of MPCs from CLI patients to differentiate. (A) MPC differentiation (myotube formation), as indicated by myosin heavy chain-positive myofibers, in red, with multiple nuclei. (B-C) The fusion of myoblasts into myotubes (differentiation; Fusion Index %) and the size of the myotubes (maturation; % Area MyHC) were both attenuated in CLI patient myotubes.

FIGS. 37A-37F. SNPs found to be expressed preferentially in heart failure patients of African-American (AA) descent (hereafter designated BAG3^{AA-SNP}) have dominant inhibitory effects in cardiomyocytes. AAVs encoding several of these human (hu) variants were generated, including P63A and P380S, as well as a P63A/P680S double mutant virus. BALB/c MPCs were infected with these viruses and effects on myoblast fusion, myotube maturation, and autophagy were evaluated. (A-C) Staining for myosin heavy chain (MyHC) and DAPI demonstrated that BAG3^{AA-SNP} decreased myoblast fusion and myotube maturation, and co-expression of a tflLC3 reporter demonstrated that BAG3^{AA-SNP} inhibited autophagic flux, as indicated by the presence of green and/or yellow puncta after 3 h of experimental ischemia (D). AAV-mediated overexpression of huWTBAG3, but not huP63A, huP380S, or huP63A/P380S rescued BALB/c muscle morphological abnormalities observed histologically (E) as well as mitochondrial function measured in permeabilized myofibers after 24 h HLI (F).

FIG. 38. Known human variants in BAG3 alter muscle cell differentiation, fusion, and autophagy. AAVs used to express the BAG3AA-SNP in BALB/c mouse myotubes demonstrated that the P63A/P380S double mutant inhibited basal myotube mitochondrial respiration after 3 h HND.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “administering” refers to an administration that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. Administering can be performed using transdermal microneedle-array patches. The term “parenteral” includes subcutaneous, intravenous, intramuscular, intra-articular, intra-synovial, intrasternal, intrathecal, intrahepatic, intralesional, and intracranial injections or infusion techniques. “Biocompatible” generally refers to a material and any metabolites or degradation products thereof that are generally non-toxic to the recipient and do not cause any significant adverse effects to the subject.

The terms “carrier” or “pharmaceutically acceptable carrier” means a carrier or excipient that is useful in preparing a pharmaceutical or therapeutic composition that is generally safe and non-toxic, and includes a carrier that is acceptable for veterinary and/or human pharmaceutical or therapeutic use. As used herein, the terms “carrier” or “pharmaceutically acceptable carrier” encompasses can include phosphate buffered saline solution, water, emulsions (such as an oil/water or water/oil emulsion) and/or various types of wetting agents. As used herein, the term “carrier” encompasses any excipient, diluent, filler, salt, buffer, stabilizer, solubilizer, lipid, stabilizer, or other material well known in the art for use in pharmaceutical formulations and as described further below.

The terms “identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

The term “sequence identity” generally refers to the extent to which two optimally aligned DNA or peptide sequences are identical. An optimal sequence alignment is created by manually aligning two sequences (e.g., a reference sequence, such as a wildtype sequence, and another DNA/peptide sequence) to maximize the number of nucleotide matches in the sequence alignment with appropriate internal nucleotide insertions, deletions, or gaps. As used herein, the terms “percent sequence identity” or “percent identity” or “% identity” is the identity fraction multiplied by 100. The “identity fraction” for a DNA/peptide sequence optimally aligned with a reference sequence is the number of nucleotide/amino acid matches in the optimal alignment, divided by the total number of nucleotides/amino acids in the reference sequence (e.g., the total number of nucleotides/amino acids in the full length of the entire reference sequence.

Thus, one embodiment of the present disclosure provides a Bag3 DNA molecule or BAG3 peptide comprising a sequence that, when optimally aligned to a reference sequence, provided herein as SEQ ID NOs:1 or 2, has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to a reference sequence (e.g., wildtype Bag3 nucleotide sequence or wildtype BAG3 polypeptide sequence).

The term “isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

As used herein, the term “pharmaceutically acceptable” can refer to a component that is not biologically or otherwise undesirable. For example, the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The terms “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure [e.g., an oxysterol] are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. For example, a therapeutically effective amount of a compound of a disclosed oxysterol may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The terms “treat,” “treated,” or “treating,” as used herein, refer to a therapeutic method wherein the object is to slow down (lessen or reduce) an undesired physiological condition, disorder or disease, or to obtain beneficial or desired clinical results. In some aspects of the present disclosure, beneficial or desired clinical results include, but are not limited to, alleviation and/or reduction of symptoms (e.g., myopathy); diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

The term “variant” with respect to a peptide or polypeptide (e.g., BAG3) that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

The term “vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome (e.g., AVV). Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Before any embodiments of the present disclosure are explained in detail, it is to be understood that the present disclosure is not limited in its application to the details of construction and the arrangement of components set forth in the following description or illustrated in the accompanying drawings. The present disclosure is capable of other embodiments and of being practiced or of being carried out in various ways.

Other aspects of the invention will become apparent by consideration of the detailed description and accompanying drawings.

### DETAILED DESCRIPTION

Embodiments of the present disclosure relate generally to the treatment and prevention of ischemic injury. More particularly, embodiments of the present disclosure include materials and methods for treating and preventing ischemic injury caused by diseases like peripheral artery disease (PAD) by modulating various candidate genes within the Lsq-1 QTL, such as Bcl-2-associated athanogene-3 (Bag3). Given that no alleles that modulate a subject's susceptibility to conditions like critical limb ischemia are presently known, there is a need to identify the genetic determinants that play important roles in the treatment and prevention of ischemic injury.

In some instances, muscle function can be an accurate predictor of morbidity/mortality outcomes in PAD, thus the ability of muscle to regenerate and generate force after ischemic injury could be a critical determinant of clinical outcomes. Therefore, the effects of genetic variants in various candidate genes within Lsq-1 were investigated, including the candidate gene Bcl-2-associated athanogene-3 (Bag3). Bag3 has been shown to be involved in skeletal muscle cell biology. BAG3 is involved in myofibrillar integrity through its interactions with HSP70 and CAPZ, and variants in BAG3 have been generally associated with myofibrillar myopathy and dilated cardiomyopathy in humans. In some instances, loss of BAG3 in mice causes perinatal lethality due to fulminant skeletal myopathy.

Embodiments of the present disclosure relate to the modulation of BAG3 to treat and prevent various ischemic conditions. In some embodiments, adeno-associated virus (AAV)-mediated expression of wildtype and BAG3 variants in BL6 and BALB/c mice in a model of limb ischemia, in a toxin model of muscle regeneration, and in muscle cell-specific experiments in vitro, alleviated the various symptoms caused by ischemic injury, including but not limited to, tissue necrosis, limb perfusion and vascular density, defective ischemic muscle regeneration, and limb muscle contractile function. In one embodiment of the present disclosure, Bag3 variant Ile81, which contains an isoleucine amino acid residue at position 81, and Bag3 variant Ile79, which contains an isoleucine amino acid residue at position 79 in humans, alleviated symptoms associated with ischemic injury in part through the regulation of ischemic myofiber regeneration and cellular autophagy.

Generally, there is a lack of effective medical therapies for ischemia, and surgical and percutaneous revascularization techniques frequently fail in subjects suffering from an ischemic injury, resulting in high mortality as well as amputation. Treatments involving BAG3^{Ile81}, as well as other candidate genes within Lsq-1 the QTL, would fill an important and unmet need. Moreover, such treatment is surprisingly effective in alleviating ischemic injury in a subject because it primarily targets the ischemic myocyte rather than, for example, the vasculature (i.e., therapeutic angiogenesis). As would be recognized by one of ordinary skill in the art, other gene and protein delivery modalities for treating and preventing ischemic injury by targeting the vasculature have not been successful. Pro-angiogenic approaches have been uniformly disappointing in their ability to alter outcomes in CLI patients. The advantages of BAG3^{Ile81} delivery over other current therapies includes its ability to induce a significant protective effect in ischemic muscle, thus preventing many of the manifestations of severe ischemia, before or after an ischemic insult is experienced.

Embodiments of the present disclosure include materials and methods to treat and prevent limb ischemia. In accordance with these embodiments, wildtype Bag3 and Bag3 variants, as well as BAG3 proteins/peptides encoded by wildtype Bag3 and Bag3 variants, include polypeptides encoding an isoleucine at position 79 of the human BAG3 protein (A79I). As further described herein (e.g., Example 14), wildtype Bag3 and Bag3 variants can be used to treat subjects having single nucleotide polymorphisms (SNPs) that have been associated with cardiac dysfunction (e.g., heart failure, and myofibrillar and cardiomyocyte myopathy), including but not limited to, a leucine at amino acid position 209 of the human BAG3 protein, an alanine at amino acid position 63 of the human BAG3 protein, and/or a serine at amino acid position 380 of the BAG3 protein. Other mutations or SNPs in Bag3 (known or not yet identified) that may contribute to one or more symptoms associated with an ischemic injury can also be targeted with the wildtype Bag3 and Bag3 variants, as disclosed herein.

Other Bag3 variants with therapeutic effects can include variants that encode BAG3 polypeptides having at least one other amino acid substitution at a position other than the amino acid substitutions at position 79 of human BAG3. Such Bag3 variants may enhance the therapeutic benefits of Bag3 when delivered to a subject suffering from an ischemic injury, as demonstrated in the present disclosure.

In some embodiments, effective delivery of wildtype Bag3, Bag3 variants, or BAG3 proteins/peptides encoded by wildtype Bag3 and/or Bag3 variants to a cell and/or tissue can be facilitated by various means, including, but not limited to, viral vectors, retroviral vectors, plasmid DNA, antisense RNA, peptide complexes (e.g., cationic peptides), lipids (e.g., lipofection, liposomes/micelles), small molecule targeting, micro/nano particles (e.g., exosomes) and the like. In some embodiments, wildtype Bag3, Bag3 variants, or BAG3 proteins/peptides encoded by wildtype Bag3 and/or Bag3 variants can be delivered to a cell and/or tissue using physical methods, including but not limited to, electroporation, microinjection, gene gun, impalefection, hydrostatic pressure, continuous infusion, sonication, and the like. In some embodiments, Bag3 and Bag3 variants can be delivered using a method that integrates Bag3 into a genome (genome editing), including but not limited to, CRISPR/Cas9, transcription activator-like effector nucleases (TALENs), zinc-finger nucleases, and the like. These and other methods can include delivery in vivo for the treatment and/or prevention of ischemic injury or one or more symptoms thereof in a subject such as a mammal (e.g., human patient). Delivery of Bag3 and Bag3 variants can also include in vitro or ex vivo, such as in cultured cells, tissues, and laboratory model organisms (e.g., mice, rats, zebrafish, fruit fly, worms, and the like).

In some embodiments, as is shown in the present disclosure, it is advantageous to deliver wildtype Bag3, Bag3 variants, or BAG3 proteins/peptides encoded by wildtype Bag3 and/or Bag3 variants using a virus-based approach, such as a targeting vector. For example, Bag3 and Bag3 variants can be delivered using retroviruses or adeno viruses, which include but are not limited to, adeno-associated viruses (AAVs), lentiviruses, pox viruses, alphaviruses, herpes viruses, and the like. These viruses can be pseudotyped (e.g., VSV G-pseudotyped lentivirus) in various ways that may confer different tropism for cells/tissues and can permanently or temporarily alter gene expression in an organism, as would be recognized by one of ordinary skill in the art based on the present disclosure.

Embodiments of the present disclosure include materials and methods to treat and prevent limb ischemia. In accordance with these embodiments, effective delivery of wildtype Bag3, Bag3 variants, or BAG3 proteins/peptides encoded by wildtype Bag3 and/or Bag3 variants to a cell and/or tissue can be facilitated using promoters, enhancers, and other DNA regulatory elements that confer tissue-specific and/or cell-specific expression. For example, Bag3 and Bag3 variants can be included in a targeting vector that contains one or more gene regulatory elements that confer specific expression in a muscle cell, a fibroblast, a stem cell, a pericyte, and an endothelial cell. In some embodiments, Bag3 and Bag3 variants can be expressed in muscle cells using muscle-specific regulatory elements such as muscle creatine kinase (MCK) promoters/enhancers, desmin (DES) promoters/enhancers, troponin I IRE (FIRE) promoters/enhancers, myosin light chain (MLC) promoters/enhancers, myosin heavy chain (MHC) promoters/enhancers, cardiac troponin C promoters/enhancers, troponin I promoters/enhancers, myoD gene family promoters/enhancers, actin alpha promoters/enhancers, actin beta promoters/enhancers, and actin gamma promoters/enhancers.

Embodiments of the present disclosure also include materials and methods to treat and prevent ischemia in neural tissue, such as ischemia related to a stroke, cerebral ischemia, or ischemia resulting from an injury/insult to the nervous system. In accordance with these embodiments, effective delivery of wildtype Bag3, Bag3 variants, or BAG3 proteins/peptides encoded by wildtype Bag3 and/or Bag3 variants to a cell and/or tissue of the nervous system (e.g., neurons, glial cells, oligodendrocytes, and neural crest cells) can be facilitated using promoters, enhancers, and other DNA regulatory elements that confer tissue-specific and/or cell-specific expression. For example, Bag3 and Bag3 variants can be included in a targeting vector that contains one or more gene regulatory elements that confer specific expression in a cell of the nervous system, including but not limited to, neurons, glial cells, oligodendrocytes, and neural crest cells. In some embodiments, Bag3 and Bag3 variants can be expressed in a cell of the nervous system using regulatory elements such as cytomegalovirus (CMV) promoters/enhancers, glial fibrillary acidic protein (GFAP) promoters/enhancers, synapsin I (SYN) promoters/enhancers, calcium/calmodulin-dependent protein kinase II promoters/enhancers, tubulin alpha I promoters/enhancers, and neuron-specific enolase and platelet-derived growth factor beta chain promoters/enhancers.

Embodiments of the present disclosure include materials and methods to treat and prevent limb ischemia. In accordance with these embodiments, effective delivery of wildtype Bag3, Bag3 variants, or BAG3 proteins/peptides encoded by wildtype Bag3 and/or Bag3 variants to a cell and/or tissue can treat or prevent an ischemic injury characterized as exhibiting necrosis, myopathy, and/or vascular deficiency. Treatment with Bag3 and Bag3 variants can lead to a reduction of one or more symptoms of ischemic injury such as necrosis, myopathy, and/or vascular deficiency. Treatment with Bag3 and Bag3 variants can also prevent one or more of these symptoms of ischemic injury from arising and/or worsening in a subject.

In some embodiments, administering wildtype Bag3, Bag3 variants, or BAG3 proteins/peptides encoded by wildtype Bag3 and/or Bag3 variants treats at least one symptom associated with ischemic injury by increasing one or more biological parameters, including but not limited to, muscle fiber cross-sectional area, capillary density, muscle function, muscle regeneration, stem cell activity, vascular density, and vascular luminal diameter. In some embodiments, administering wildtype Bag3, Bag3 variants, or BAG3 proteins/peptides encoded by wildtype Bag3 and/or Bag3 variants can increase myotube diameter, improve a myotube phenotype, improve contractile function, increase stem cell or satellite cell activity/myogenesis, increase mitochondrial number or respiratory function, increase autophagic flux, and/or decrease DNA fragmentation. Treatment with Bag3 and Bag3 variants can also prevent one or more these biological parameters of ischemic injury from arising and/or worsening in a subject. At the level of gene expression, administering wildtype Bag3, Bag3 variants, or BAG3 proteins/peptides encoded by wildtype Bag3 and/or Bag3 variants can treat at least one symptom associated with ischemic injury by causing increased expression of one or more of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker) and muscle RING-finger protein 1 (MuRF-1) or a decrease in expression of myostatin.

As would be recognized by one of ordinary skill in the art based on the present disclosure, ischemic injury can be caused by various disease indications, including but not limited to, peripheral artery disease comprising intermittent claudication or critical limb ischemia, muscular dystrophy, myofibrillar myopathy, degenerative myopathies, glycogen storage diseases, trauma, renal disease, atrial fibrillation, COPD, coronary artery disease, morbid obesity, cachexia, congestive heart failure, myocardial infarction, and diabetes mellitus. Treatment of these disease indications can include administering pharmaceutical compositions containing wildtype Bag3, and/or Bag3 variants (e.g., A79I), as well as BAG3 proteins/peptides encoded by wildtype Bag3 and Bag3 variants, as described herein. Pharmaceutically acceptable compositions can include one or more polynucleotide constructs that include either wildtype Bag3 or a Bag3 variant(s), and pharmaceutically acceptable compositions can include one or more polynucleotide constructs that include both wildtype Bag3 and a Bag3 variant(s). Pharmaceutically acceptable compositions can also include one or more wildtype BAG3 proteins/peptides and/or fragments thereof, one or more BAG3 variant proteins/peptides and/or fragments thereof, or both. Pharmaceutically acceptable compositions can include these various Bag3 constructs and peptides with one or more adjuvants, excipients, carriers, buffers, diluents, and/or other customary pharmaceutical auxiliaries. In some embodiments, the present disclosure provides pharmaceutical compositions that include pharmaceutically acceptable salts or derivatives thereof, together with one or more pharmaceutically acceptable carriers, and optionally, other therapeutic and/or prophylactic ingredients. Suitable carrier(s) are generally compatible with the other ingredients of the formulation and not harmful to the recipient thereof.

Pharmaceutically acceptable compositions that include wildtype Bag3, Bag3 variants, or BAG3 proteins/peptides encoded by wildtype Bag3 and/or Bag3 variants can be administered as part of a clinically appropriate treatment and dosing regimen. For example, pharmaceutically acceptable compositions containing wildtype Bag3, Bag3 variants, or BAG3 proteins/peptides encoded by wildtype Bag3 and/or Bag3 variants can be administered in single dose or multiple doses by means such as intramuscular injection, percutaneous injection, intraperitoneal injection, intravenous injection, and oral consumption/ingestion. Pharmaceutically acceptable compositions containing wildtype Bag3, Bag3 variants, or BAG3 proteins/peptides encoded by wildtype Bag3 and/or Bag3 variants can be administered in a single dose or multiple doses over the course of a day, week, month, or year, depending on various factors such as the disease indication being treated, the subject's medical needs/history, and the type of formulation. In some embodiments, pharmaceutically acceptable compositions containing wildtype Bag3, Bag3 variants, or BAG3 proteins/peptides encoded by wildtype Bag3 and/or Bag3 variants can be administered to a subject in two or more separate injections (e.g., 3 injections) within a single day or within a week. In some embodiments, pharmaceutically acceptable compositions containing wildtype Bag3, Bag3 variants, or BAG3 proteins/peptides encoded by wildtype Bag3 and/or Bag3 variants can be administered to a subject before an ischemic injury or insult has taken place to prevent one or more symptoms of the ischemic injury from arising; or a pharmaceutically acceptable compositions wildtype Bag3, Bag3 variants, or BAG3 proteins/peptides encoded by wildtype Bag3 and/or Bag3 variants can be administered to a subject after an ischemic injury or insult has taken place to prevent worsening of one or more symptoms of the ischemic injury.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions are typically be sterile and stable under the conditions of manufacture and storage.

In some embodiments, pharmaceutically acceptable compositions containing wildtype Bag3, Bag3 variants, or BAG3 proteins/peptides encoded by wildtype Bag3 and/or Bag3 variants can be administered as part of a kit. For example, pharmaceutically acceptable compositions containing wildtype Bag3, Bag3 variants, or BAG3 proteins/peptides encoded by wildtype Bag3 and/or Bag3 variants can be administered to a subject via an injection using an injection device, such as a needle and syringe, as would be readily recognized by one of ordinary skill in the art based on the present disclosure.

In some cases, the methods and materials described herein may be used in subjects with less severe ischemia, such as in human patients with intermittent claudication, as the effects of BAG3^{Ile79} gene delivery appear to enhance vascularity and tissue perfusion. Thus, improved vascularity would be predicted to prevent ischemia-induced morbidity (e.g., severe pain with exertion). Given the effects of BAG3^{Ile79} on skeletal muscle myocytes and blood vessels, other conditions in which skeletal muscle function is adversely affected might be amenable to treatment, including, but not limited to, muscular dystrophy, congestive heart failure, and diabetes mellitus, as well as any disease characterized by skeletal myopathy. Moreover, BAG3 is expressed in the myocardium as well as in skeletal muscle, and it is possible that treatment of ischemic myocardium with this approach could prevent complications of myocardial ischemia, such as myocardial infarction and congestive heart failure.

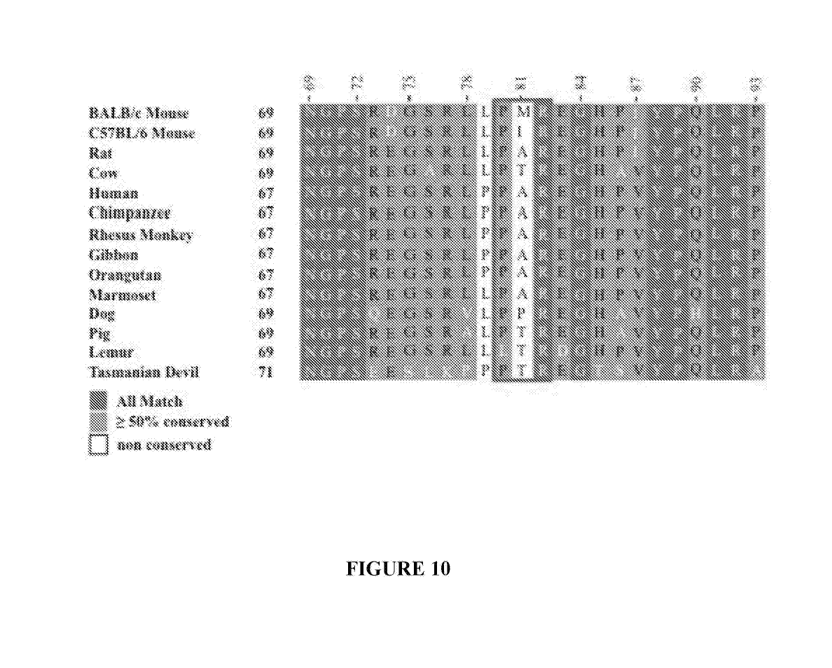

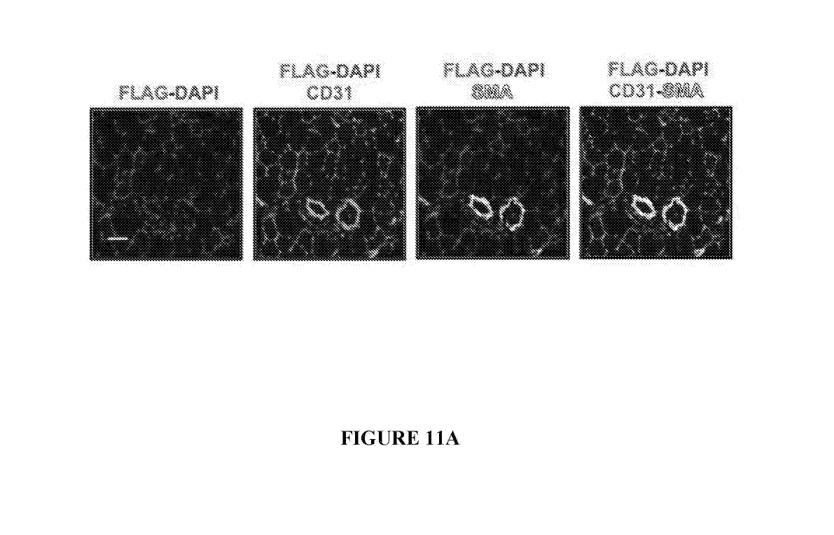

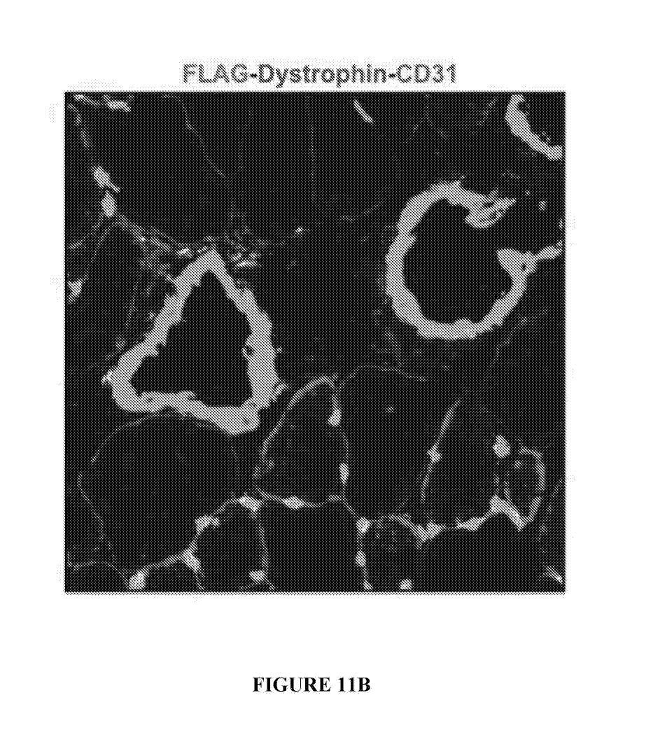

In some embodiments of the present disclosure, a single coding polymorphism in a gene within the Lsq-1 QTL, Bag3, can partially determine susceptibility to skeletal muscle tissue necrosis following HLI in mice. An isoleucine to methionine variant at position 81 in the murine BAG3 protein can be sufficient to confer susceptibility to necrosis and myopathy, two hallmarks of ischemic injury. However, the variant of BAG3 that comprises an isoleucine at position 81 (SEQ ID NO:1) can confer a protective effect that can rescue the detrimental symptoms of ischemic injury. As shown in FIG. 10, this position of variation occurs at amino acid position 79 in human BAG3 (SEQ ID NO:2); thus, in some embodiments, an alanine to isoleucine amino acid substitution at position 79 of human BAG3 can confer a protective effect that can rescue the detrimental symptoms of ischemic injury in humans. As further disclosed herein, wildtype BAG3, as well as other BAG3 protein variants in humans can confer a protective effect that can rescue the detrimental symptoms of ischemic injury in humans, including, for example, humans with known Bag3 SNPs, such as a leucine at amino acid position 209 of the human BAG3 protein, an alanine at amino acid position 63 of the human BAG3 protein, and/or a serine at amino acid position 380 of the BAG3 protein. Other mutations or SNPs in Bag3 (known or not yet identified) that may contribute to one or more symptoms associated with an ischemic injury can also be targeted with the wildtype Bag3 and Bag3 variants, as disclosed herein.

Accordingly, the present disclosure demonstrates that BALB/c mice congenic for a fragment of the BL6 Lsq-1 QTL that contained the isoleucine variant of Bag3 were resistant to ischemic tissue necrosis, displaying enhanced myofiber integrity and regeneration after HLI as well as increased vascular density. Other genes with a variety of known and putative functions are contained within Lsq-1, and may have similar protective effects. It some cases, the genes within Lsq-1 may all contribute somewhat to the limb necrosis phenotype observed in BALB/c mice after HLI. For example, Adam 12, a gene within Lsq-1 that has also been linked to skeletal muscle regeneration, was recently shown to be differentially expressed in BL6 and BALB/c mice and to regulate outcomes after HLI.

The importance of BAG3 in the regulation of ischemic muscle cell survival was demonstrated by the significant beneficial effects of the BL6 variant, BAG3^{Ile81}, when exogenously expressed in BALB/c muscle, as shown in the present disclosure. These results are surprising and would appear to contrast with prior observations in which Bag3 heterozygous knockout mice displayed no differences in recovery from HLI compared to their wild-type littermates. Additionally, postnatal day 7 Bag3^{-/-} pups were shown to have no differences in pial collateral artery number or diameter, which suggests that a primary effect of BAG3 is on skeletal muscle cells and not the vasculature. This underscores the surprising effects BAG3 was shown in the present disclosure to have on ischemic injury, which includes alleviating various vascular symptoms. Consistent with this is the finding described herein in which AAV6-mediated expression of BAG3 was localized to muscle cells and not endothelial cells. Also, BAG3^{Ile81} but not BAG3^{Met81} altered skeletal myoblast fusion and regeneration, which likely contributed to the beneficial effects on ischemic muscle survival. Furthermore, the present disclosure shows that BAG3^{Ile81} had similar effects on muscle regeneration after cardiotoxin injection, a non-ischemic muscle injury model. Taken together, these findings demonstrate an important functional difference in the two BAG3 variants.

Embodiments of the present disclosure also demonstrate that capillary density was increased in mice treated with AAV-BAG^{Ile81}. Although AAV was efficiently expressed in myofibers, no immunofluorescence co-labeling of CD31⁺, SMA⁺, and FLAG tagged construct was observed, suggesting that the AAV6 serotype may have injected more efficiently and/or was expressed more robustly in muscle cells in vivo. It is also possible that improved muscle cell survival resulted in enhanced ischemia-induced expression of vascular growth factors and subsequent angiogenesis.

Embodiments of the present disclosure demonstrate that Bag3 is an important component of the Lsq-1 QTL regulation of ischemic injury. For example, the murine model of HLI is characterized by rapid onset of tissue ischemia, which contrasts with clinical PAD, which develops gradually over the course of years as a result of chronic atherosclerosis. Despite this difference, clinical CLI is characterized by marked tissue injury similar to that observed in the acute HLI model, thus the present disclosure strongly support Bag3 as a candidate for the regulation of tissue injury and recovery during PAD. Overall, these findings are an important initial step in understanding the complex multi-tissue pathology of CLI, and they provide critical insights into genetic determinants that may lead to diagnostic and therapeutic interventions for diseases associated with ischemic injury.

### EXAMPLES

The following examples are illustrative of disclosed methods. In light of the present disclosure, those of skill in the art will recognize that variations of these examples and other examples of the disclosed method would be possible without undue experimentation.

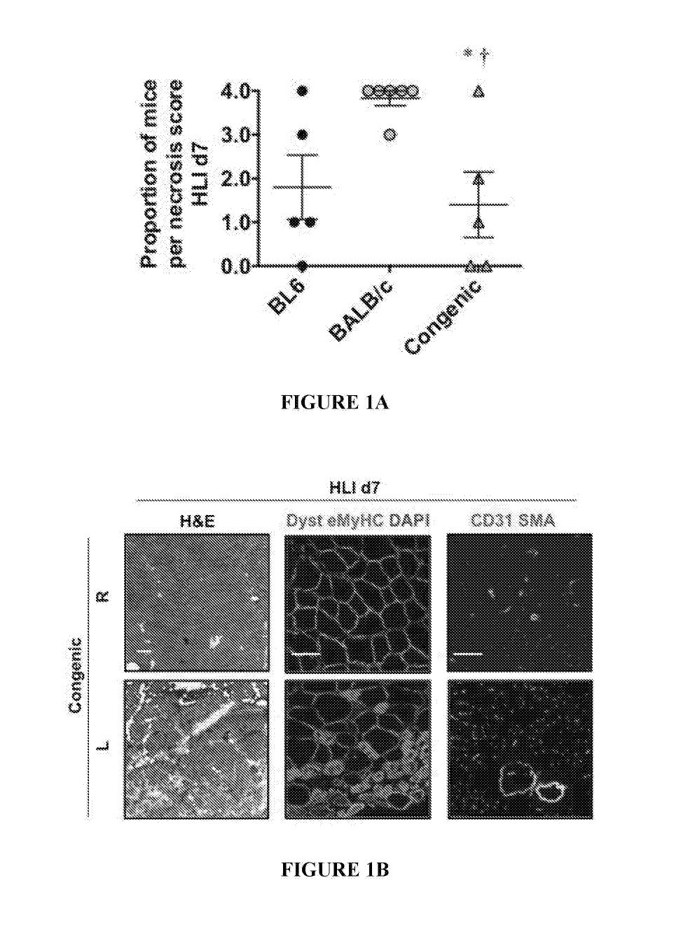

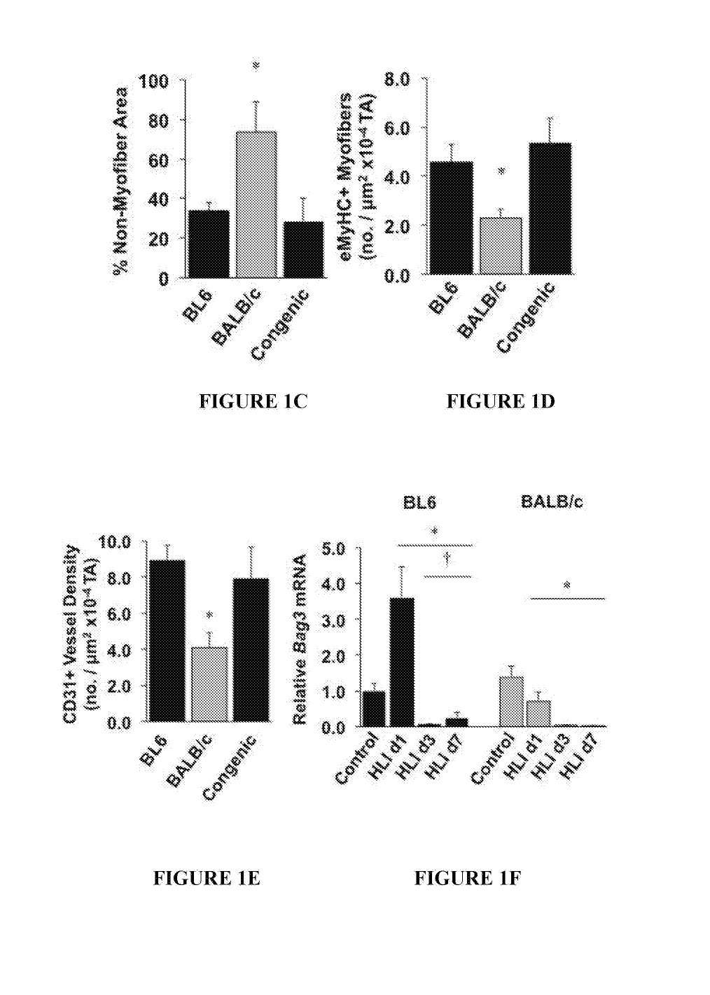

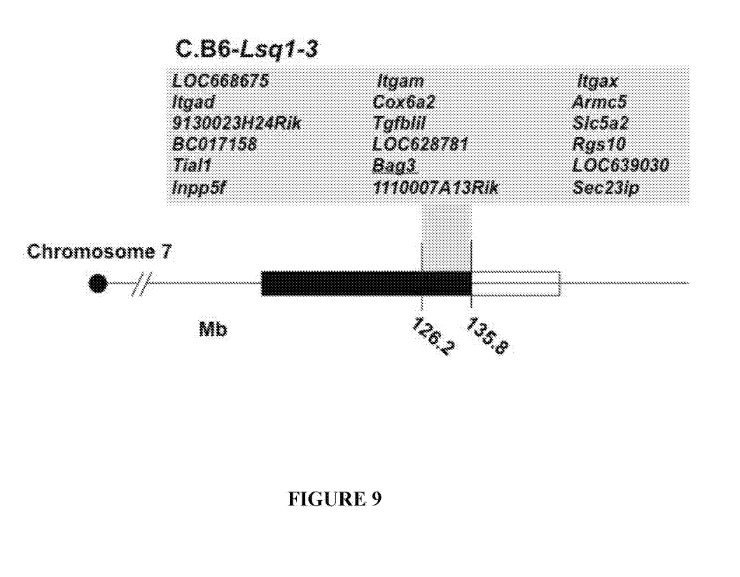

Example 1: Identification of Bag3 as a potential protective gene in ischemic tissue necrosis. C57BL/6xBALB/c offspring were bred to parental BALB/c mice to generate a line of BALB/c mice (C.B6-Lsq1-3) congenic for a ˜12.06 Mb region of BL6 chr. 7 (Congenic) that includes Bag3 but excludes 19 other genes from Lsq-1 (FIG. 9). This line is the same as the previously reported C.B6-Civq1-3. These mice were subjected to HLI alongside parental BL6 and BALB/c mice, and necrosis scores were determined 7 days later (FIG. 1A). The degree of tissue necrosis in Congenic mice mirrored that of parental BL6 mice, indicating that BL6-encoded variants in this region contributed to muscle survival. Histological analysis of ischemic limb muscle largely revealed a restoration of ischemic muscle (L) fascicular and vascular morphology compared to the contralateral limb (R) (FIG. 1B), which was previously observed in BL6 but not BALB/c mice. Myofiber integrity (non-myofiber area), regeneration (embryonic MyHC staining), and capillary density (CD31 staining) were all similar in the ischemic limbs of Congenic and BL6 mice but were significantly different from those in BALB/c mice (FIGS. 1C-1F). Bag3 mRNA expression was significantly greater in BL6 mice on day 1 after HLI (FIG. 1G), a time when limb perfusion is comparable between the strains. Together these data support that BAG3, as a member of Lsq-1, plays a role in ischemic muscle survival.

As shown in FIG. 10, the sequence of BAG3 protein across various species reveals a lack of conservation at amino acid residue 81 but a high degree of conservation among surrounding residues. The full length mouse BAG3 polypeptide sequence is represented as:

(SEQ ID NO: 1)
MSAATQSPMMQMASGNGASDRDPLPPGWEIKIDPQTGWPFFVDHNSRTTT
WNDPRVPPEGPKDTASSANGPSRDGSRLLPIREGHPIYPQLRPGYIPIPV
LHEGSENRQPHLFHAYSQPGVQRFRTEAAAATPQRSQSPLRGGMTEAAQT
DKQCGQMPATATTAAAQPPTAHGPERSQSPAASDCSSSSSSASLPSSGRS
SLGSHQLPRGYIPIPVIHEQNITRPAAQPSFHQAQKTHYPAQQGEYQPQQ
PVYHKIQGDDWEPRPLRAASPFRSPVRGASSREGSPARSGTPVHCPSPIR
VHTVVDRPQPMTHREPPPVTQPENKPESKPGPAGPDLPPGHIPIQVIRRE
ADSKPVSQKS
PPPAEKVEVKVSSAPIPCPSPSPAPSAVPSPPKNVAAEQKAAPSPAPAEP
AAPKSGEAETPPKHPGVLKVEAILEKVQGLEQAVDSFEGKKTDKKYLMIE
EYLTKELLALDSVDPEGRAD
VRQARRDGVRKVQTILEKLEQKAIDVPGQVQVYELQPSNLEAEQPLQEIM
GAVVADKDKKGPENKDPQTESQQLEAKAATPPNPSNPADSAGNLVAP.

The full length human BAG3 polypeptide sequence is represented as:

(SEQ ID NO: 2)
MSAATHSPMMQVASGNGDRDPLPPGWEIKIDPQTGWPFFVDHNSRTTTWN
DPRVPSEGPKETPSSANGPSREGSRLPPAREGHPVYPQLRPGYIPIPVLH
EGAENRQVHPFHVYPQPGMQRFRTEAAAAAPQRSQSPLRGMPETTQPDKQ
CGQVAAAAAAQPPASHGPERSQSPAASDCSSSSSSASLPSSGRSSLGSHQ
LPRGYISIPVIHEQNVTRPAAQPSFHQAQKTHYPAQQGEYQTHQPVYHKI
QGDDWEPRPLRAASPFRSSVQGASSREGSPARSSTPLHSPSPIRVHTVVD
RPQQPMTHRETAPVSQPENKPESKPGPVGPELPPGHIPIQVIRKEVDSKP
VSQKPPPPSEKVEVKVPPAPVPCPPPSPGPSAVPSSPKSVATEERAAPST
APAEATPPKPGEAEAPPKHPGVLKVEAILEKVQGLEQAVDNFEGKKTDKK
YLMIEEYLTKELLALDSVDPEGRADVRQARRDGVRKVQTILEKLEQKAID
VPGQVQVYELQPSNLEADQPLQAIMEMGAVAADKGKKNAGNAEDPHTETQ
QPEATAAATSNPSSMTDTPGNPAAP.

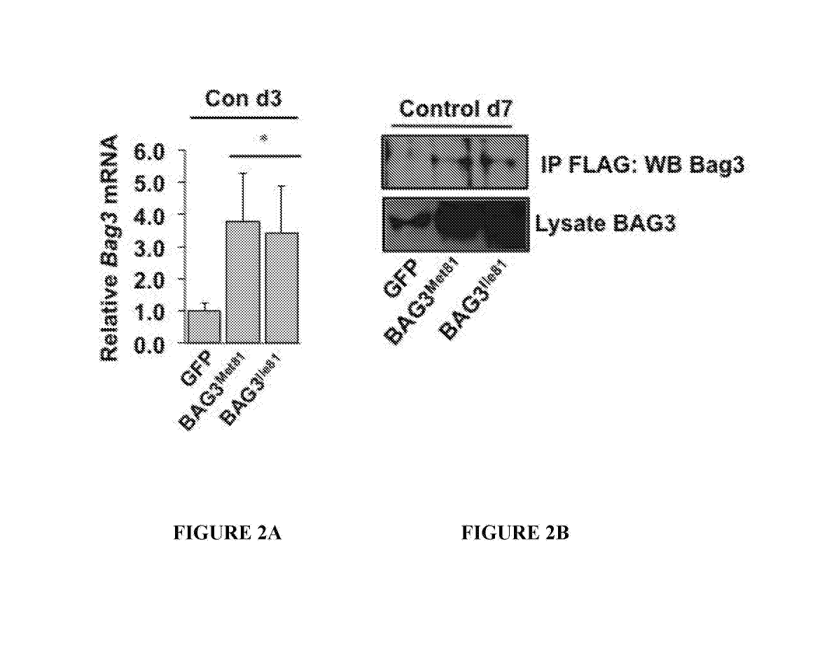

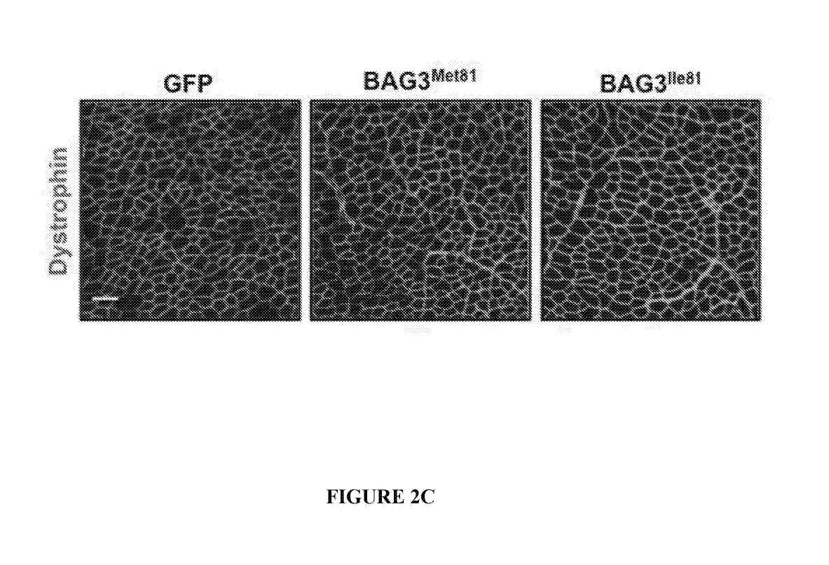

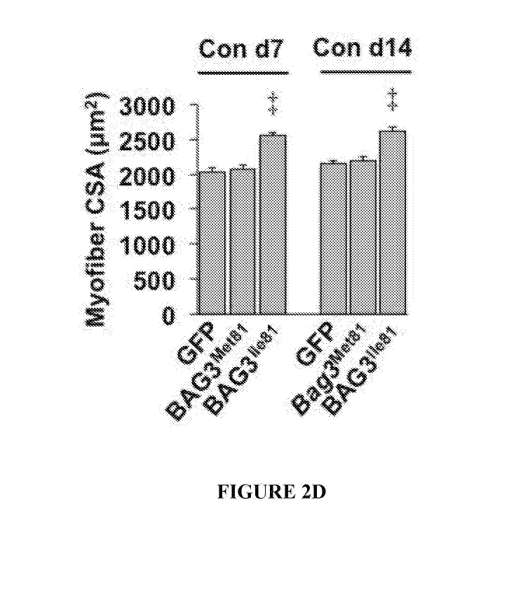

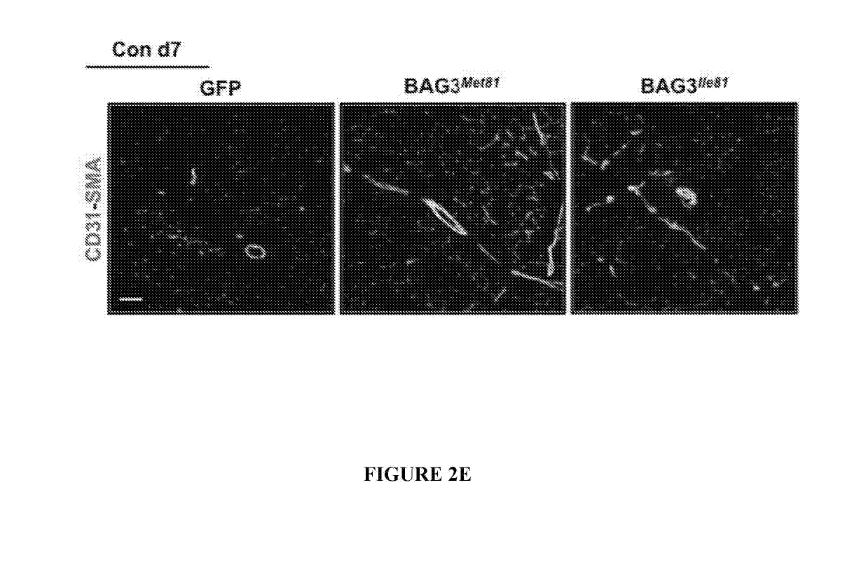

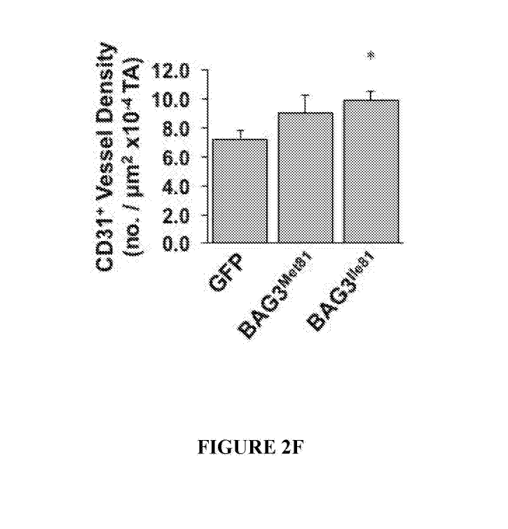

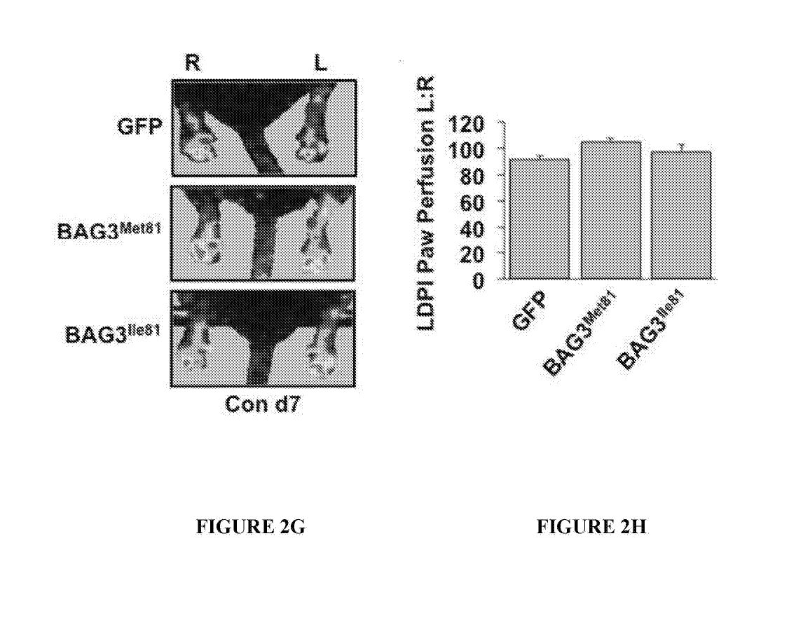

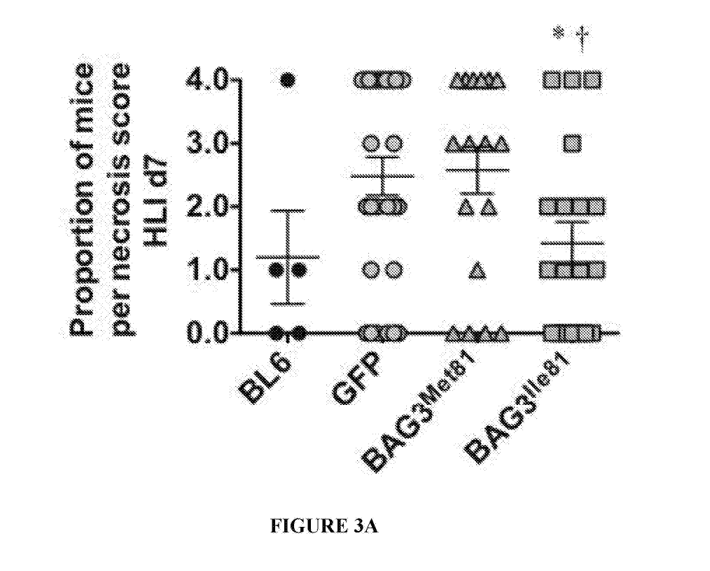

BAG3^{Ile81} gain of function induces BALB/c muscle hypertrophy and vascular expansion. Sequencing of the coding region and known regulatory elements of Bag3 revealed an isoleucine to methionine change at residue 81 (181M), a position with little conservation among a number of mammalian species despite a high degree of similarity of surrounding residues (FIG. 10). To test whether gain of function of either variant would alter the basal muscle morphology or vascular profile, serotype 6 adeno-associated viruses (AAV6) encoding either variant of BAG3 with a FLAG epitope tag was injected into both the TA and gastrocnemius (Gastroc) muscles of BALB/c mice. AAV6-BAG3 expression was verified in vivo by immunofluorescence (IF) microscopy for FLAG (FIG. 11). In BALB/c muscles, mRNA (FIG. 2A) and protein (FIG. 2B) of the two variants were expressed at equal levels in non-ischemic muscle. The BL6 variant (BAG3^{Ile81}) slightly but significantly increased TA myofiber size (FIGS. 2C-2D) and muscle CD31⁺ vessel density (FIGS. 2E-2F). Despite these vascular changes, LDPI-measured perfusion was not affected by either variant at baseline (FIGS. 2G-2H).

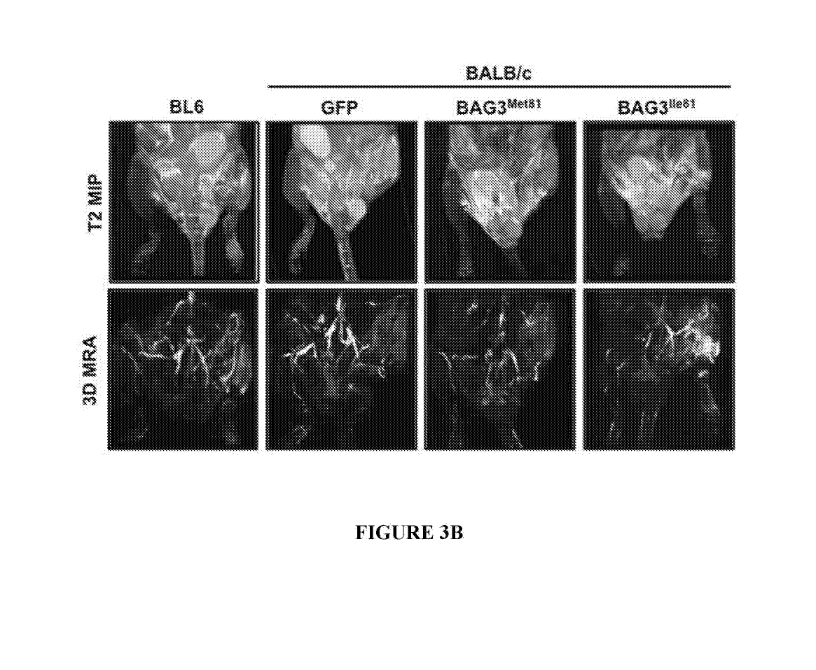

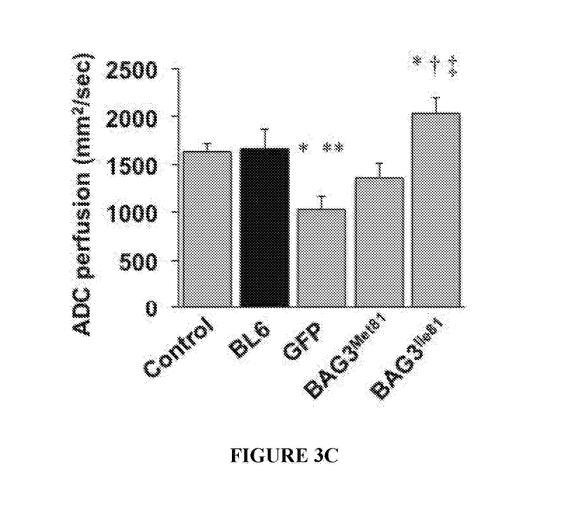

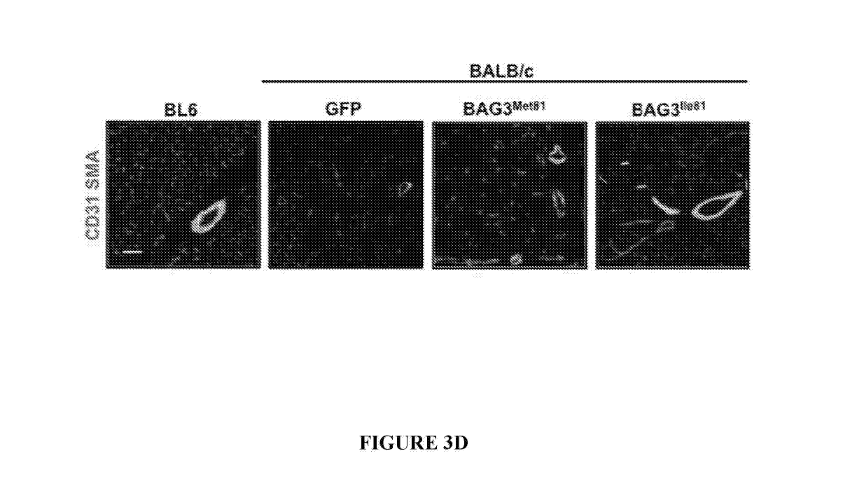

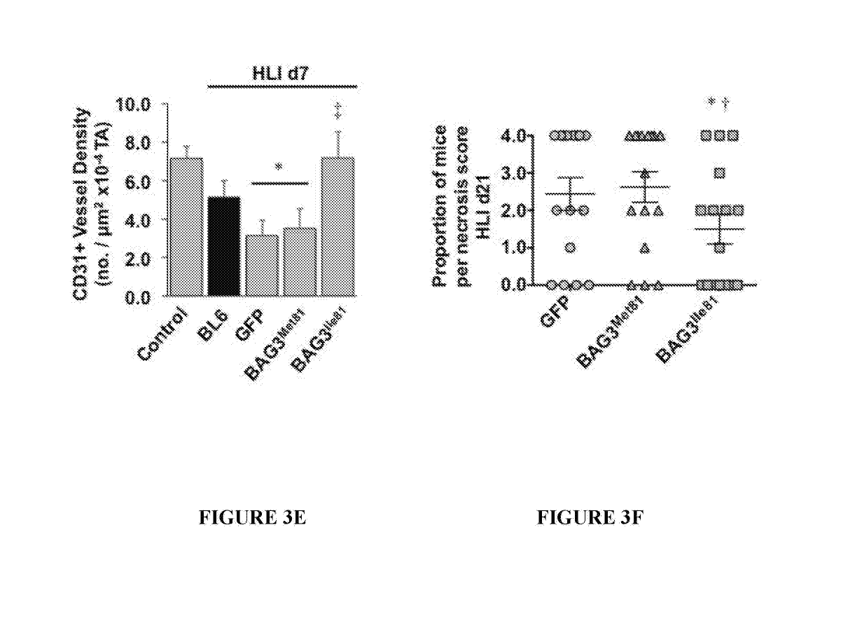

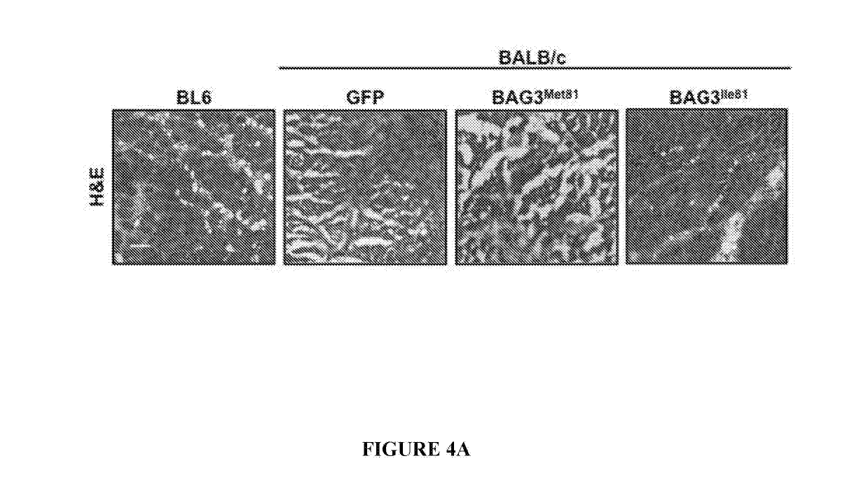

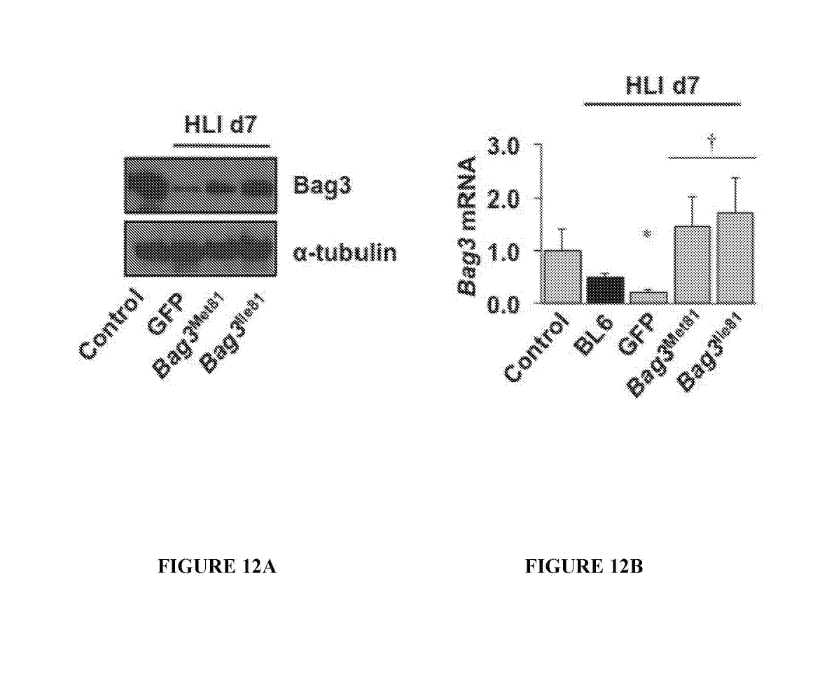

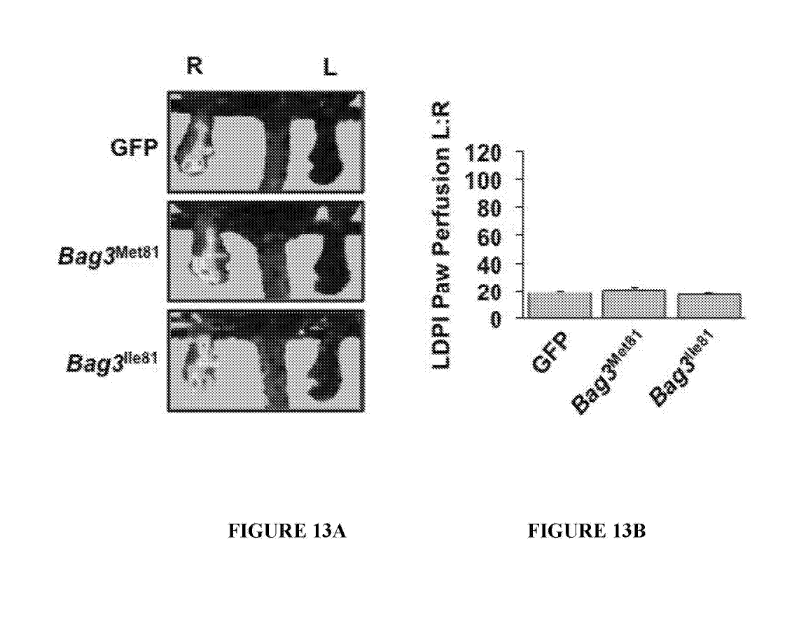

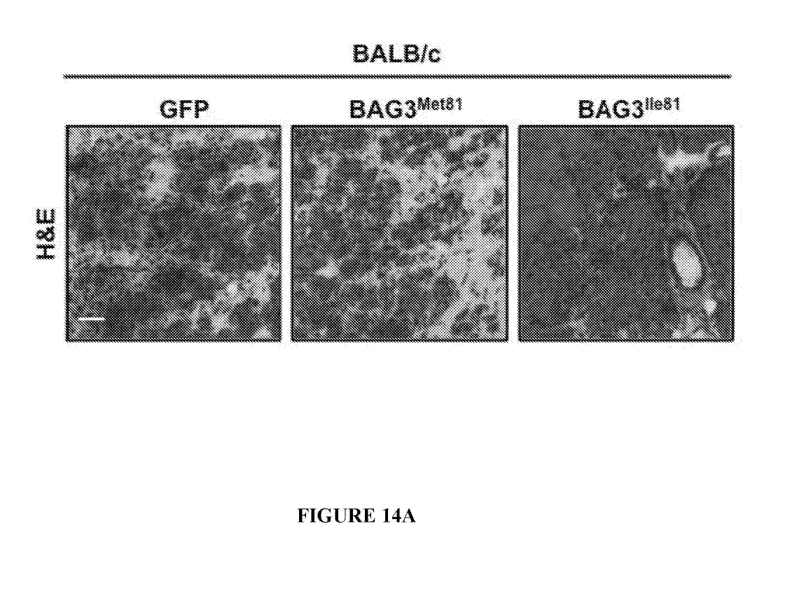

Example 2: The BL6 BAG3 variant, BAGIle81, prevents ischemic limb necrosis in BALB/c mice. Whether the BL6 variant, BAG3^{Ile381}, could protect against ischemic necrosis in BALB/c mice was investigated. Equal expression levels of the two variants after HLI was confirmed (FIG. 12). Notably, injection of either variant rescued the ischemia-induced loss of BAG3 protein and Bag3 mRNA but did not result in significant overexpression compared to baseline. Strikingly, expression of BAG3^{Ile81} in BALB/c mice conferred significant protection against ischemic tissue necrosis (FIG. 3A). Similar perfusion deficits immediately post HU surgery using LDPI were verified (FIG. 13), but because the susceptibility to ischemic tissue necrosis has been linked to tissue perfusion, magnetic resonance angiography (MRA) was used to examine limb muscle perfusion. Expression of BAG3^{Ile81}, but not BAG3^{Met81}, significantly increased vascular volume and apparent diffusion coefficient (ADC) perfusion (FIGS. 3B-3C) in the ischemic limb. To assess the vascular effects of BAG3^{Ile81}, IF for vascular markers was performed (FIG. 3D). On day 7 post-HLI, CD31⁺ capillary density decreased significantly in BALB/c mice expressing GFP or BAG3^{Met81} compared to BL6, whereas BAG3^{Ile81} expression rescued capillary density to levels that were not different from those in ischemic BL6 mice (FIG. 3E). Importantly, expression of BAG3^{Ile81} in BALB/c mice conferred protection against necrosis up to 21 days after HLI (FIG. 3F).

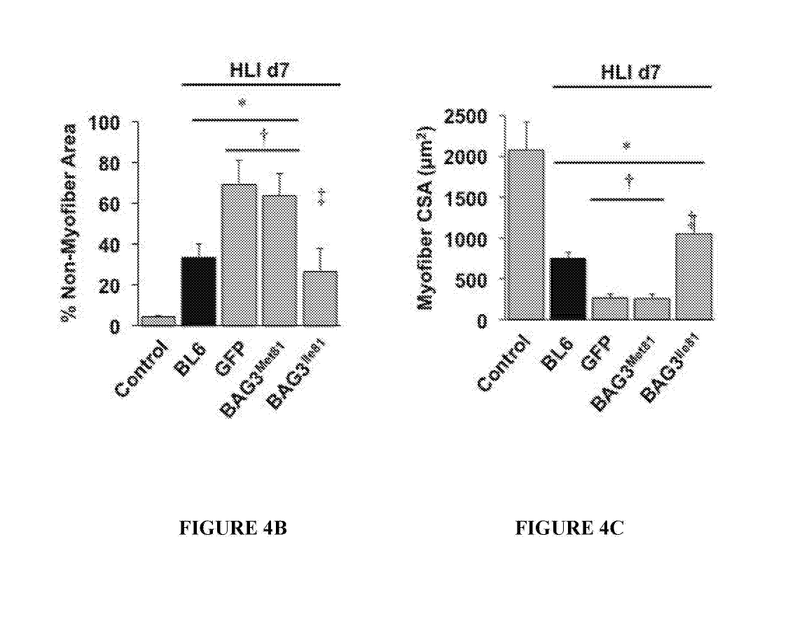

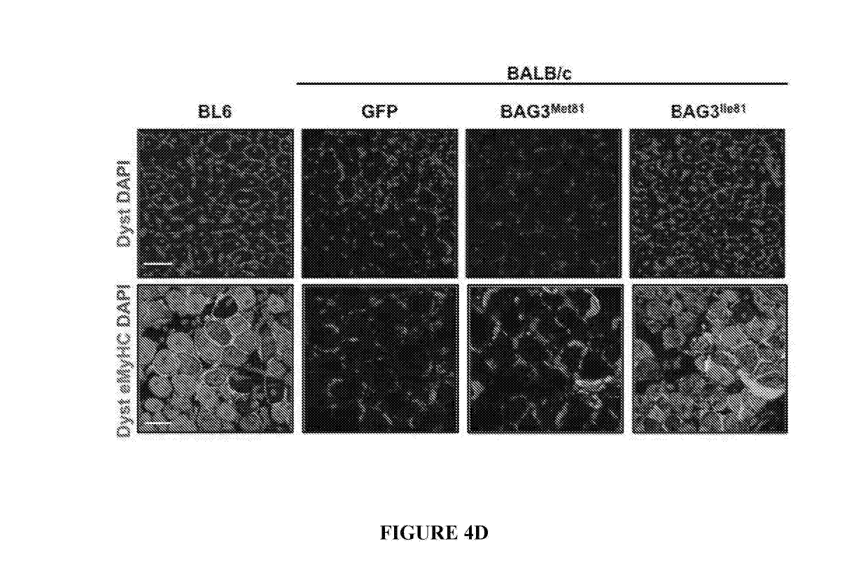

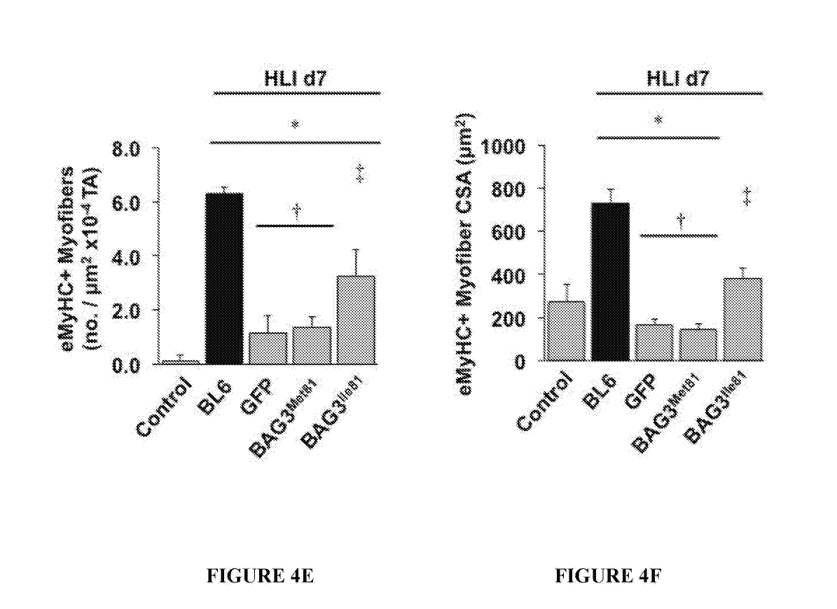

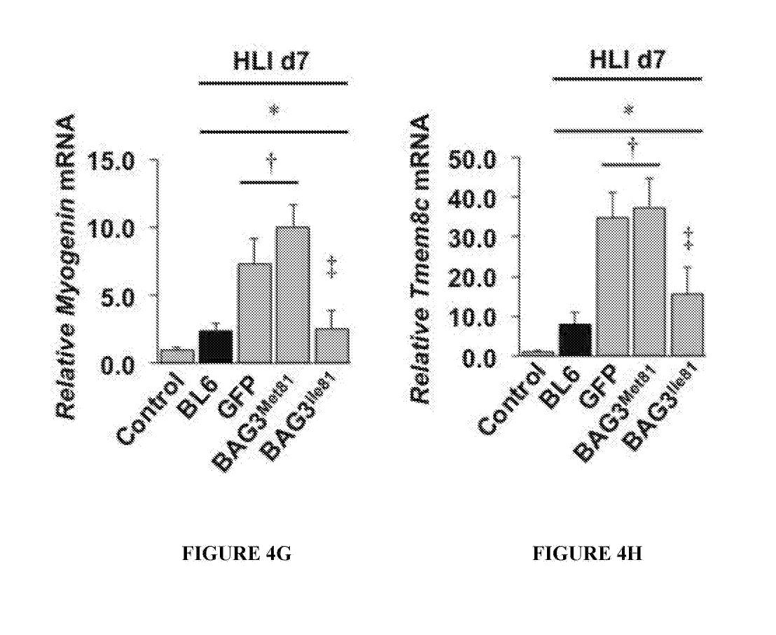

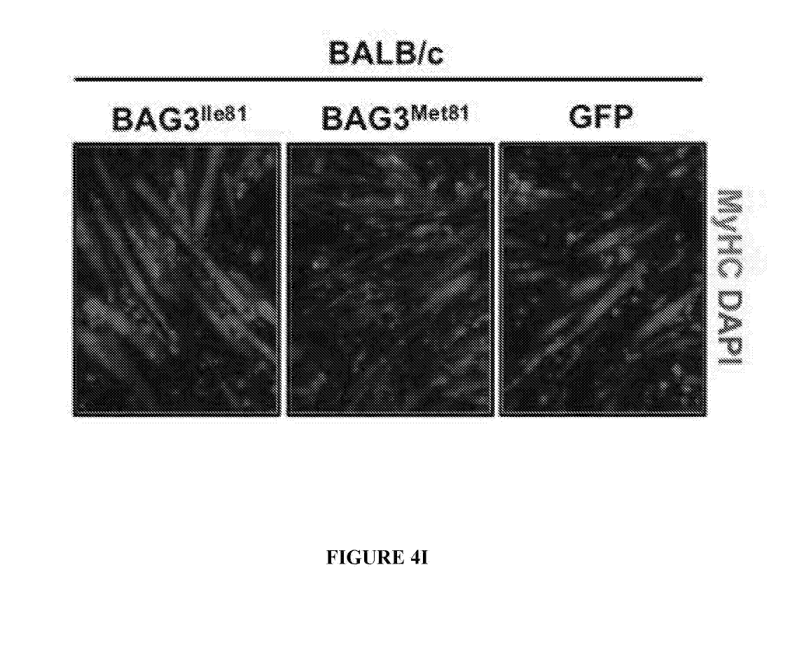

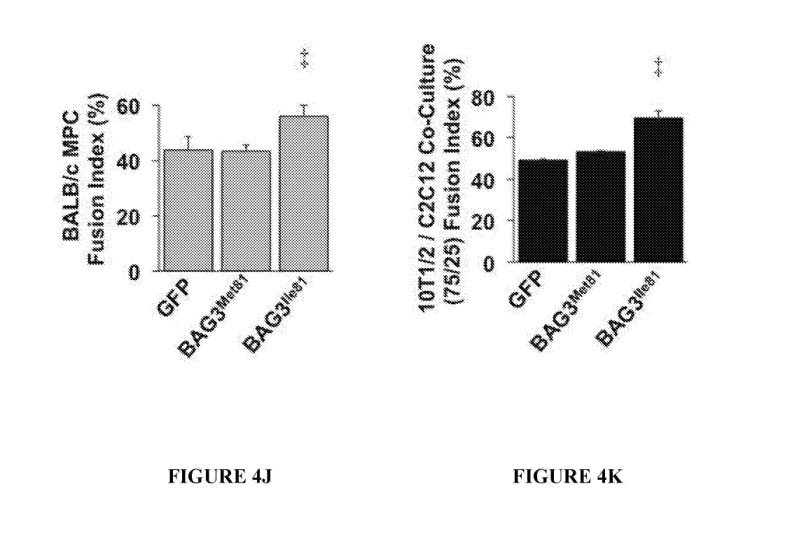

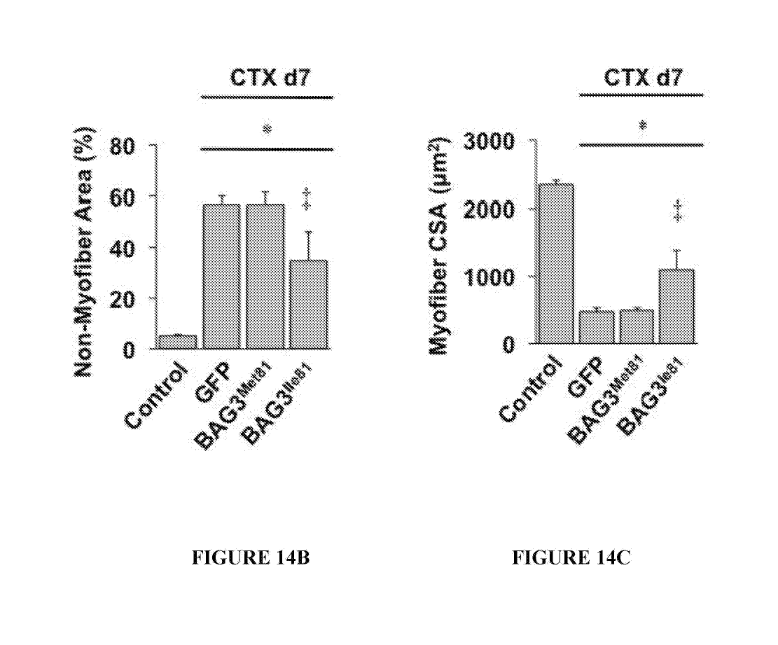

Example 3: BAG3Ile81 promotes muscle regeneration by enhancing myofiber differentiation and muscle precursor cell fusion. Bag3 null mice and humans with certain BAG3 mutations undergo marked skeletal muscle degeneration with a failed regenerative response. This phenotype is similar to that observed in ischemic BALB/c mice, suggesting that the BAG3 variants might differentially regulate muscle regeneration. Ischemic limb muscle from BAG3^{Ile81}-expressing BALB/c mice appeared morphologically similar to that in BL6 mice (FIG. 4A) and quantitatively displayed similar non-myofiber area (FIG. 4B) and intact myofiber cross-sectional area (FIG. 4C), consistent with either protection from ischemic injury or an improved regenerative response. To test this, myofiber regeneration was quantified after AAV delivery and HLI. The number of eMyHc⁺ myofibers was reduced in GFP- and BAG3^{Met81}-expressing mice compared to BL6 mice, and expression of BAG3^{Ile81} rescued this deficit (FIGS. 4D-4E). eMyHC⁺ myofibers expressing either GFP or BAG3^{Met81} were also smaller than those expressing BAG3^{Ile81} (FIG. 4F). To determine whether Bag3 variation has a general impact on BALB/c skeletal muscle regeneration, the BAG3 variants' effects following Naja nigricolis venom injection (a commonly used model of skeletal muscle regeneration) were tested. Similar to aforementioned results in the HLI model, resolution of muscle injury was accelerated in BALB/c mice expressing BAG3^{Ile81} compared to GFP or BAG3^{Met81} (FIG. 14), as demonstrated by a significant reduction in non-contractile tissue and greater myofiber size. Taken together, these results suggest that the BAG3^{Ile81} variant promotes muscle regeneration in different models of muscle injury, and it confers dominant protection in BALB/c mice, which endogenously express BAG3^{Met81}.

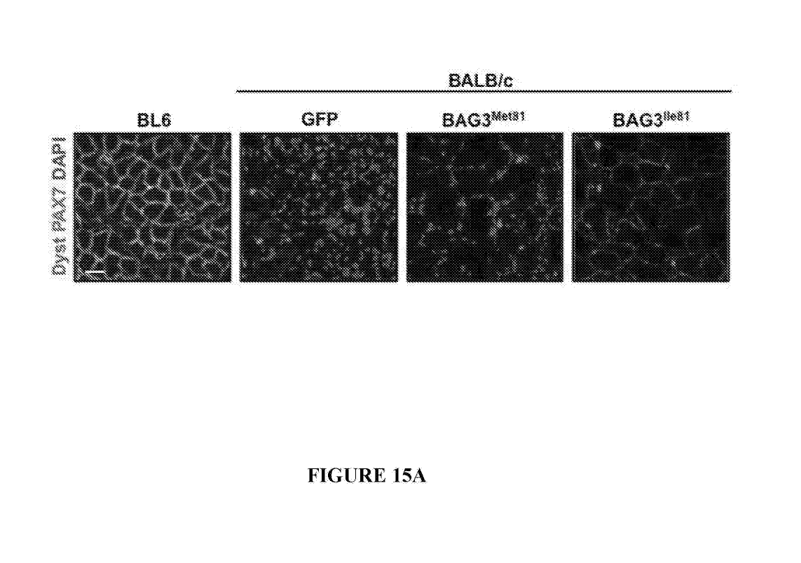

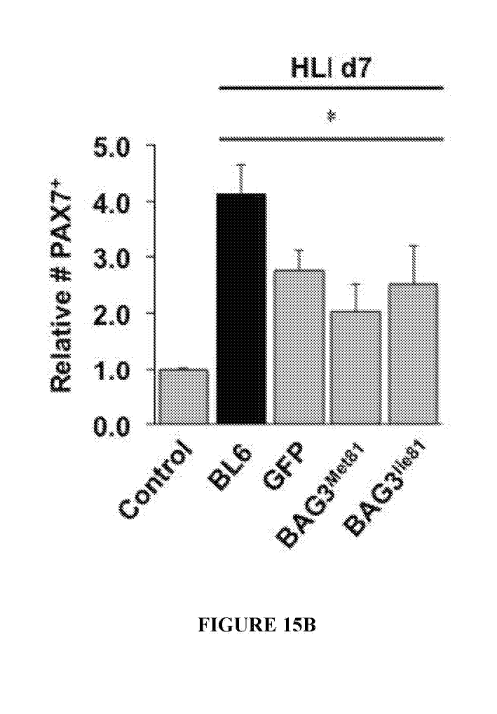

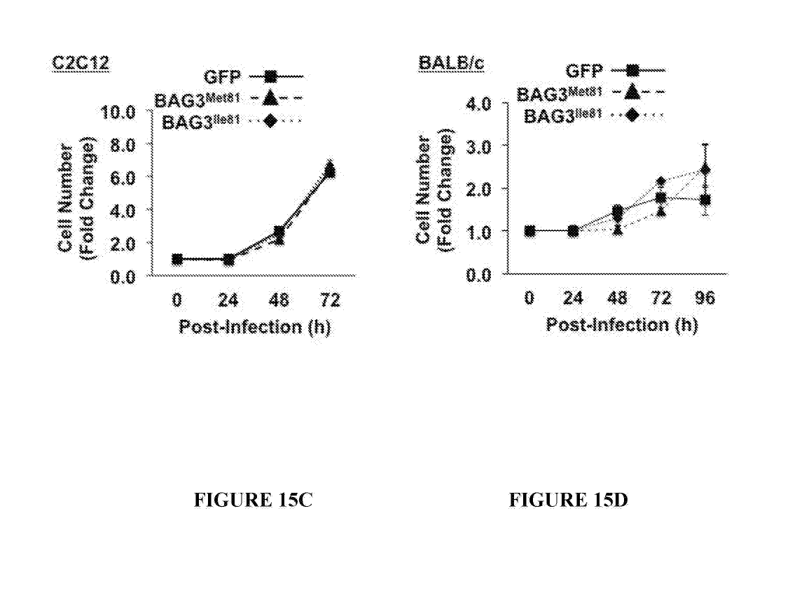

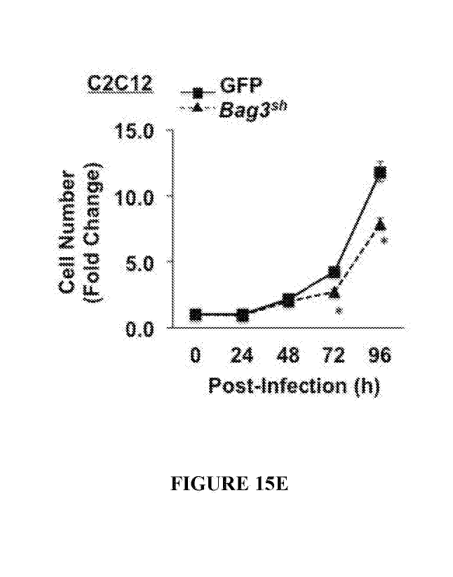

Muscle regeneration depends somewhat on the number, function, and proliferation of PAX7⁺ satellite cells. Compared to non-ischemic BALB/c muscle, PAX7⁺ nuclei increased in all groups after HLI, and no significant differences were noted among BAG3 variants (FIGS. 15A-15B), indicating that HLI induces the PAX7⁺ muscle cell proliferative response independent of the BAG3 variant. These findings were confirmed in vitro in C2C12 (FIG. 15C) and primary BALB/c myoblasts (FIG. 15D), in which expression of either BAG3 variant or GFP had no effect on skeletal myoblast proliferation. Stable silencing of BAG3 expression in C2C12 myoblasts using retroviral shRNA resulted in a slight but significant decrease in cell number after 72 and 96 hours of proliferation (FIG. 15E), however this effect was due to increased cell death accompanying BAG3 knockdown (not shown), which has been demonstrated previously.

Muscle regeneration also depends on precursor cell differentiation and fusion with existing myofibers. BAG3^{Ile81} expression restored the mRNA expression of the myogenic regulatory factor myogenin (FIG. 4G) and the differentiation/fusion proponent Tmem8c (myomaker; FIG. 4H) to BL6 levels during ischemia, consistent with improved differentiation and fusion capacity. In vitro, the fusion of BALB/c primary myoblasts into myotubes was significantly increased by BAG3^{Ile81} (FIGS. 4I-4J). Furthermore, myotube formation by C2C12 myoblasts co-cultured with a predominance of 10T1/2 pluripotent pericytes was improved by BAG3^{Ile81} (FIG. 4K). Taken together, these results demonstrate that BAG3^{Ile81} promotes ischemic muscle regeneration in part by enhancing muscle precursor cell differentiation and fusion.

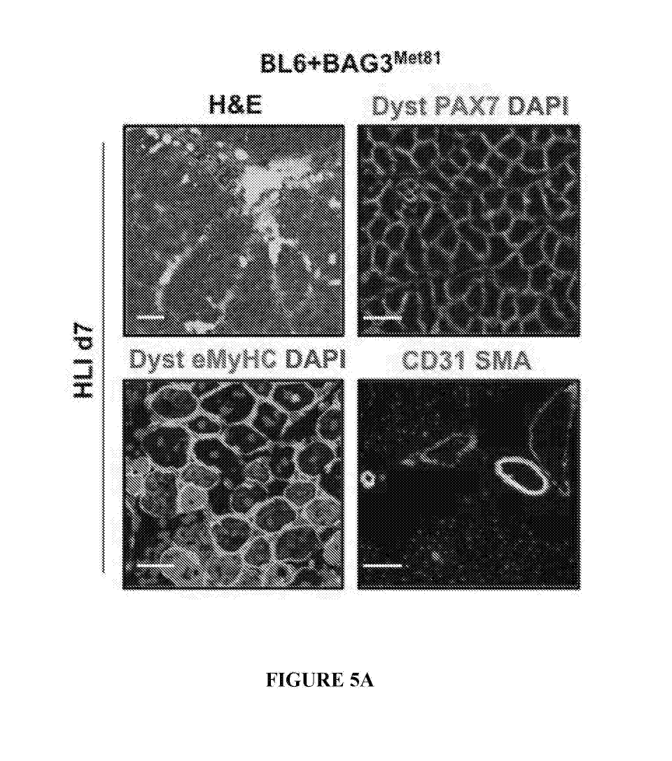

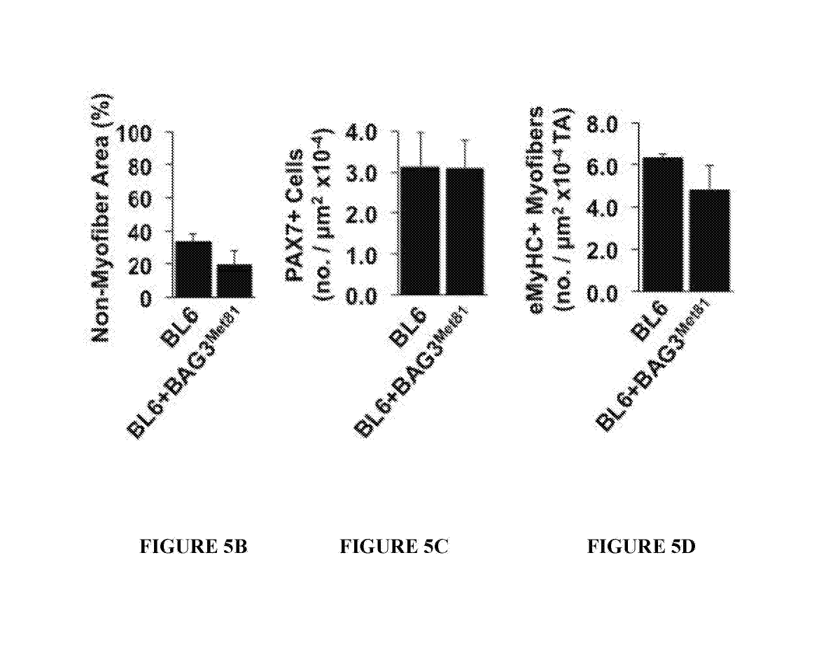

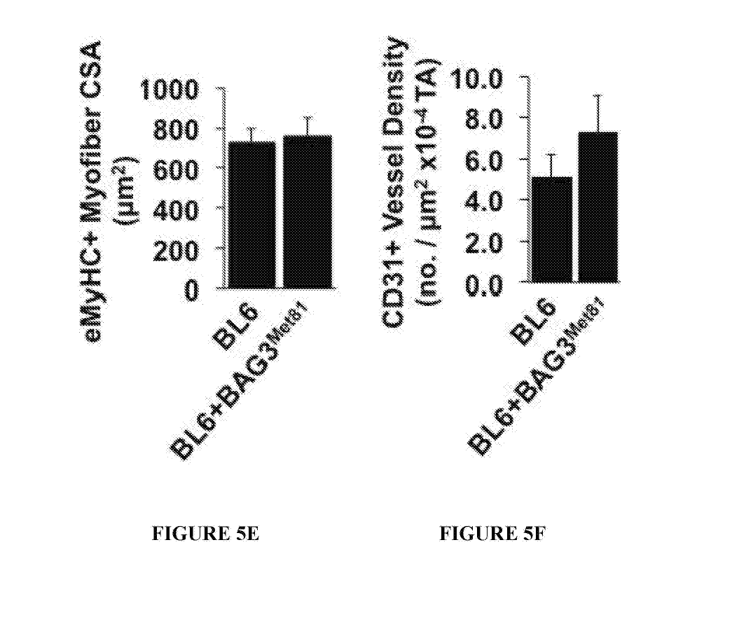

Example 4: BAG3^{Met81} expression in ischemic BL6 mice does not act as a dominant negative inhibitor. Previous genetic studies showed that a single copy of the BAG3^{Ile81} variant, e.g., in first generation offspring of BL6xBALB/c crosses, has a dominant protective effect on ischemic muscle survival. To verify this effect, whether expression of the BALB/c variant, BAG3^{Met81}, in BL6 mice would affect the response to ischemia was investigated. BL6 mice were injected IM with AAV encoding BAG3^{Met81} then subjected to HLI, and no adverse effects on tissue survival were observed. Histological and IF analyses revealed no alterations in muscle morphology, PAX7⁺ cell number, eMyHC expression, regenerating myofiber size, or CD31⁺ vessel density when compared to uninfected BL6 mice 7 days after HLI (FIGS. 5A-5F).

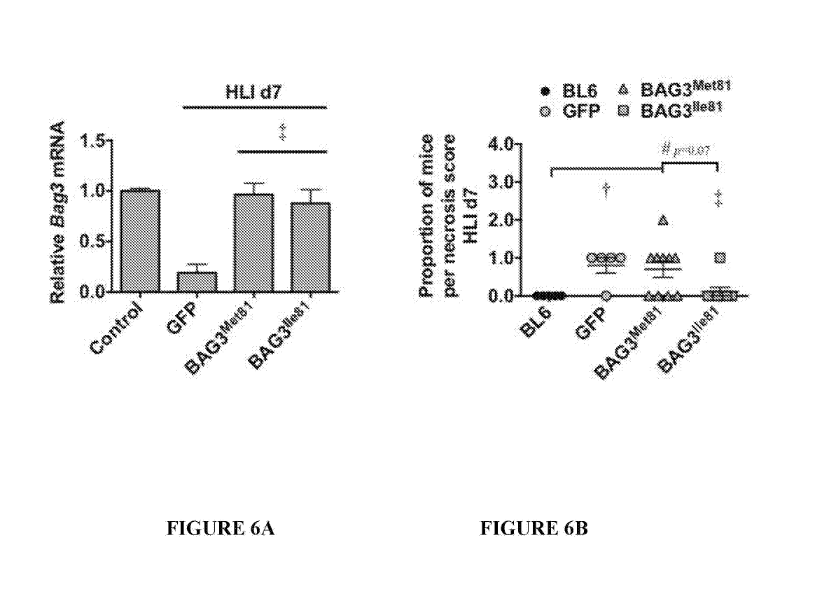

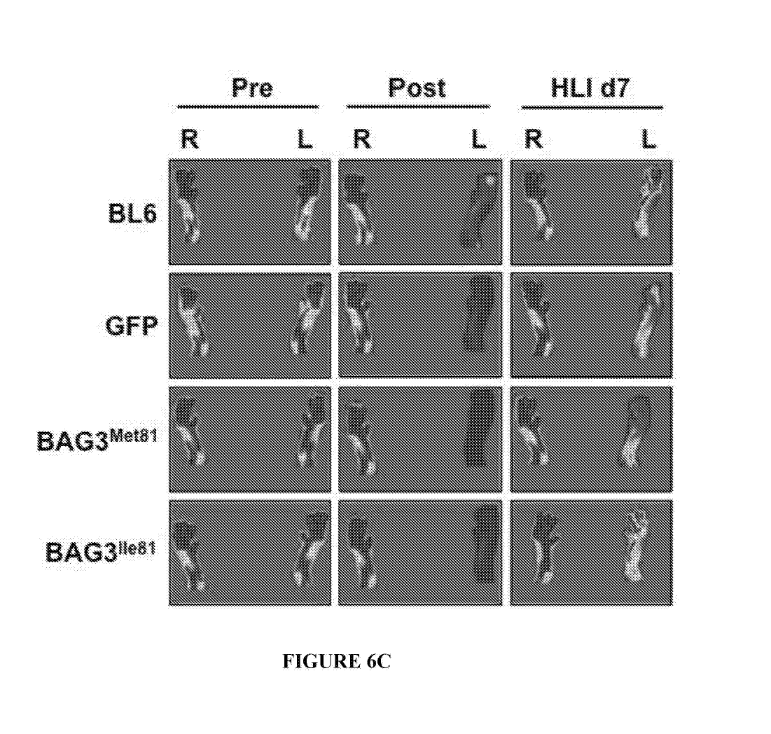

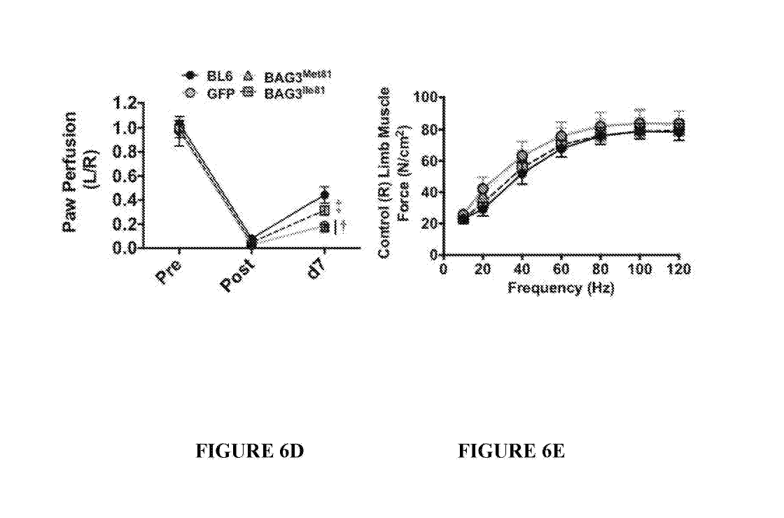

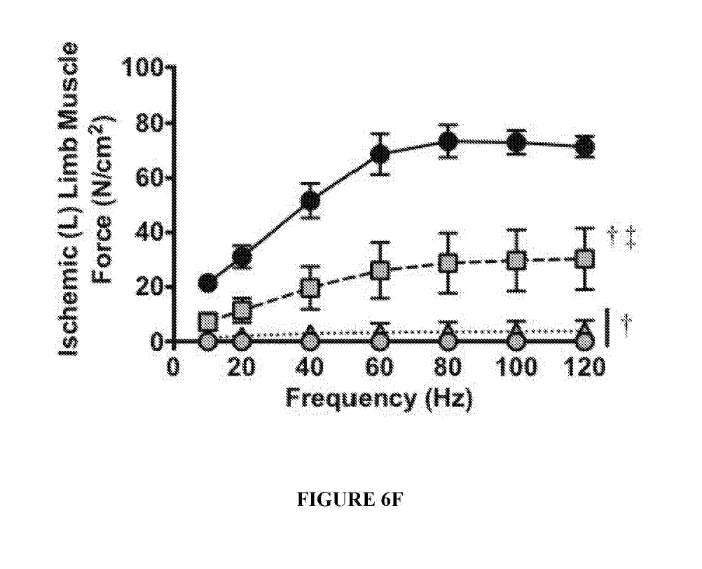

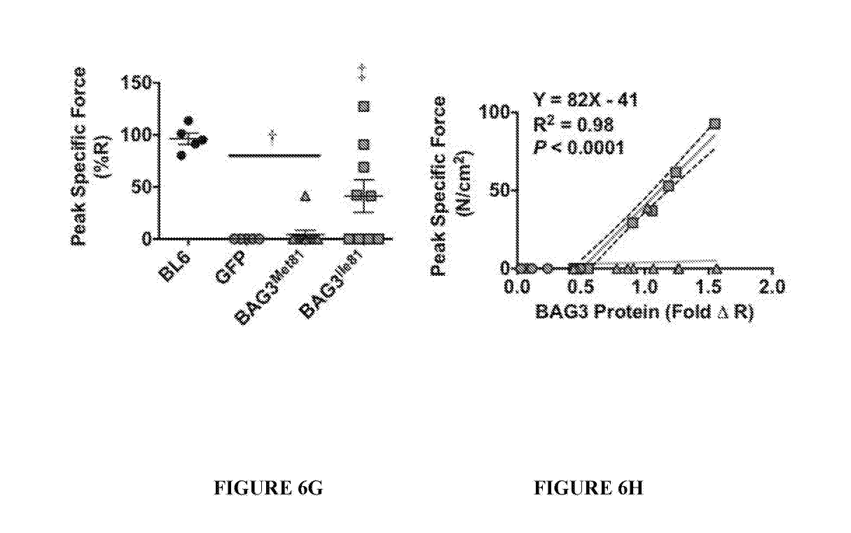

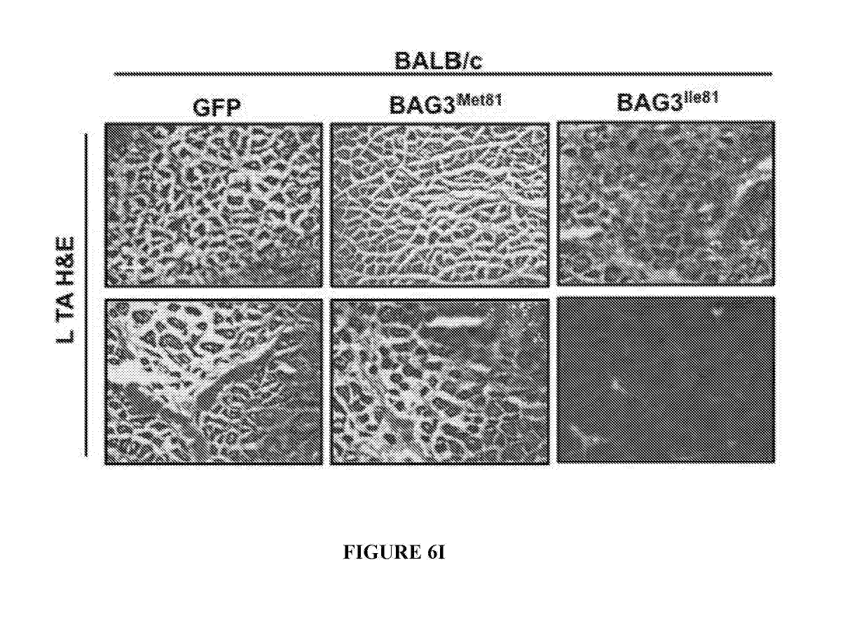

Example 5: Systemic BAG3Ile81 delivery rescues limb blood flow and force production in the ischemic BALB/c limb. Next, investigations were conducted to determine whether BAG3^{Ile81}-mediated muscle regeneration in BALB/c mice resulted in functional muscle improvements (i.e., isometric muscle force production) as measured ex vivo in EDL muscles. Because of the substantial necrosis observed in control BALB/c mice, two modifications were made. First, given the tropism of AAV6 for muscle, AAVs encoding GFP or BAG3 were delivered systemically (by IV injection) to effect expression in all muscle groups of the hind limb, including the extensor digitorum longus (EDL) muscle. Second, the HLI model was refined to limit muscle necrosis by leaving all major collateral vessels intact, which induces less severe limb ischemia. Rescue of ischemic Bag3 mRNA expression was verified by qRT-PCR (FIG. 6A). The modified ischemic injury resulted in mild necrosis only in the GFP- and BAG3^{Met81}-expressing mice (FIG. 6B). Blood flow recovery was significantly less in GFP- and BAG3^{Met81}-treated BALB/c mice, but BAG3^{Ile81} expression returned perfusion recovery to BL6 levels (FIGS. 6C-6D). Force production in non-ischemic control EDL muscle was not different in any treatment group (FIG. 6E). Although force production was significantly impaired in all ischemic BALB/c EDL muscles, BAG3^{Ile81} expression rescued force production across a range of frequencies (FIG. 6F) as well as peak specific force (FIG. 6G) compared to GFP- and BAG3^{Met81}-expressing mice. Correlational analysis revealed a strong positive association between BAG3^{Ile81} expression and limb muscle peak specific force (N/cm²) in BALB/c muscle (FIG. 6H). Muscle histology, qualitatively assessed by H&E, demonstrated intact fascicular arrangements and centralized myofiber nuclei in muscles from BAG3^{Ile81}-expressing mice and degenerative, anucleate myofibers in GFP- and BAG3^{Met81}-expressing ischemic limb muscles (FIG. 6I), similar to the histological changes observed with IM injection of BAG3^{Ile81}.

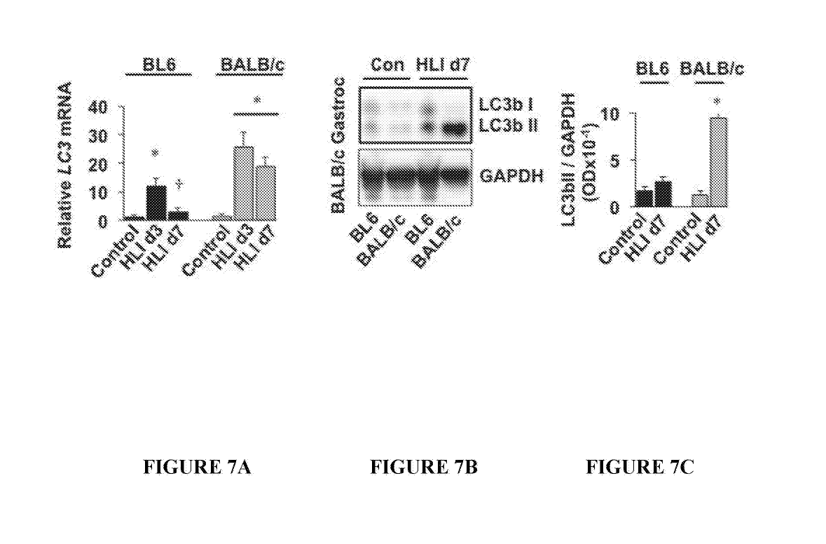

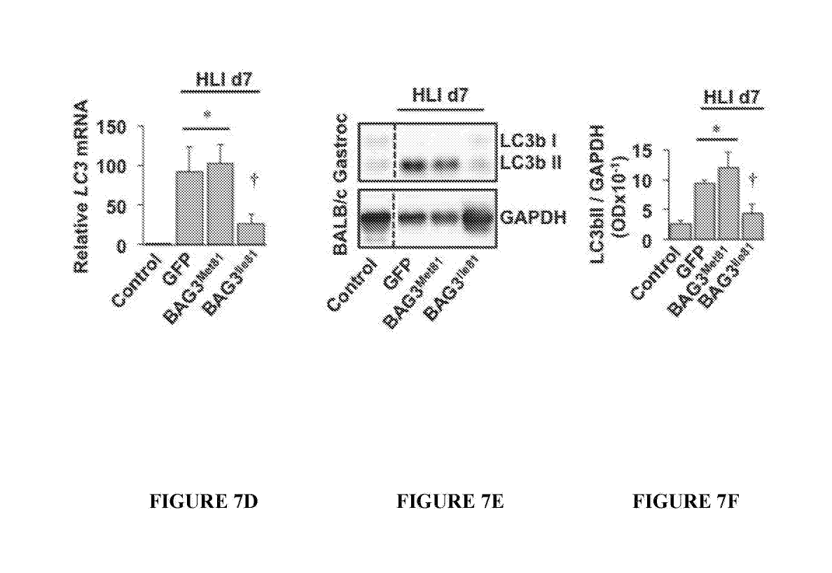

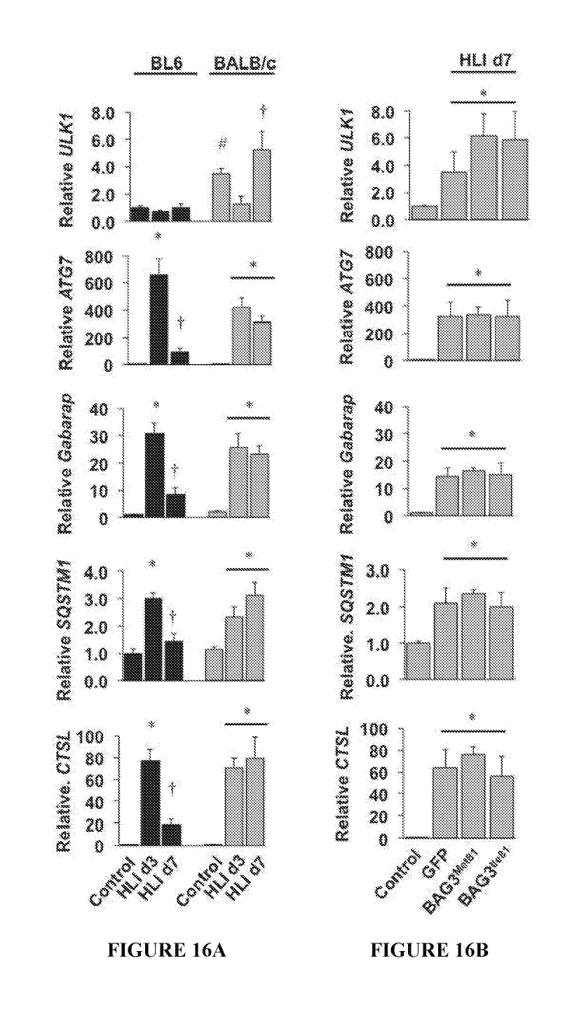

Example 6: Autophagy is differentially regulated in ischemic BALB/c and BL6 muscle and by BAG3 variants. Autophagy is a critical biological process regulating myopathic regeneration in skeletal muscle cells and is believed to partially drive endothelial cell tube formation in vitro, but its role in the differential susceptibility to ischemic myopathy between mouse strains is unknown. To investigate this role, skeletal muscle from the ischemic limbs of BL6 and BALB/c mice was analyzed during the initial week of hindlimb ischemia. Transcriptional analysis demonstrated an increase in LC3 mRNA expression in both BL6 and BALB/c limb muscles 3 days after limb ischemia, which returned to baseline 7 days after HLI only in BL6 mice (FIG. 7A). Other autophagy-related transcripts (ULK1, ATG7, Gabarap, SQSTM1, and CTSL) were similarly induced early after ischemia (HLI d3) in both BL6 and BALB/c limb muscles and decreased 7 days after HLI only in BL6 limb muscles (FIG. 16A). LC3II protein abundance, a product of lipidation, paralleled ischemic mRNA expression at HLI d7 in BL6 and BALB/c mice (FIGS. 7B-7C). In order to understand whether the BAG3 variants differentially affect autophagy in vivo, qRT-PCR was performed on muscle samples from BALB/c mice injected retro-orbitally with AAV-BAG3 variants. BAG3^{Ile81}, but not GFP- or BAG3^{Met81}, reduced LC3 mRNA in BALB/c limb muscle 7 days after HLI (FIG. 7D). However, BAG3^{Ile81} did not alter the expression of other autophagy-related transcripts in vivo (FIG. 16B). LC3II protein abundance 7 days after HLI paralleled the ischemic LC3 mRNA expression in AAV-treated BALB/c mice (FIGS. 7E-7F). Collectively, these data provide direct evidence that autophagy progresses differentially in ischemic BL6 and BALB/c limb muscle and that exogenous expression of BAG3^{Ile81} in BALB/c mice recapitulates the phenotype observed in BL6 mice.

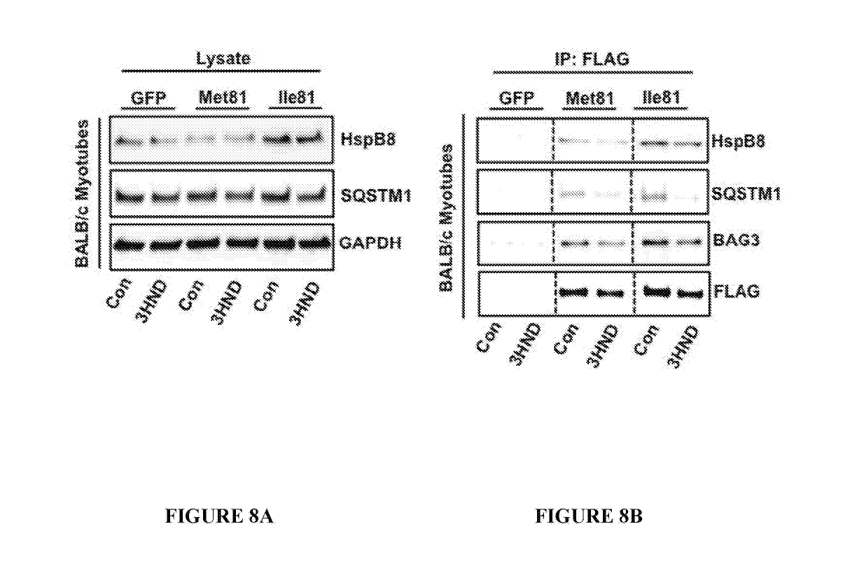

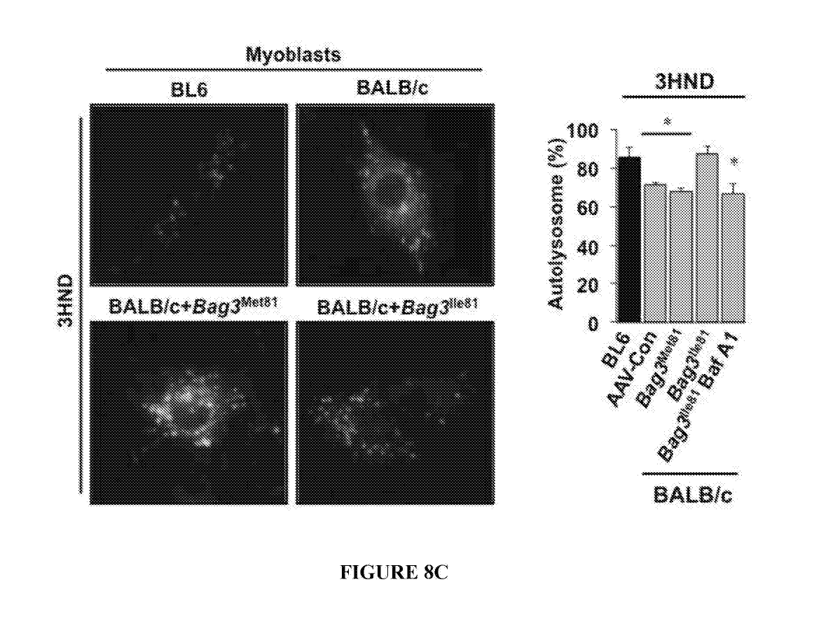

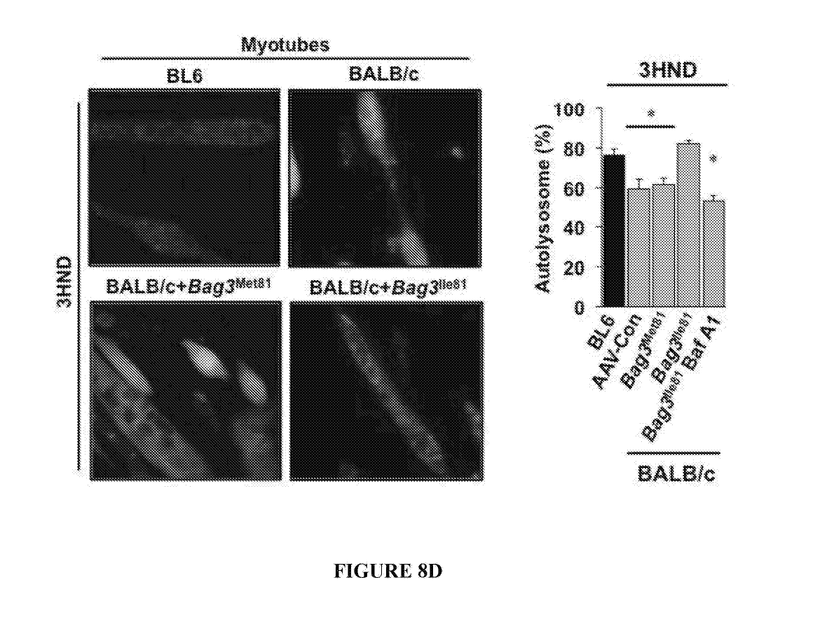

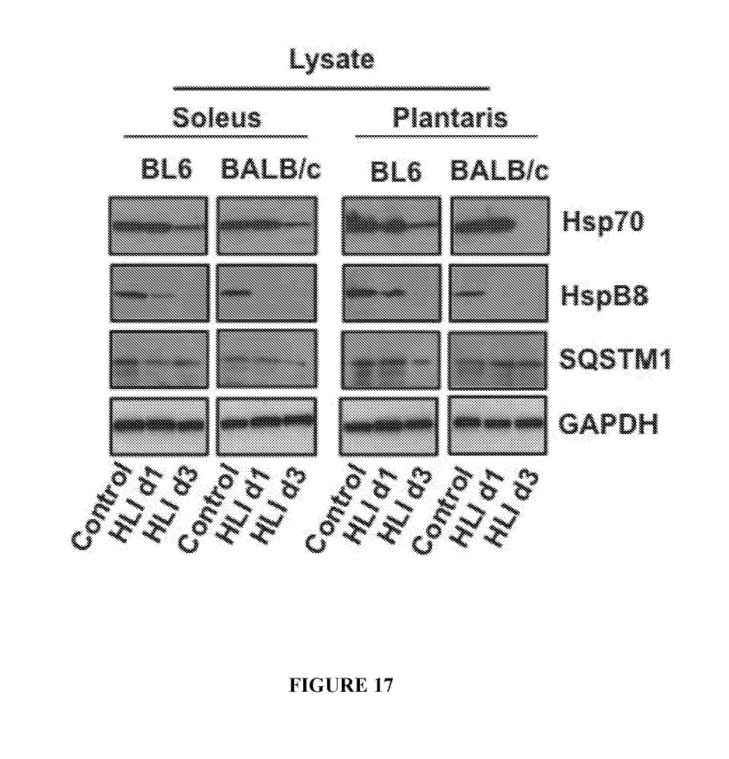

Example 7: BAG3Ile81 differentially binds a small heat shock protein (HspBB) and regulates ischemic muscle cell autophagic flux. The variant amino acid residue 81 flanks the first IPV domain in BAG3⁴⁵, a domain that is known to play a role in directing autophagy. Therefore, the effect of the BAG3 variants on the expression and interaction of proteins linked to autophagic flux was examined. Using limb skeletal muscles early after HLI (days 1 and 3), the protein expression of Hsp70, HspB8, and SQSTM1 (p62) was assessed in BL6 and BALB/c limb muscles. HspB8 demonstrated more persistent expression after ischemia in BL6 muscle compared to BALB/c muscle (FIG. 17). To examine the effects of the BAG3 variants on the interaction with these proteins after ischemia, BALB/c primary muscle cells were injected in vitro with AA Vs encoding GFP, BAG3^{Met81}, and BAG3^{Ile81}, subjected them to experimental ischemia, and blotted whole cell lysates for HspB8 and SQSTM1 (FIG. 8A). The expression of SQSTM1 was not affected by either BAG3 variant, however HspB8 protein was increased by BAG3^{Ile81}. Similarly, immunoprecipitation of FLAG-tagged BAG3 constructs from these lysates (FIG. 8B) revealed greater binding of HspB8 to BAG3^{Ile81}; therefore, the effects of the BAG3 variants on autophagic flux in BALB/c muscle progenitor cells and myotubes were examined. To test this, primary BALB/c myoblasts were co-infected with viruses expressing either BAG3 variant and an RFP-GFP-LC3 reporter. The expression of BAG3^{Ile81}, but not BAG3^{Met81}, rescued autolysosome formation in ischemic BALB/c muscle myoblasts (FIG. 8C) and differentiated myotubes (FIG. 8D) to the level observed in BL6 myoblasts and myotubes, respectively. These results indicate that BAG3^{Ile81} expression in both undifferentiated myoblasts and differentiated myotubes rescues an inherent BALB/c muscle cell impairment in autophagic flux that is paralleled by enhanced binding to HspB8.

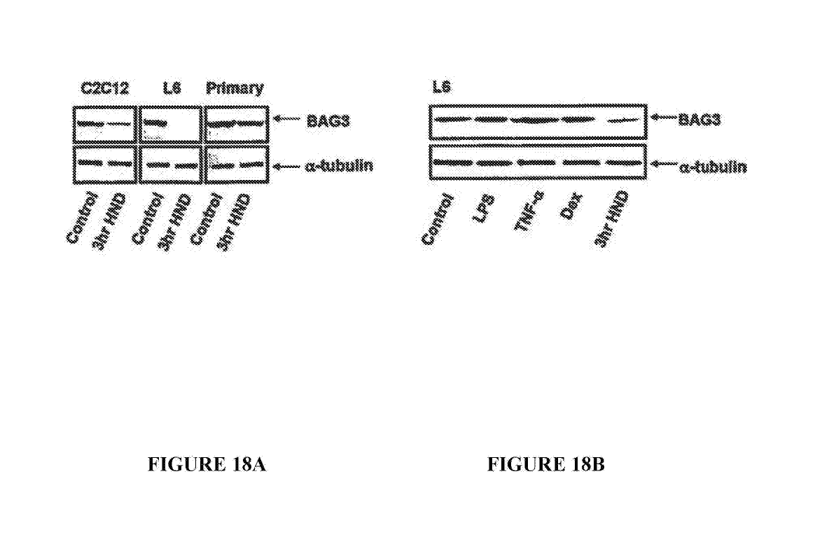

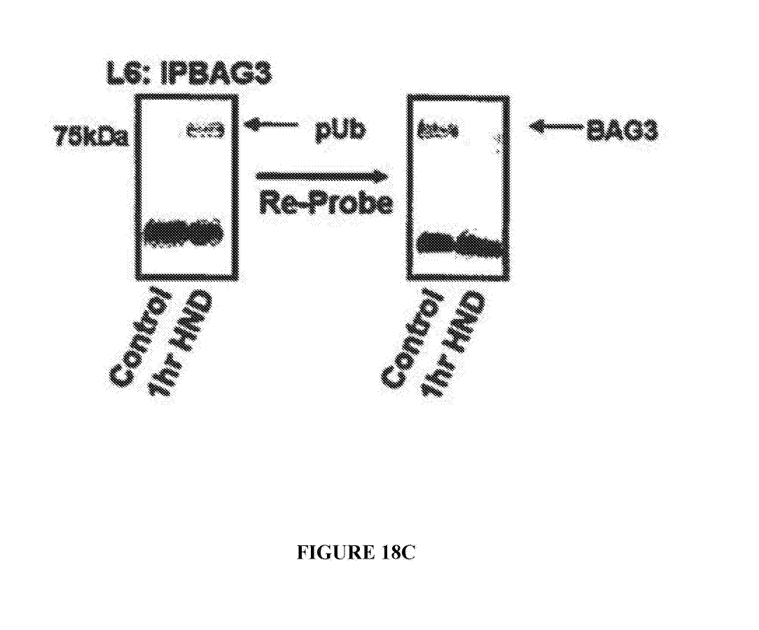

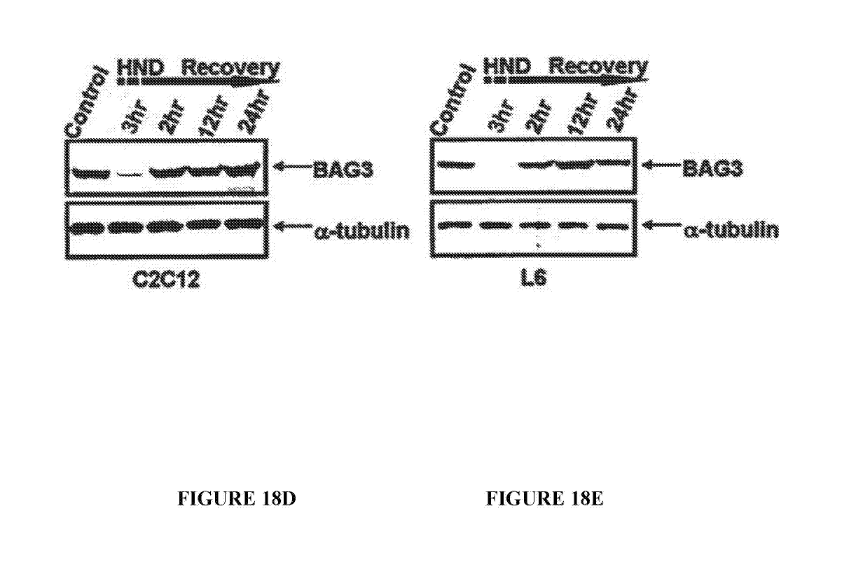

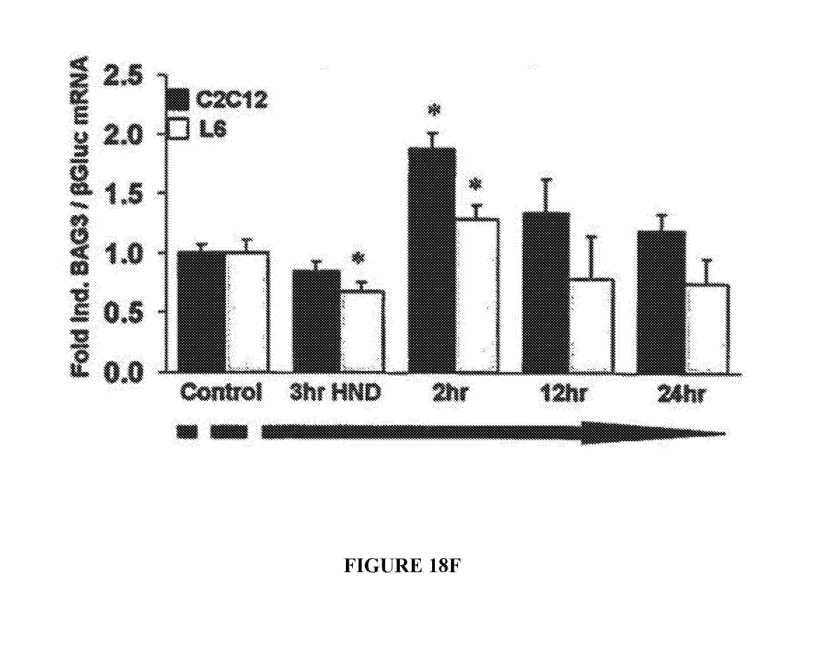

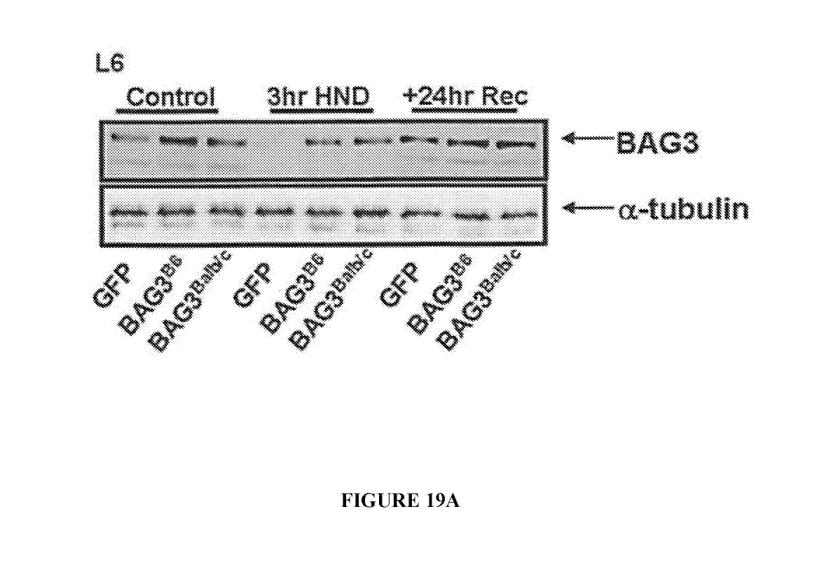

Example 8: Effects of Ischemia/Hypoxia on BAG3. An in vitro model was used to mimic tissue ischemia in skeletal myocytes in which skeletal myoblasts were differentiated to myotubes and then subjected to hypoxia (0% O₂) and complete nutrient deprivation (HND) by changing the medium to Hanks' balanced salt solution for 3 hours. Afterwards, the cells were changed back to differentiation medium and exposed to ambient O₂ concentration (21%). BAG3 protein expression was quickly lost in three different muscle cell lines in this 3-hour window of HND and then recovered within 2 hours (FIGS. 18C, 18D, and 18E). A similar, albeit less dramatic effect was observed on BAG3 mRNA expression (FIG. 18F). Notably, BAG3 expression is not lost after any of a number of other cellular stresses known to cause skeletal muscle damage, including LPS, TNFα, or glucocorticoid treatment (FIG. 18B), suggesting that loss of BAG3 expression is specifically altered by HND. In addition, BAG3 is poly-ubiquitinated (FIG. 18C), suggesting that it is degraded through the 26S proteasome following hypoxia and nutrient deprivation. This effect was noted to various degrees in different cell types, including murine C2C12 cells, rat L6 cells, and, to a lesser extent, primary murine myocytes (FIGS. 18A-18F). Although the role of BAG3 loss in the setting of hypoxia is not entirely clear, data from an in vivo hind limb ischemia (HLI) model demonstrate a similar loss of BAG3 protein and RNA expression. Nonetheless, these in vitro findings raise the possibility that re-expression of BAG3, particularly the protective Ile81 genotype, might have a beneficial effect on skeletal muscle biology during hypoxia/ischemia.

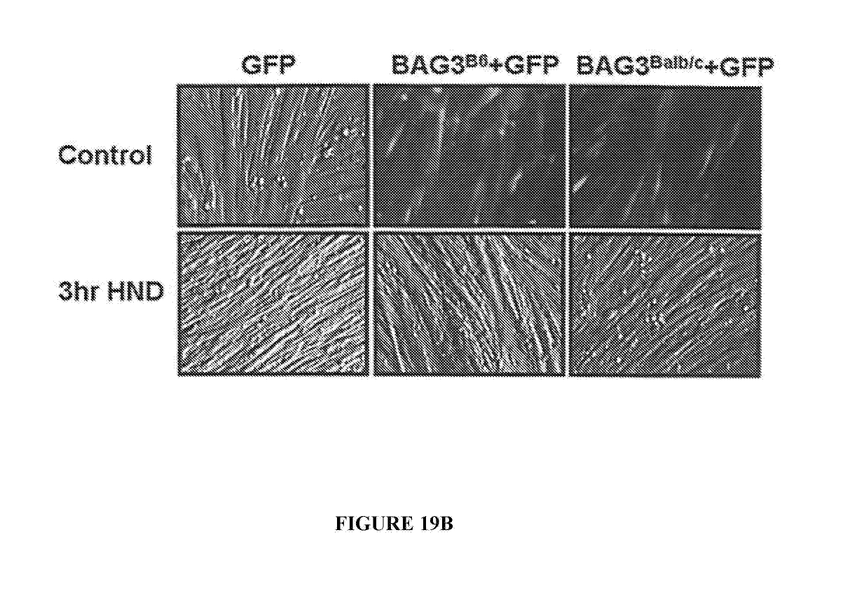

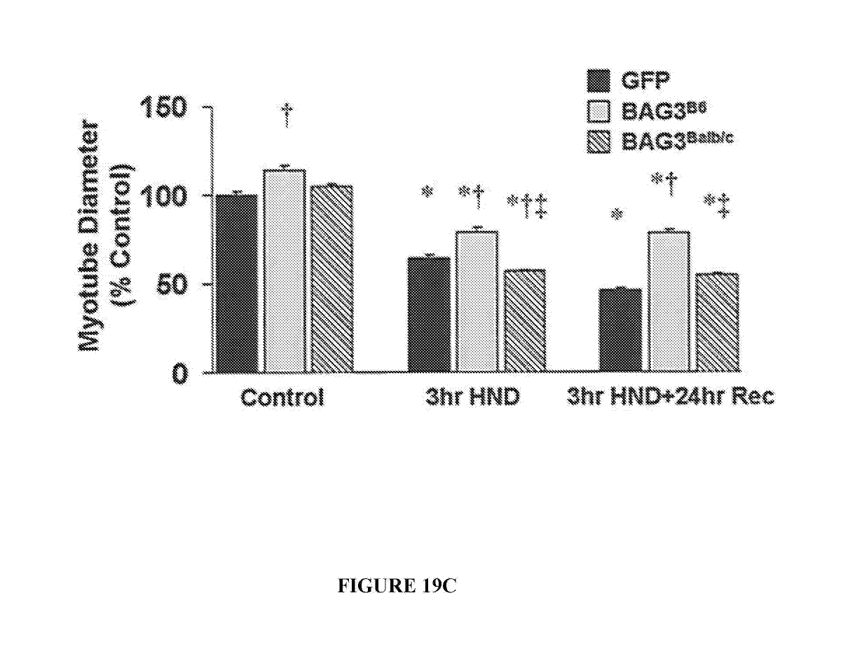

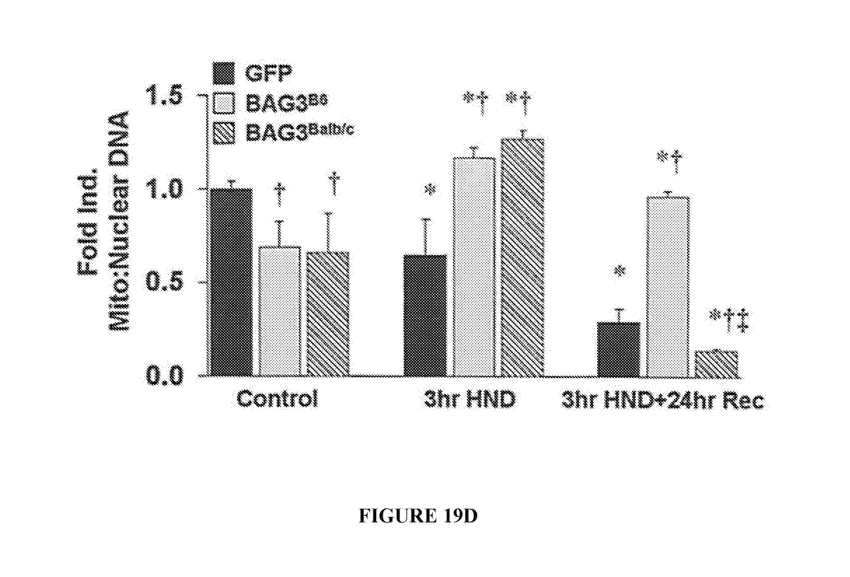

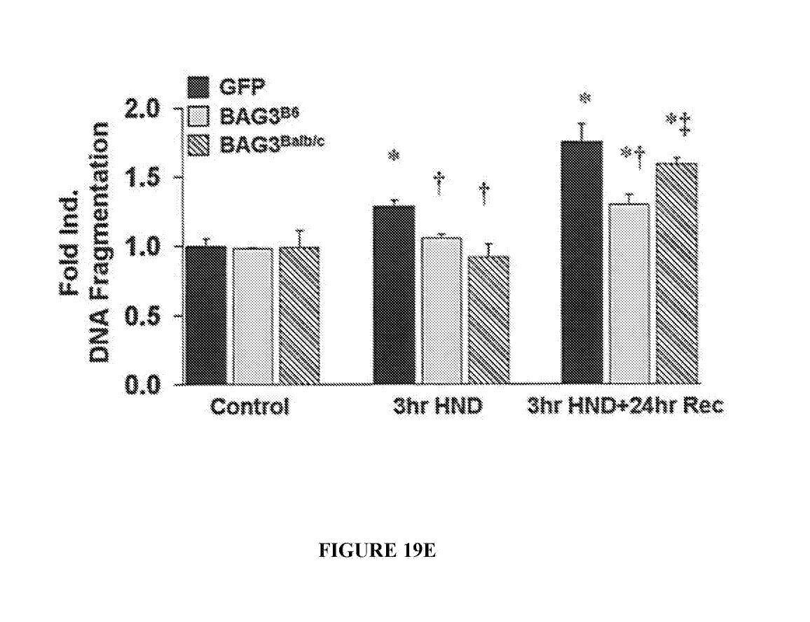

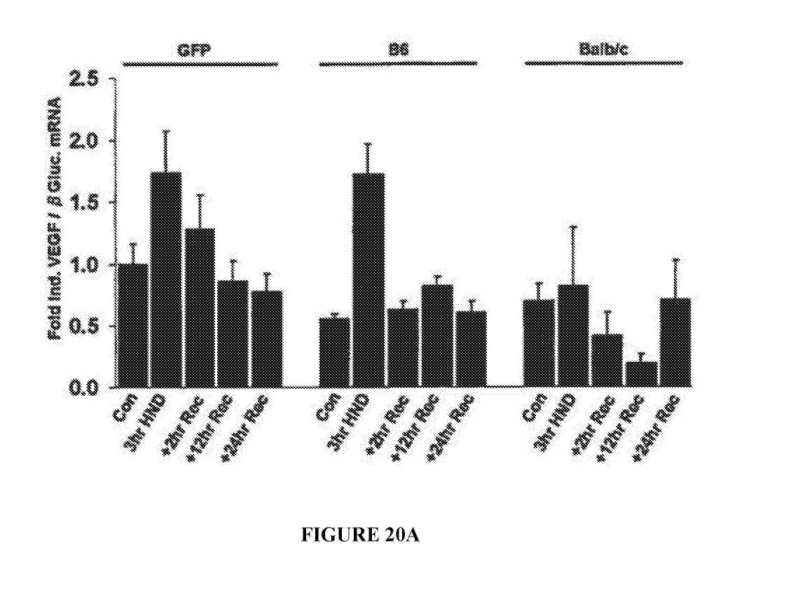

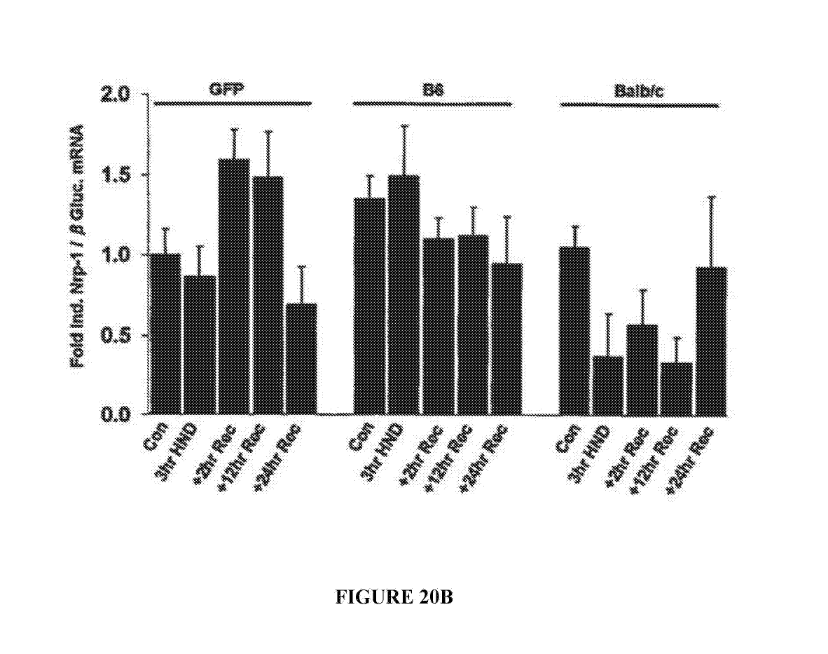

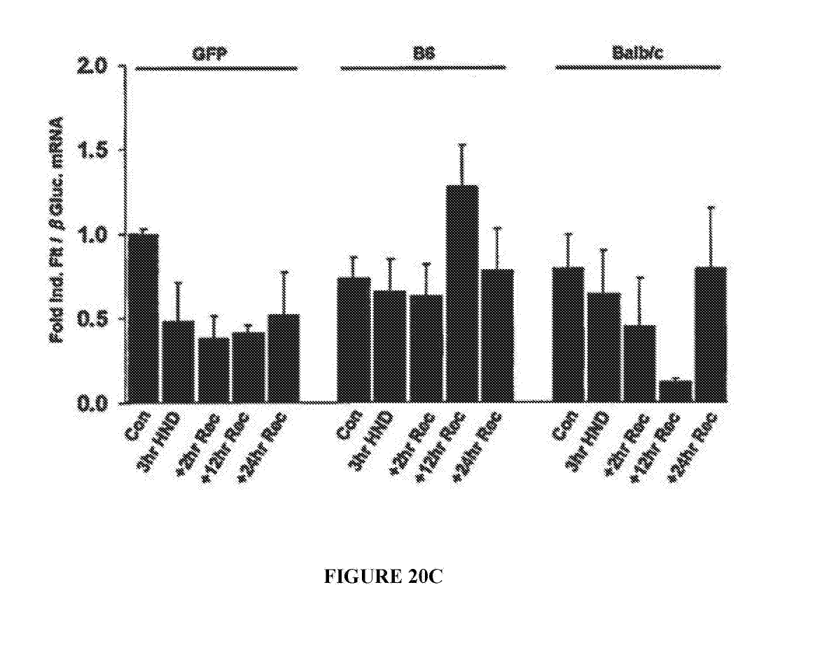

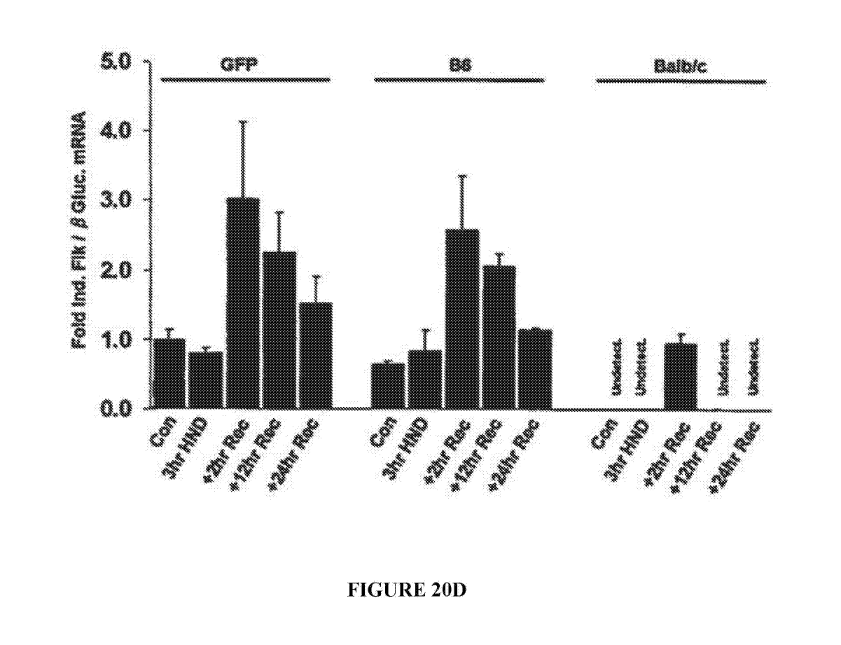

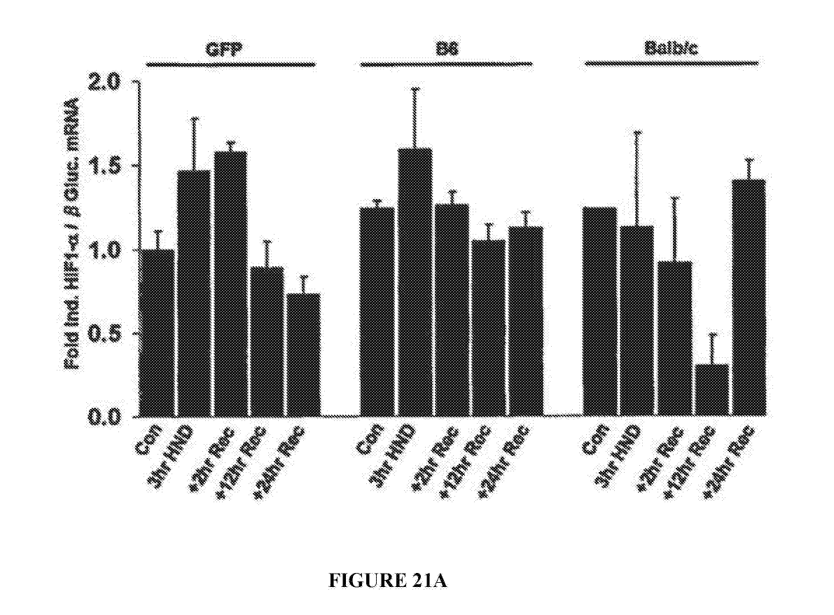

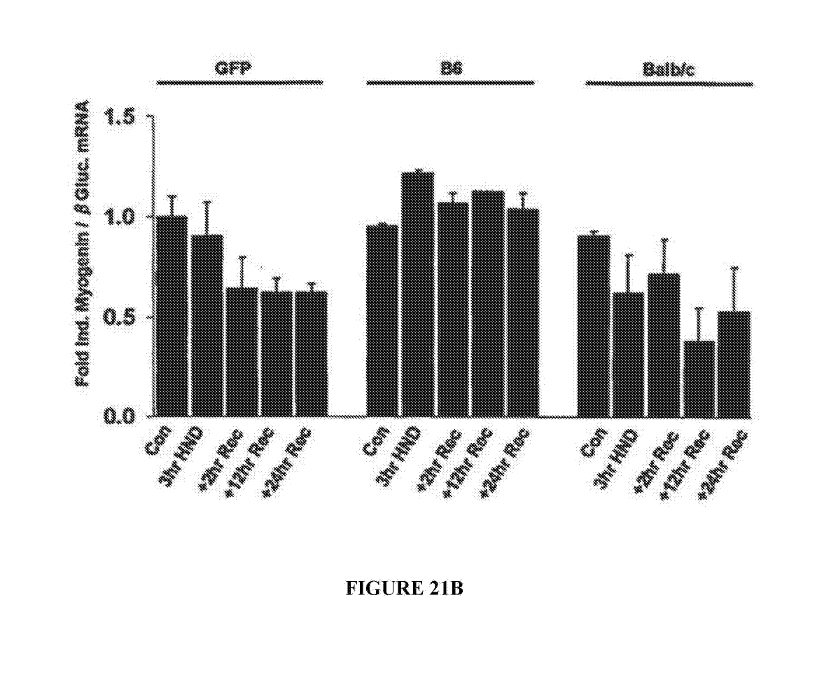

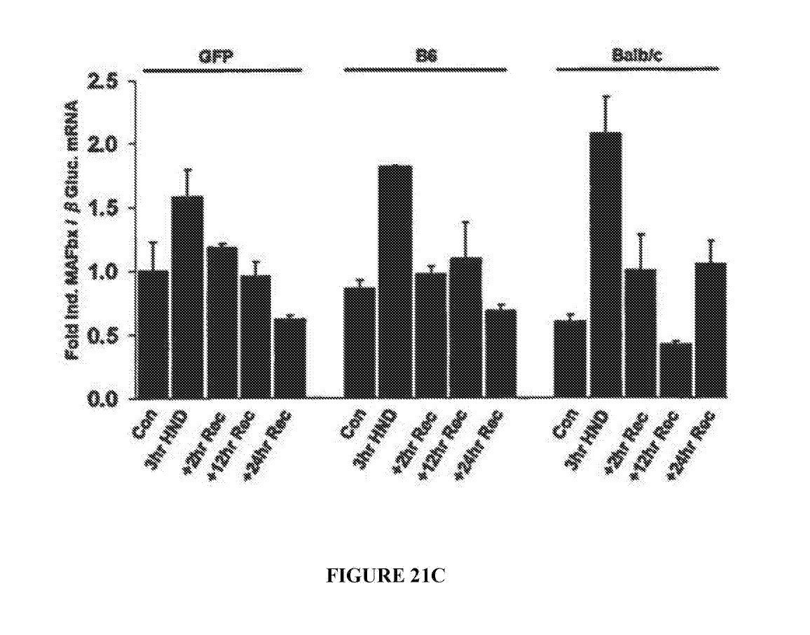

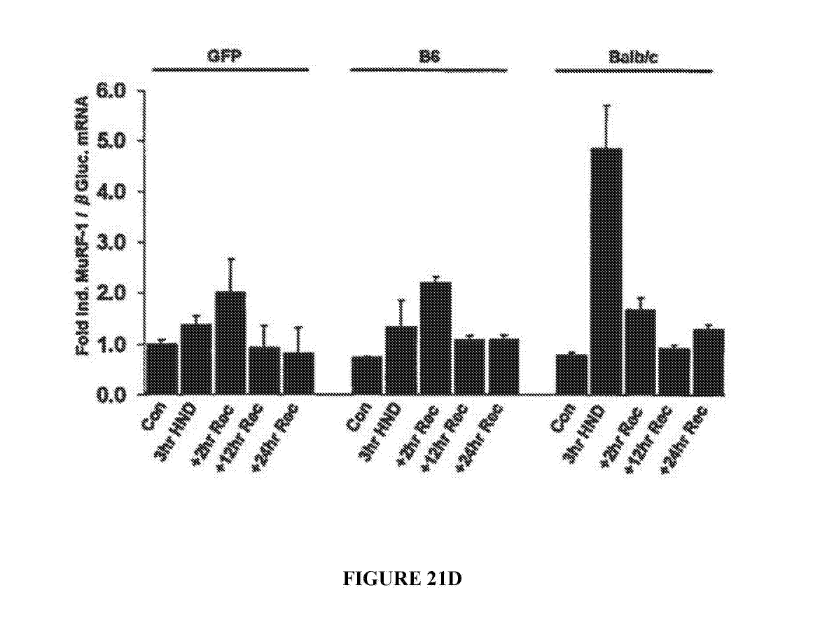

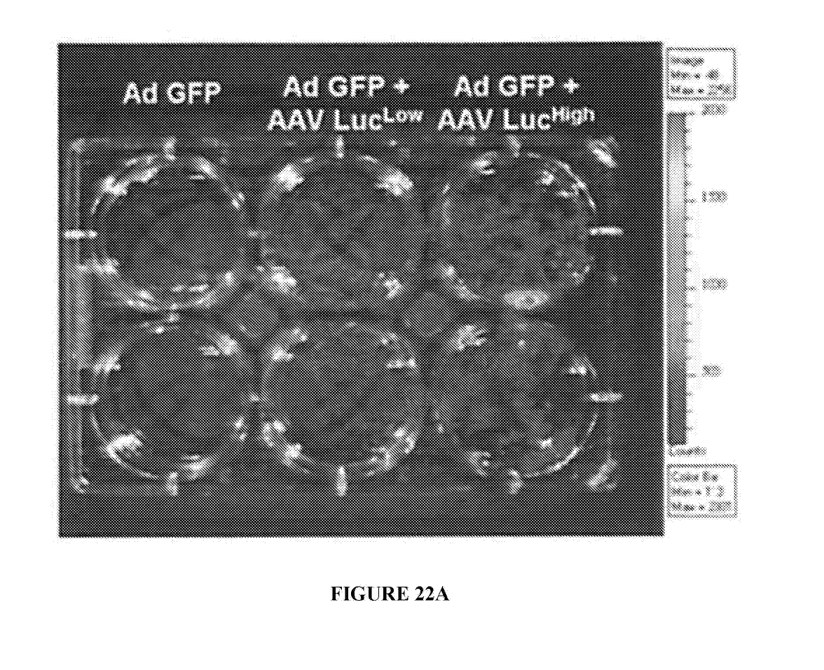

To test this possibility in vitro, rat L6 myocytes were used, since the endogenous rat BAG3 contains a distinct residue at position 81 (alanine). L6 cells were transfected with plasmids encoding GFP as a control or BAG3 from the two different inbred mouse strains known to respond differentially to limb ischemia, C57BL/6 (86), which expresses BAG3^{Ile81}, or BALB/c, which expresses BAG3^{Met81}. Transient transfection with plasmid DNA encoding either genotype of BAG3 rescued the HND-induced loss of endogenous BAG3 (FIGS. 19A-19B), indicating that the genotype, per se, was not responsible for BAG3 degradation. However, BAG3^{Ile81} had a beneficial effect on L6 myocytes during the recovery from HND. In cells expressing BAG3^{Ile81}, but not BAG3^{Met81} or GFP, recovery of myotube diameter (i.e., atrophy) (FIGS. 19B-19C), mitochondrial number (FIG. 19D), and DNA fragmentation (apoptosis) (FIG. 19E) were all significantly improved. Similarly, expression of BAG3^{Ile81} had beneficial effects on myocyte expression of the angiogenic factor vascular endothelial growth factor (VEGF) and its receptors, neuropilin (Nrp)-1, VEGFR-1 (Flt), and VEGFR-2 (Flk) (FIG. 20) as well as the myogenic regulatory factor myogenin and the muscle-specific ubiquitin ligase MuRF-1 (FIG. 21).

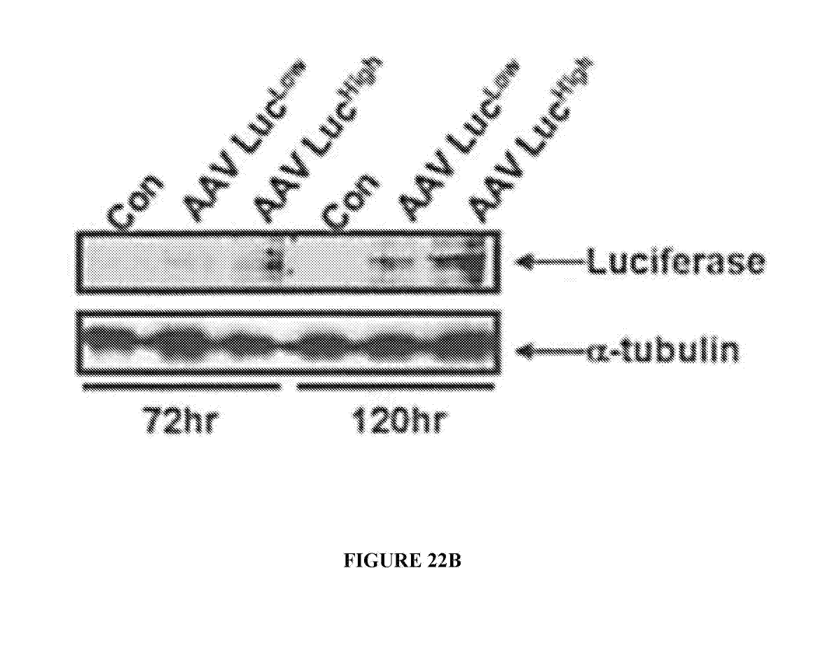

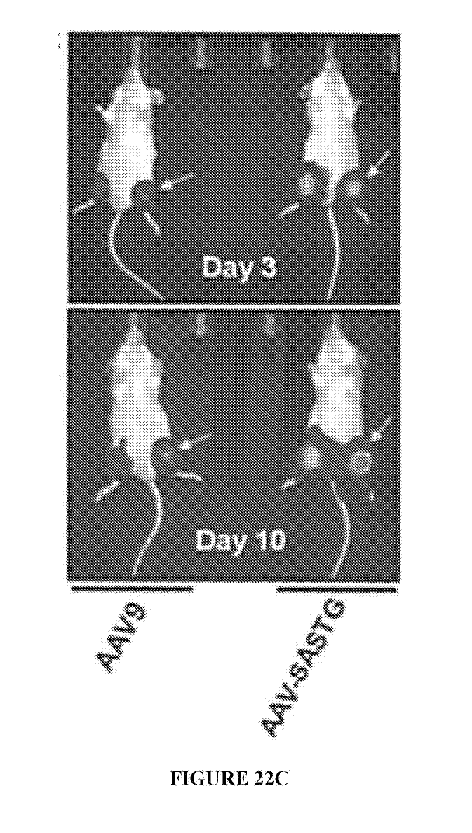

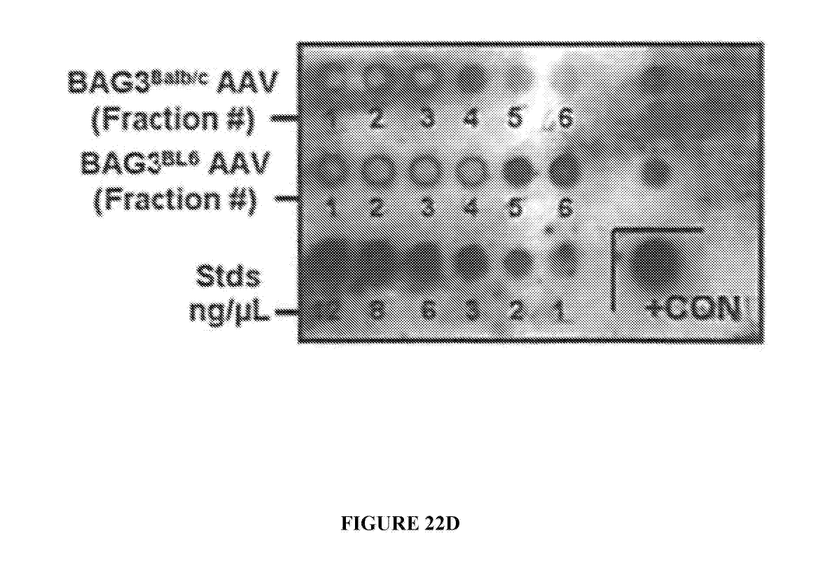

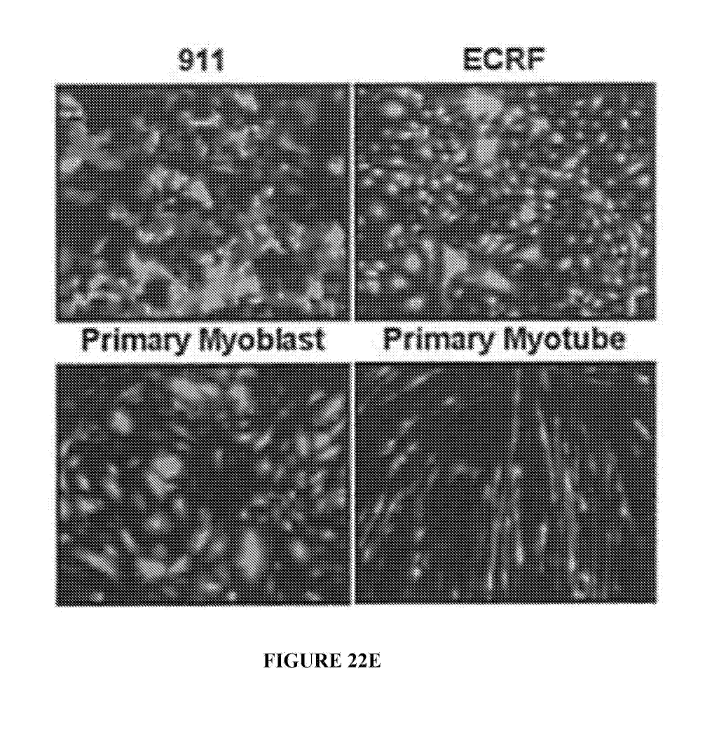

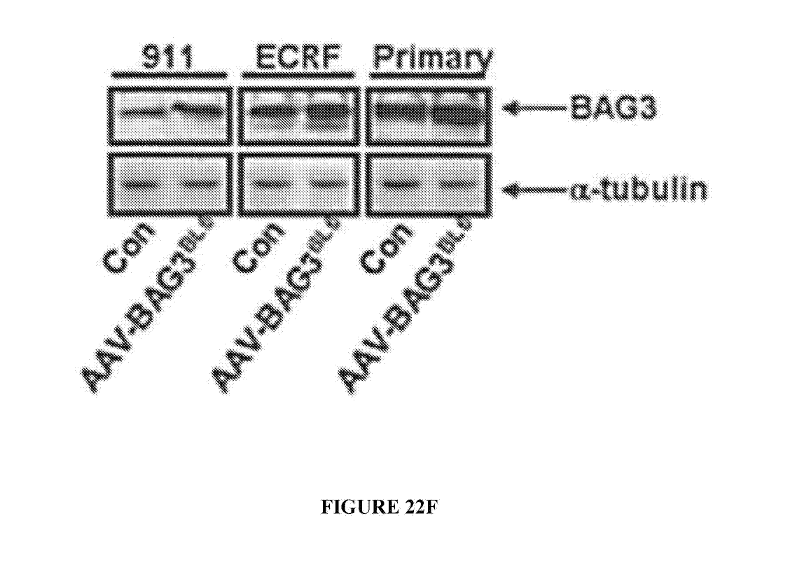

Example 9: In vivo administration and effects of BAG3Ile81. The in vitro data above suggested that delivery of BAG3^{Ile81} might have a beneficial effect on ischemic muscle in vivo. To test this possibility, a recombinant adeno-associated virus (AAV) vector encoding BAG3^{Ile81} was developed to test its effects in ischemic skeletal muscle, although one of ordinary skill in the art envision based on the present disclosure other strategies being employed, as described above, including recombinant adenovirus or naked plasmid DNA. Recombinant AAV would be expected to have certain advantages over these other strategies for skeletal muscle-specific expression, including longer duration of expression and reduced immunogenicity compared to adenovirus. Moreover, the trophism of AAVs can be modified to target specific cell types, including skeletal myocytes, using vectors with specific capsids. For example, AAV9 has been used frequently to target skeletal muscle, but a modified capsid vector termed SASTG results in higher transgene expression in muscle tissue, possibly through improved targeting of endothelial and other cell types (FIG. 22C). However, it remains unclear whether BAG3 delivery would also have beneficial effects on the endothelium in the setting of ischemia. Infection of primary myoblasts as well as other cell types (911 epithelial cells or ECRF endothelial cells) with AAV-BAG3^{Ile81} demonstrates efficient expression of BAG3 (FIG. 18F).

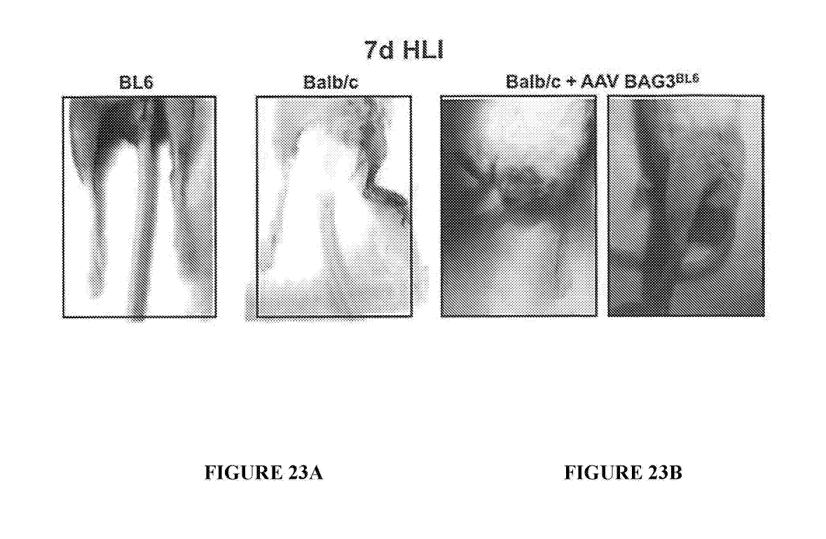

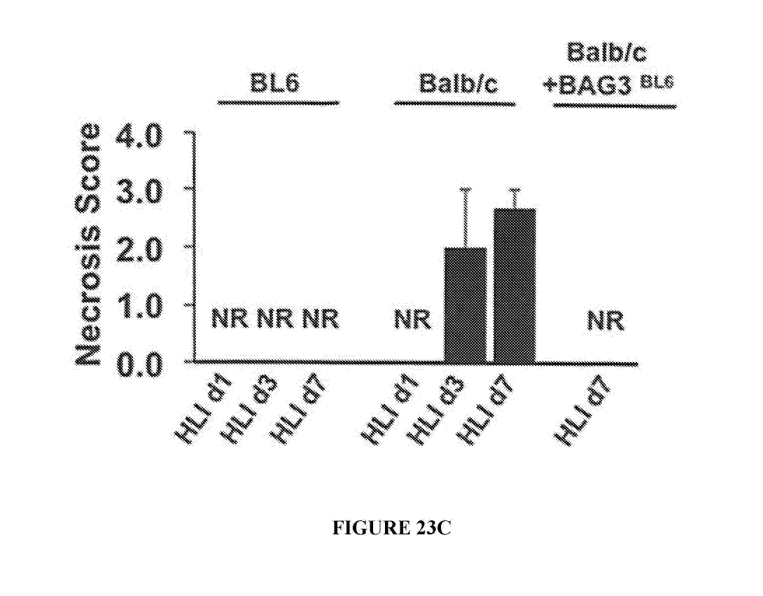

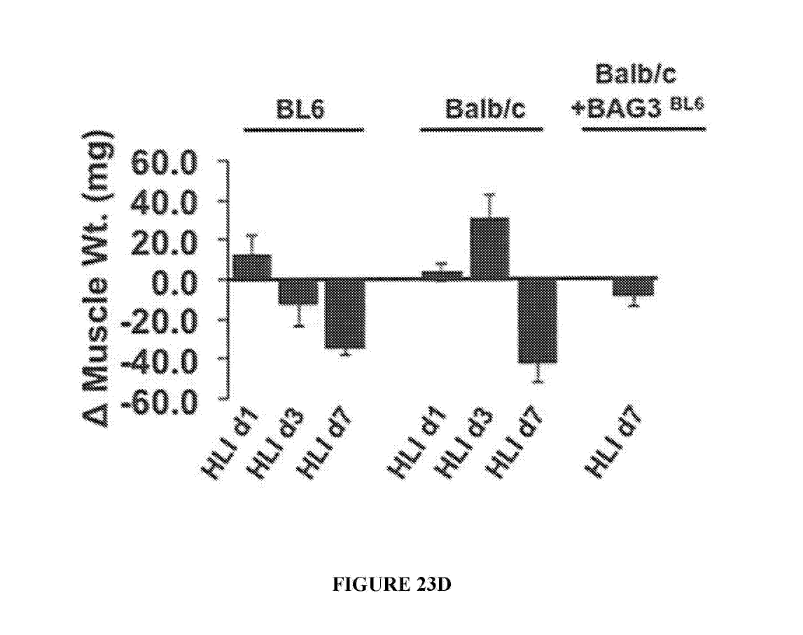

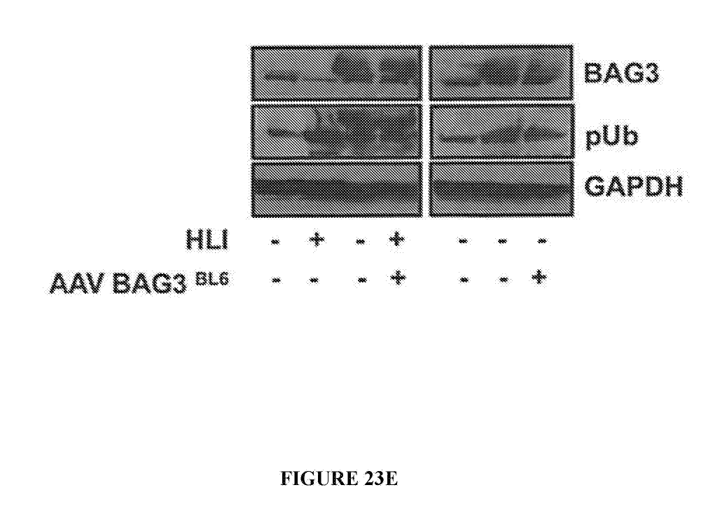

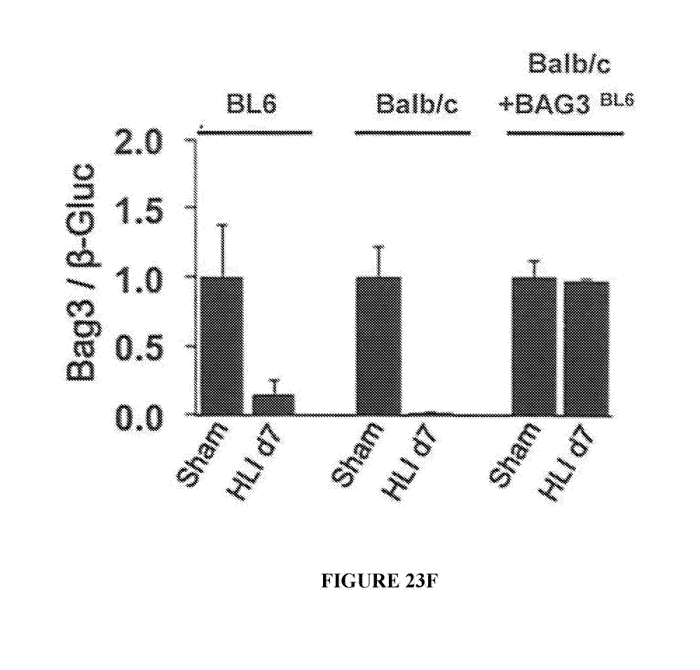

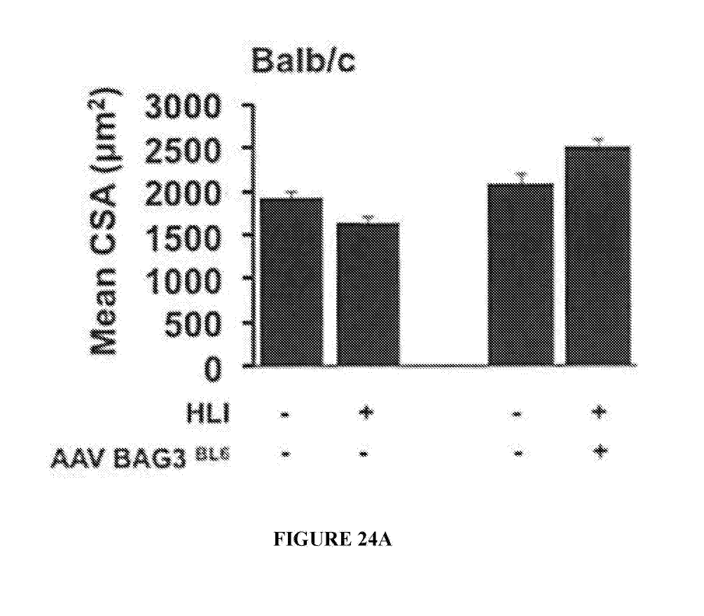

To test effects of BAG3^{Ile81} on ischemic skeletal muscle in vivo, BALB/c mice were subjected to hind limb ischemia (HLI) by ligation and excision of the left femoral artery between the inguinal ligament and popliteal artery, as described previously. As noted above, this degree of HLI typically results in varying degrees of limb necrosis in BALB/c mice (FIG. 23A, right), in contrast to C57BL/6 mice (FIG. 23A, left), which seldom develop limb necrosis. BALB/c mice underwent 3 separate injections of AAV-BAG3^{Ile81} into the tibialis anterior muscle and both heads of the gastrocnemius muscle (anterior and posterior lower limb muscles, respectively). Seven days later (to allow time for transgene expression), mice were subjected to femoral artery ligation. Animals were either sacrificed prior to surgery to allow analysis of BAG3^{Ile81} expression and effects on baseline muscle tissue morphology, architecture, and capillary density 7 days after infection, or they were subjected to HLI for 7 days and then sacrificed for analysis of BAG3 expression and tissue morphology (i.e., 14 days after BAG3^{Ile81} delivery). Unlike control BALB/c mice, limbs of AAV-BAG3^{Ile81}-treated mice looked completely normal (FIG. 23B) and failed to develop necrosis by a semi-quantitative necrosis score (FIGS. 23B-23C). Limb perfusion appeared normal, and muscle weight was rescued (i.e., ischemia-induced atrophy was inhibited), even compared to that in wild-type C57BL/6 mice (FIG. 23D). AAV-BAG3^{Ile81} delivery restored BAG3 protein (FIG. 23E) and mRNA expression (FIG. 23F) to normal, pre-ischemic levels. Thus, BAG3 was not over-expressed in this model relative to baseline expression.

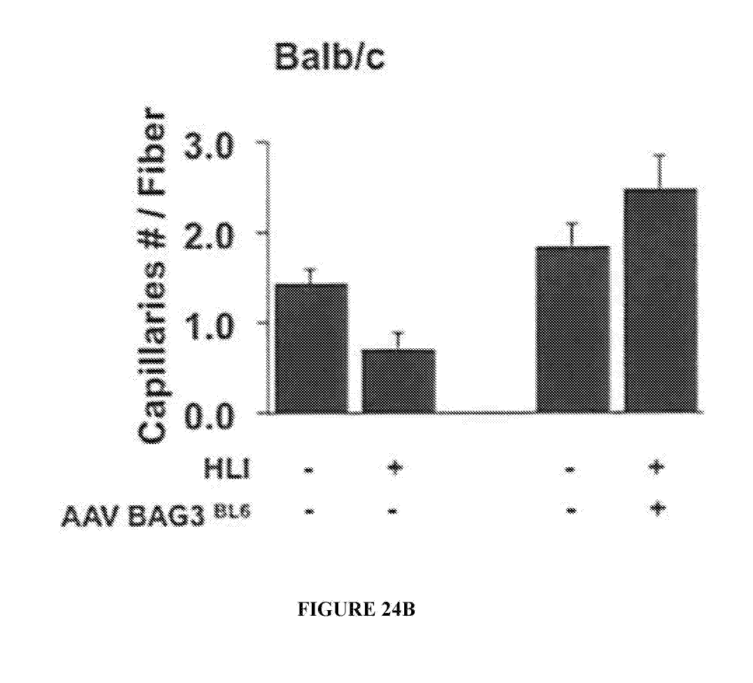

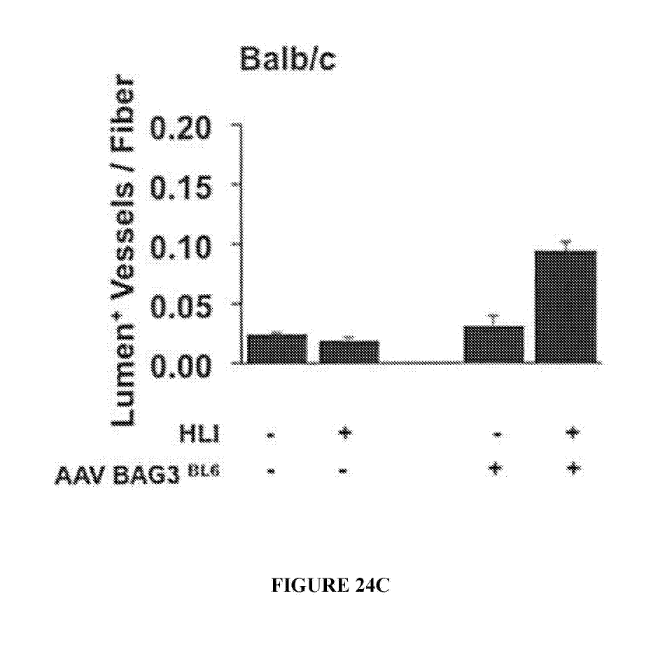

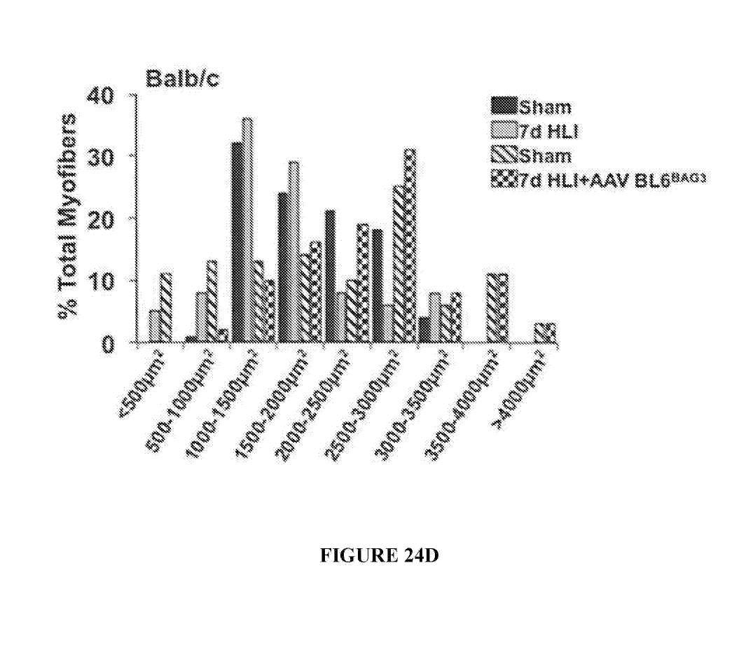

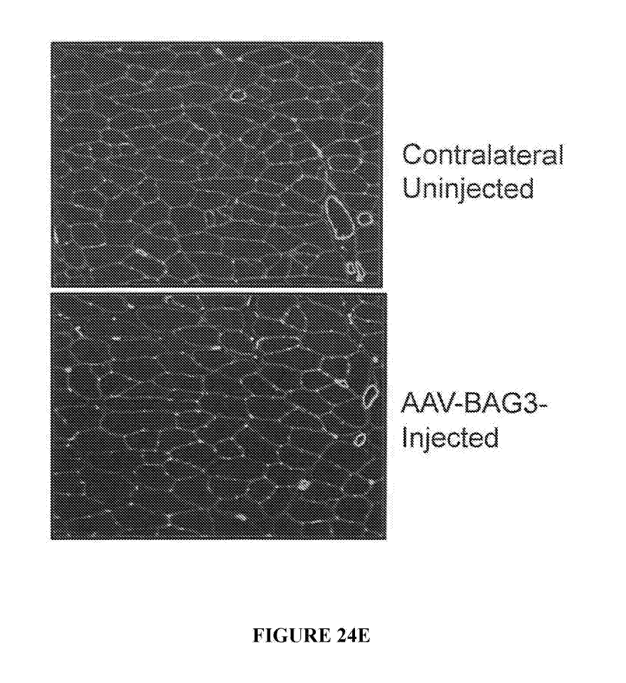

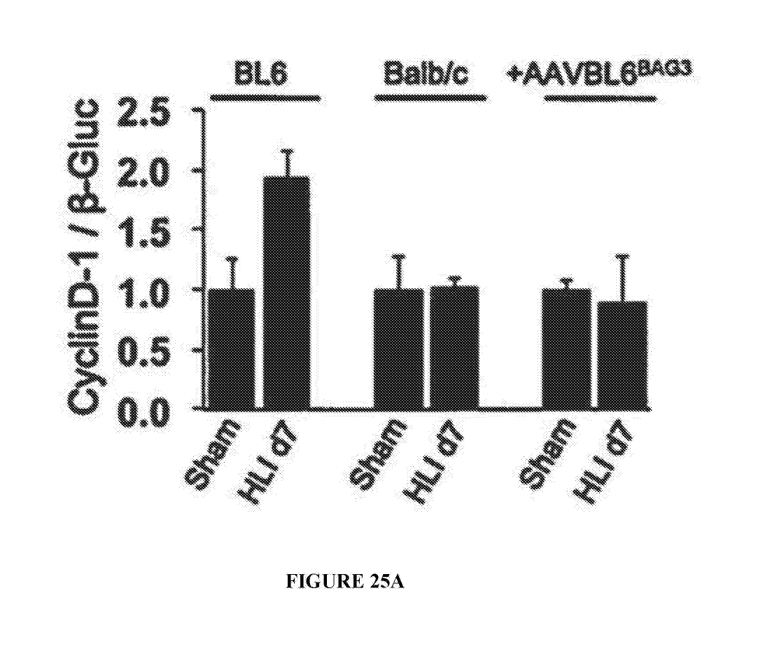

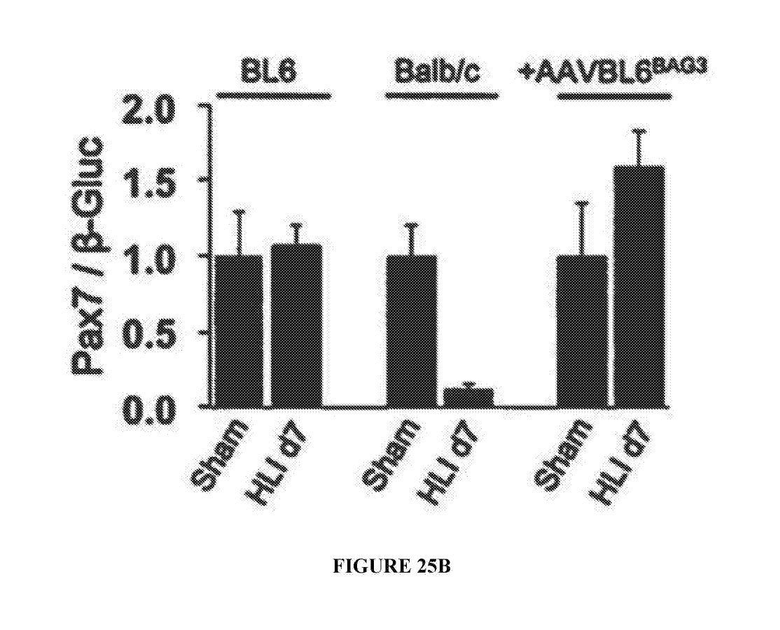

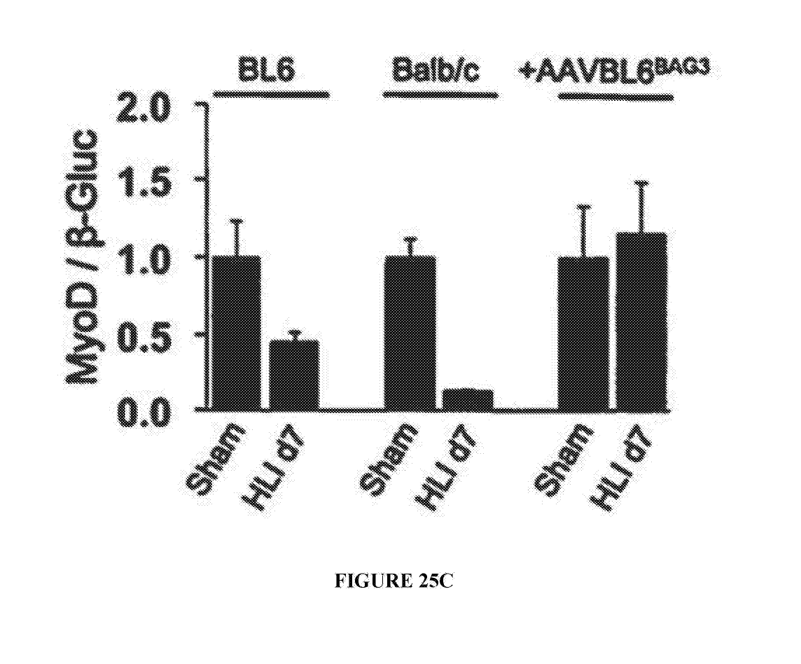

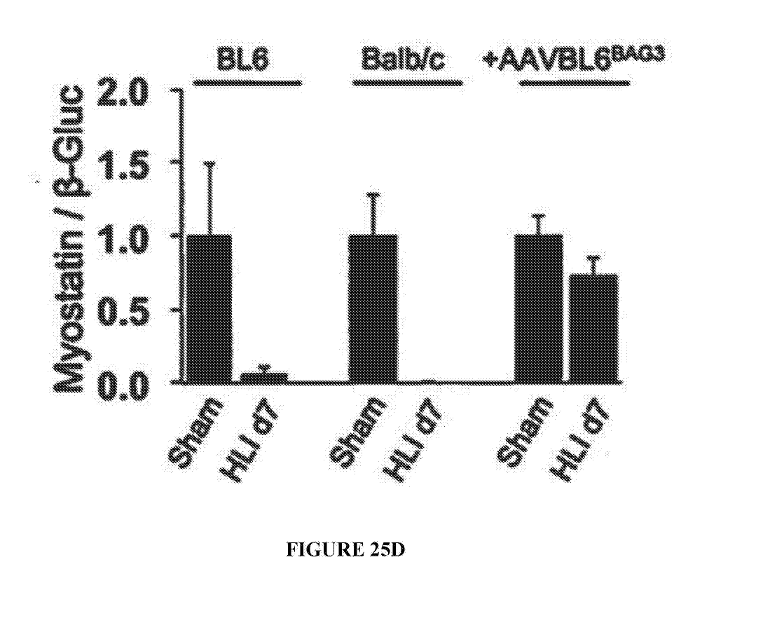

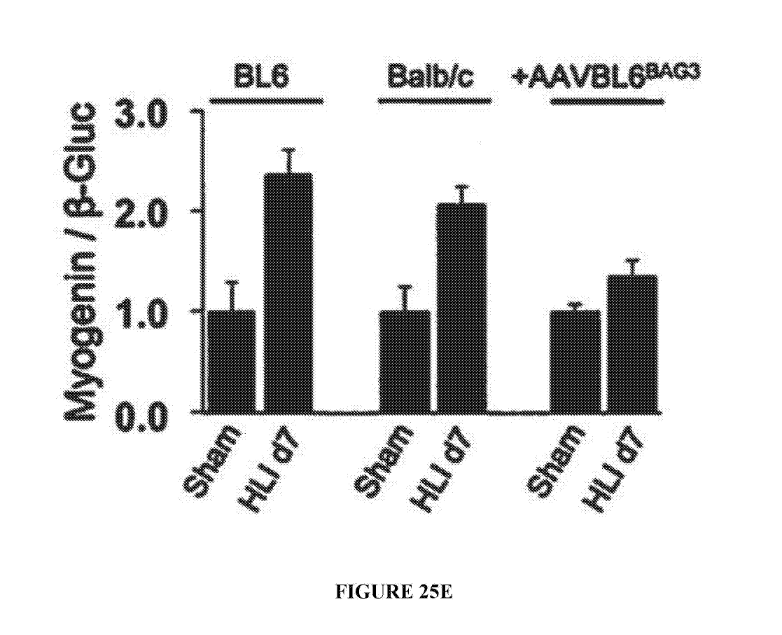

Strikingly, in addition to protecting the ischemic limbs from necrosis, the tissue demonstrated dramatic improvements in a number of other parameters adversely affected by ischemia (FIG. 24E), including muscle fiber cross-sectional area (FIG. 24A), capillary density (capillaries per muscle fiber) (FIG. 24B), and vascular luminal diameter (FIG. 24C). Quantitation of fiber diameters revealed many more large fibers, indicating either protection from atrophy or overt hypertrophy in response to the BAG3 transgene (FIG. 24D). Moreover, whereas both wild-type C57BL/6 and BALB/c mice display a variety of changes in gene expression following HLI, BALB/c mice injected with AAV-BAG3^{Ile81} appeared to be completely protected from detrimental effects of ischemia (FIG. 25). For example, loss of Pax7 expression (a marker of muscle stem cells) in BALB/c mice is not only reversed with AAV-BAG3^{Ile81}, but it is markedly increased (FIG. 25B). Similarly, the loss of myoD and myostatin and the increase in myogenin, which are important for promoting new myocyte growth after HLI and are observed to varying degrees in both strains of mice, are completely inhibited by BAG3^{Ile81} (FIGS. 25C-25E). These findings suggest that BAG3^{Ile81} expression protects skeletal muscle from the detrimental effects of ischemia, likely through direct effects on myocytes' ability to resist hypoxia, and at least in part by enhancing vascular growth, through angiogenesis and/or arteriogenesis.

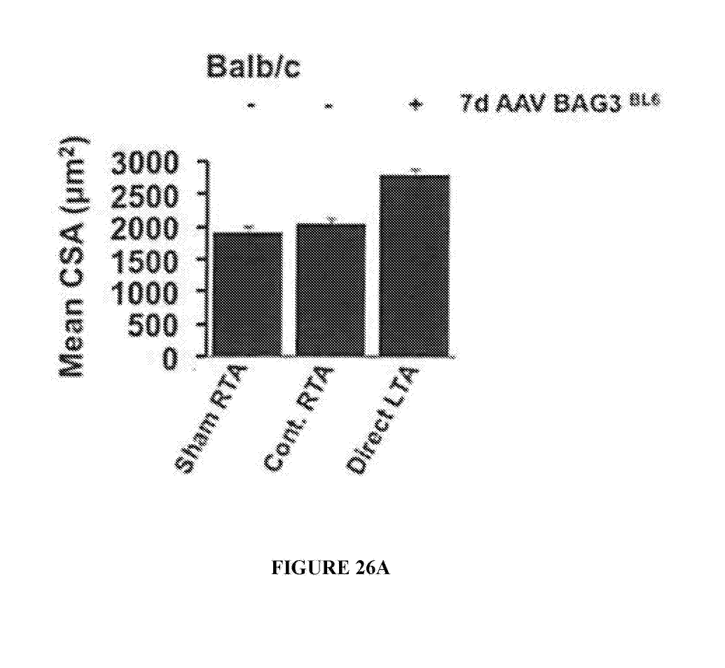

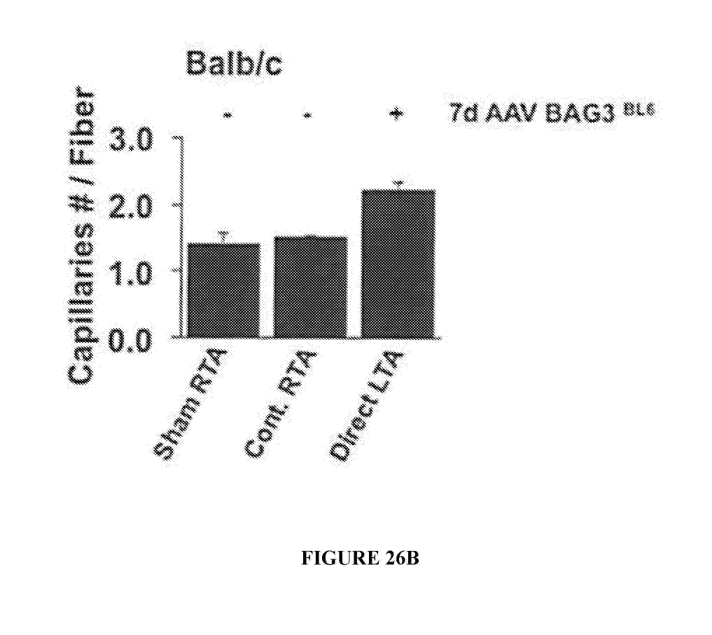

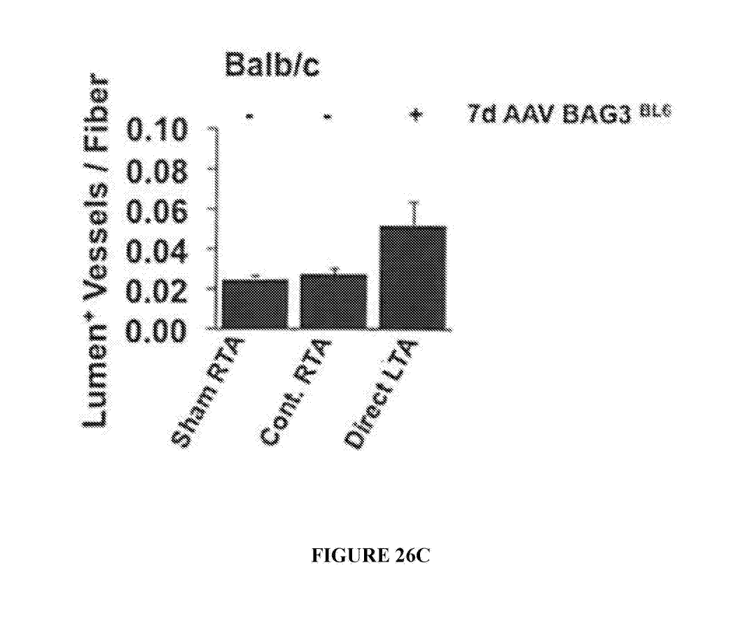

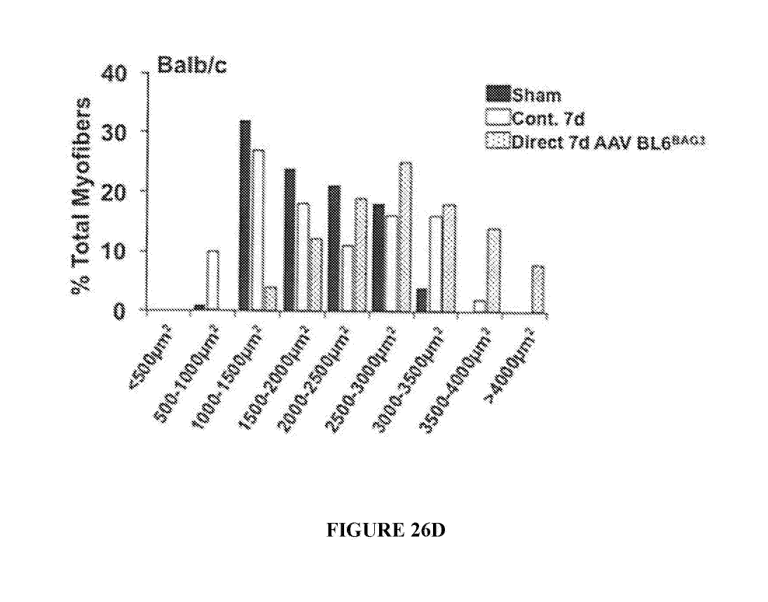

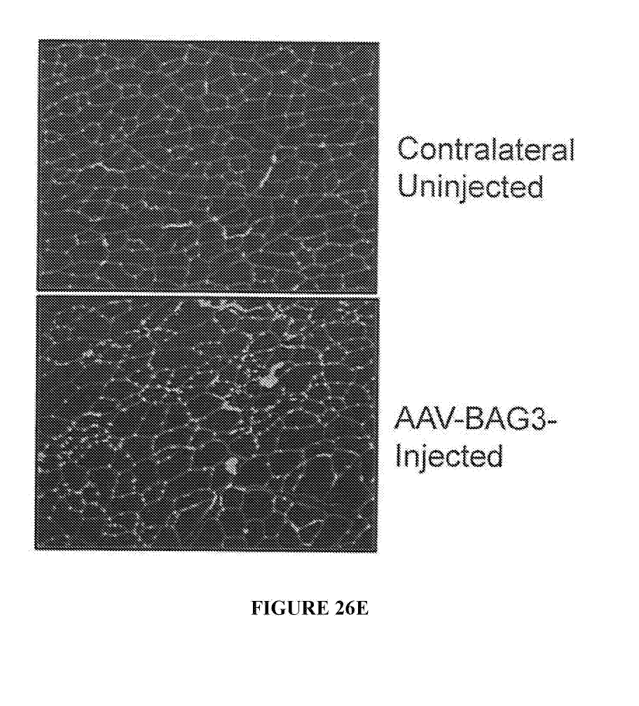

Mice treated with AAV-BAG3^{Ile81} for 7 days without subsequent HLI were also evaluated to see if beneficial effects of BAG3^{Ile81} occurred in resting, non-ischemic muscle. Similar to the effects observed after HU, animals treated with AAV-BAG3^{Ile81} for 7 days and without subsequent ischemia displayed markedly increased muscle fiber cross sectional area, capillary density, and vascular luminal diameter (FIGS. 26A-26E). These findings suggest that pre-treatment of skeletal muscle with BAG3^{Ile81} induces changes in muscle capillary density and vessel diameter along with a myocyte gene expression profile that is protective against ischemia.

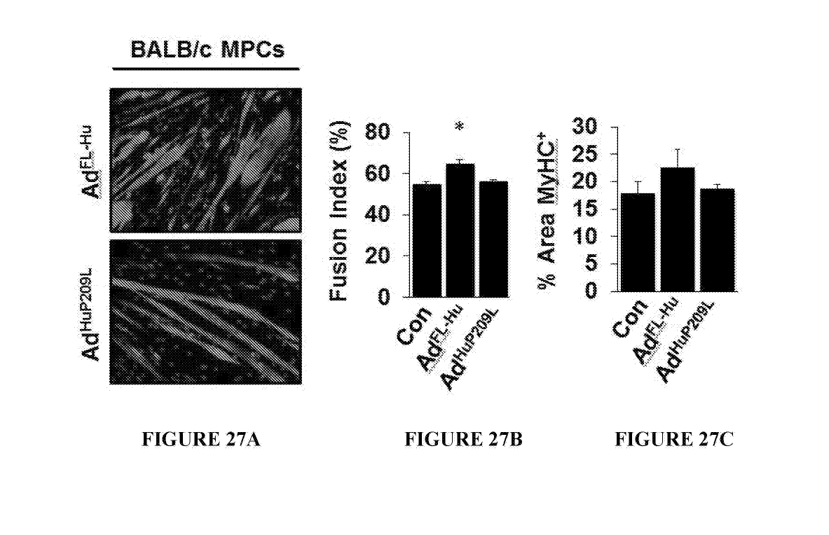

Example 10: Alterations in BAG3 expression or mutations in BAG3 alter its ability to improve muscle cell function. BALB/c primary myoblasts were infected with recombinant adenovirus expressing full-length human (FL-Hu) BAG3 or BAG3 with a known functional mutation that contributes to hereditary myofibrillar and cardiomyocyte myopathy (P209L) and examined for the ability to differentiate (form myotubes, indicated by myosin heavy chain (MyHC)-positive myofibers with multiple nuclei, in red; FIG. 27A). The fusion of myoblasts into myotubes (differentiation; Fusion Index %) and the size of the myotubes (maturation; % Area MyHC+) were both improved by expression of full-length human BAG3 but not P209L (FIGS. 27B-27C).

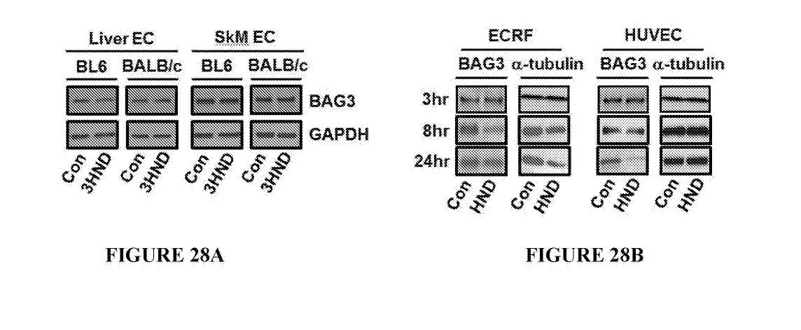

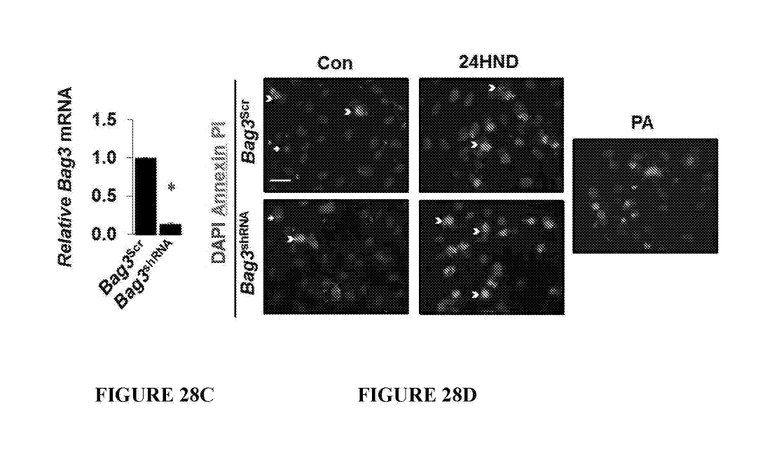

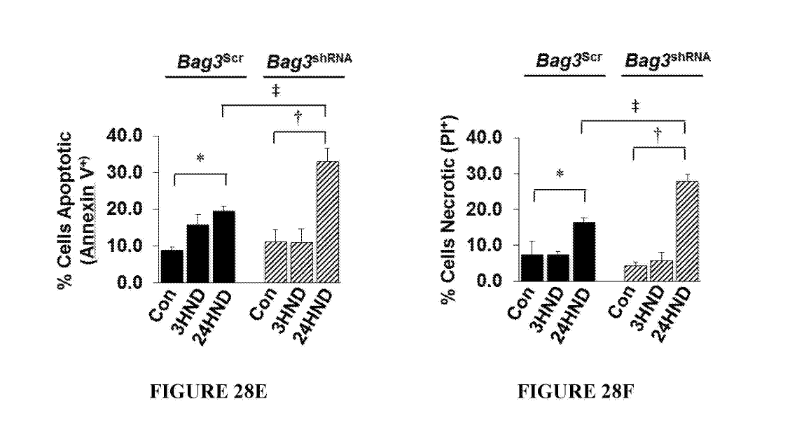

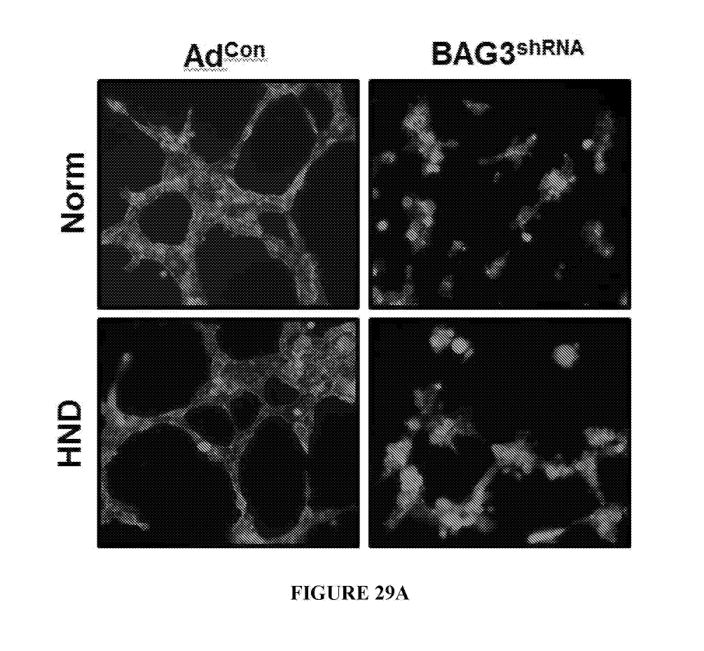

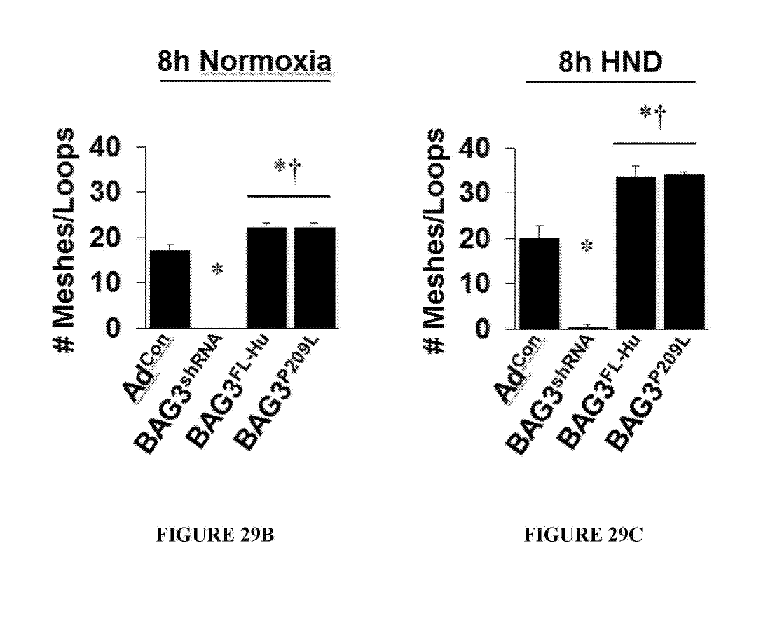

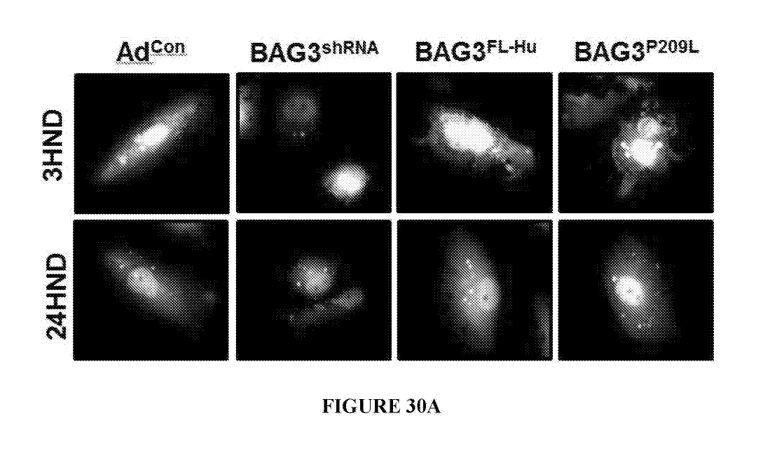

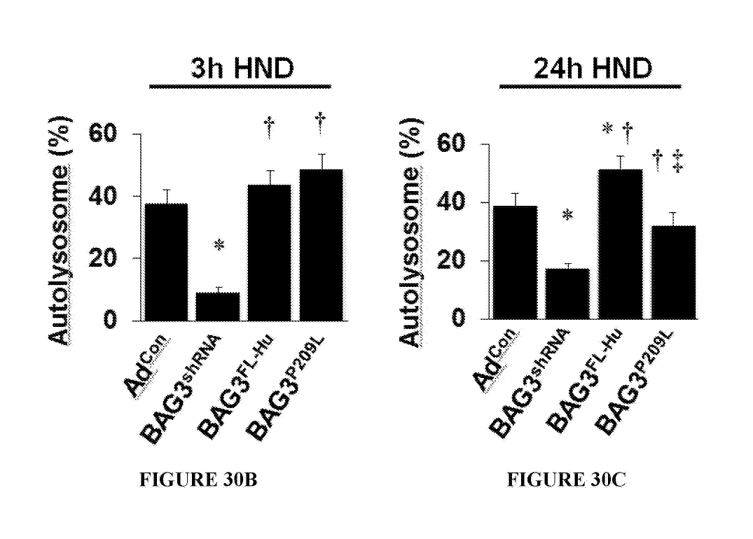

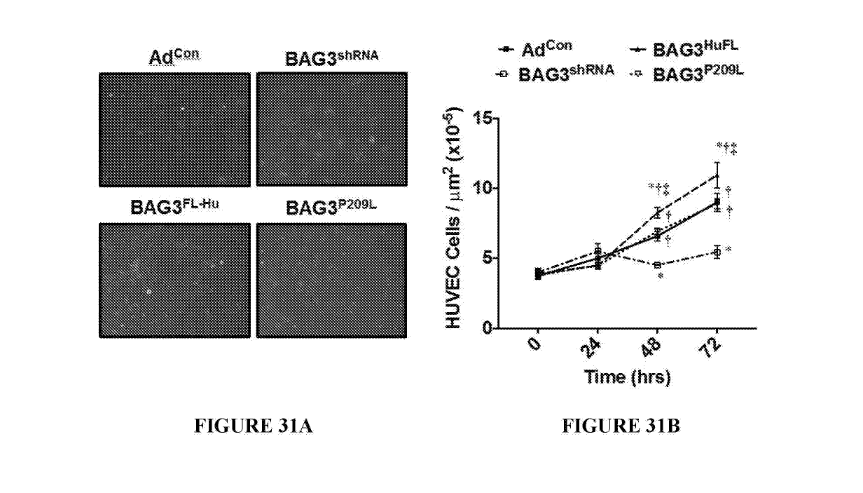

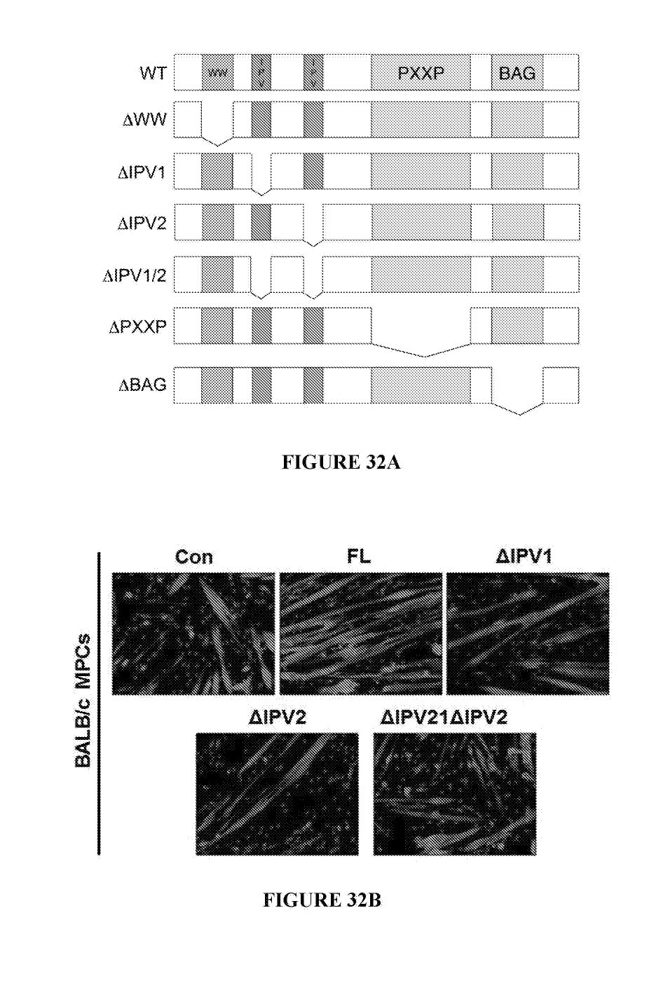

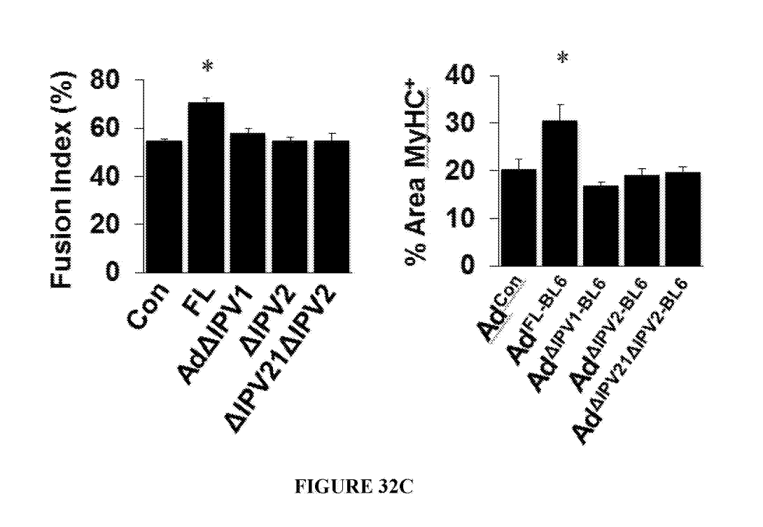

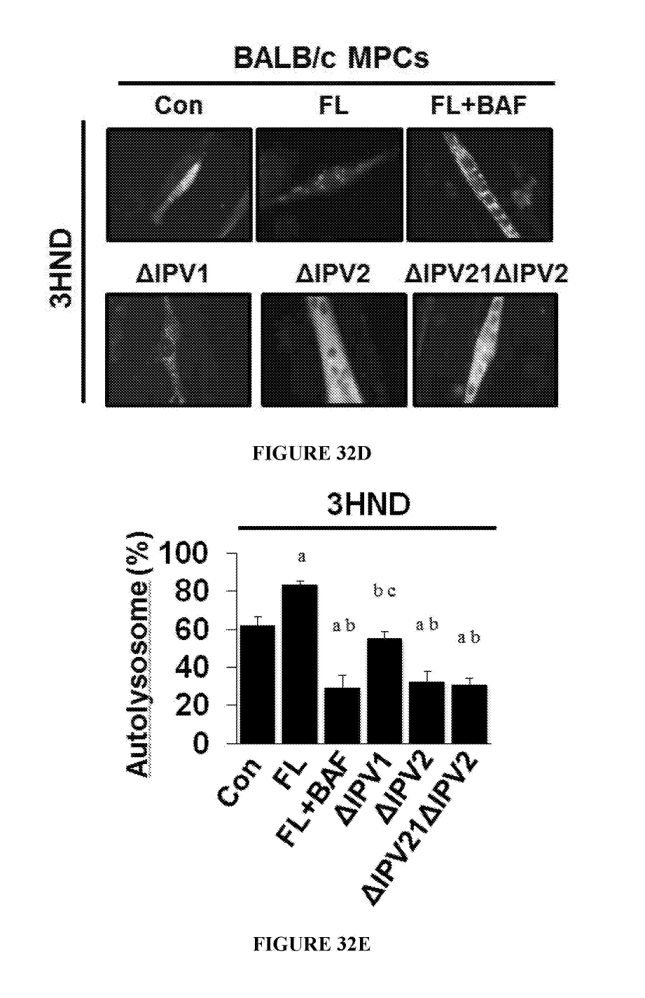

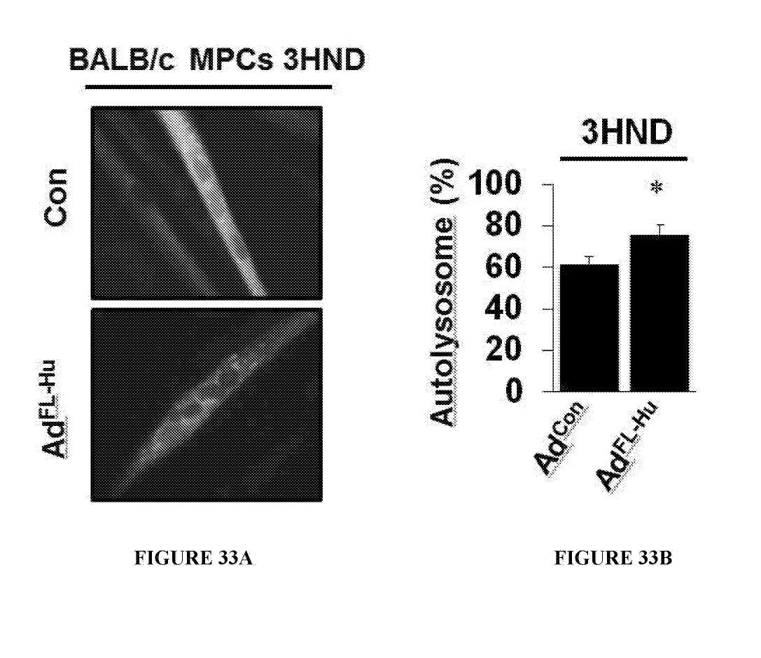

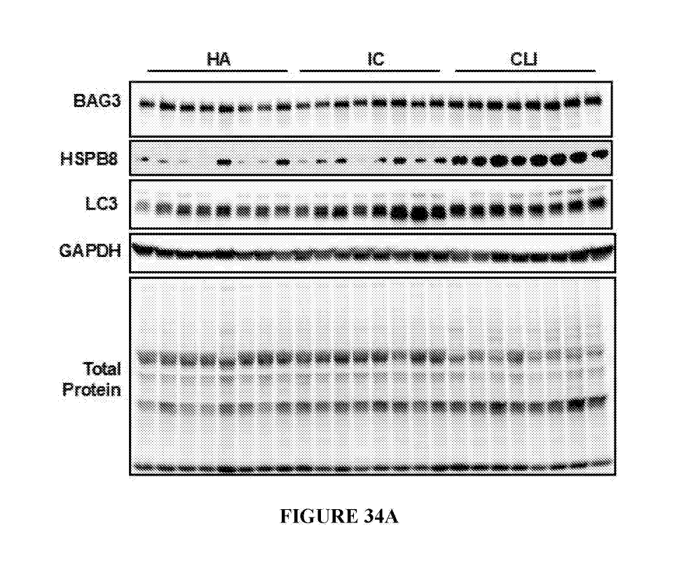

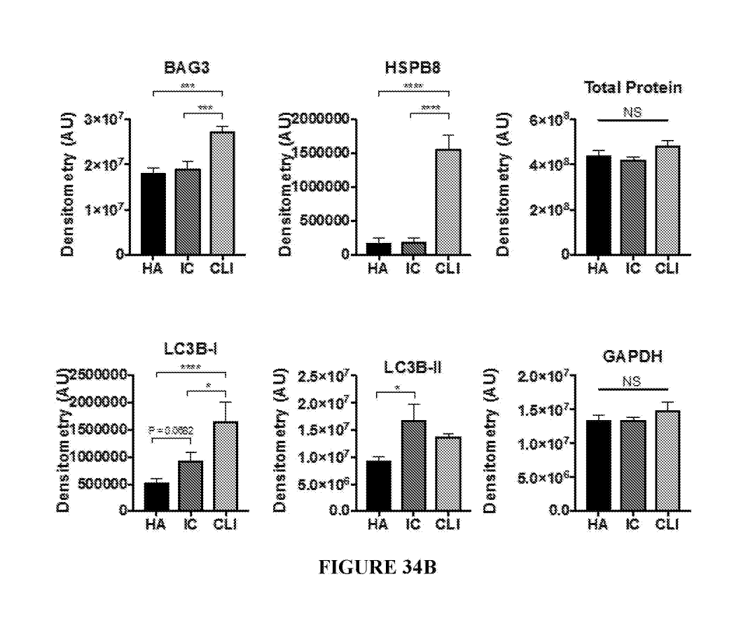

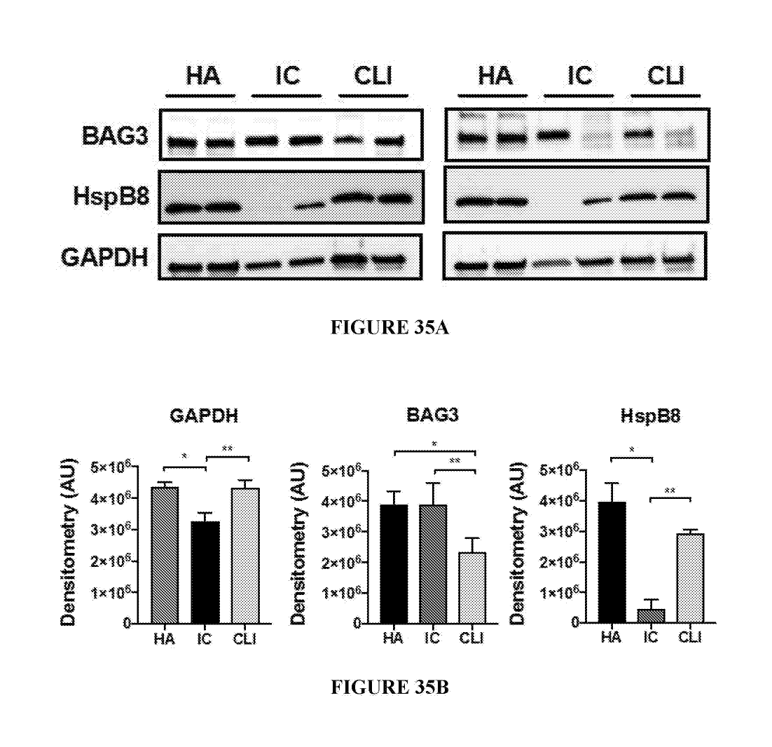

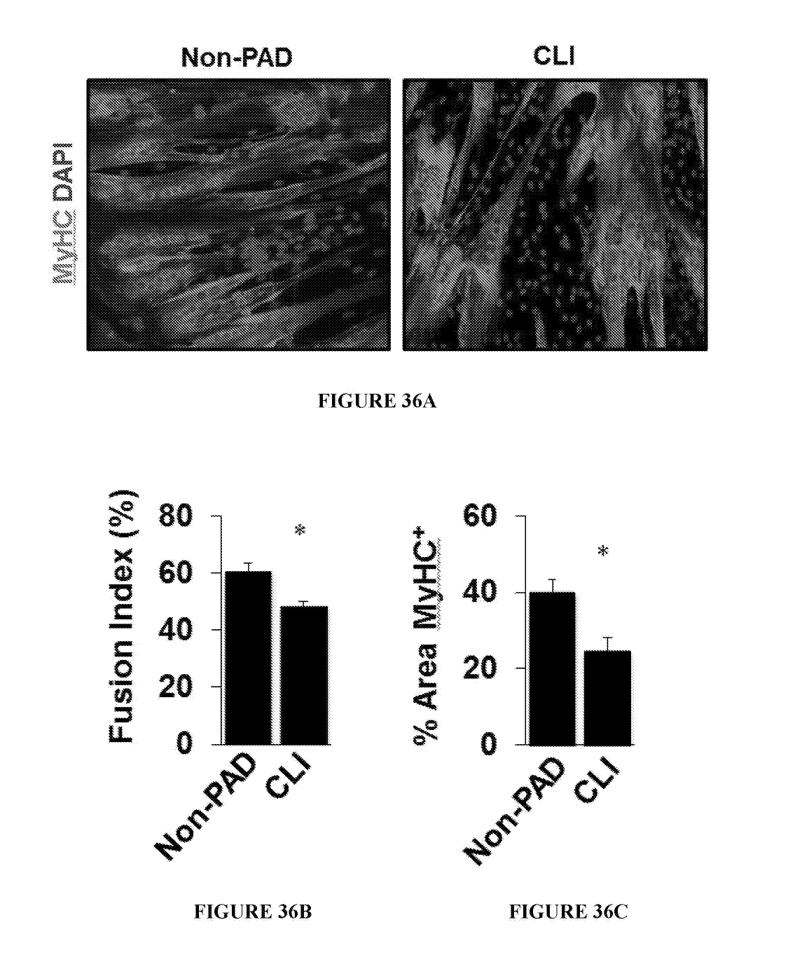

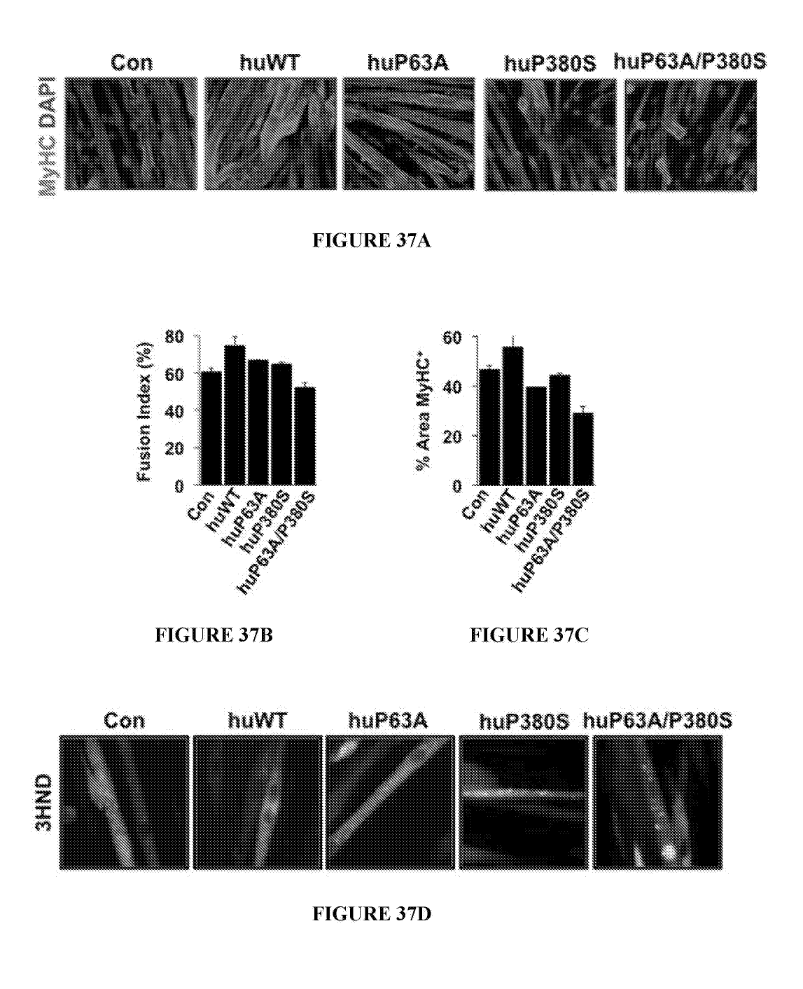

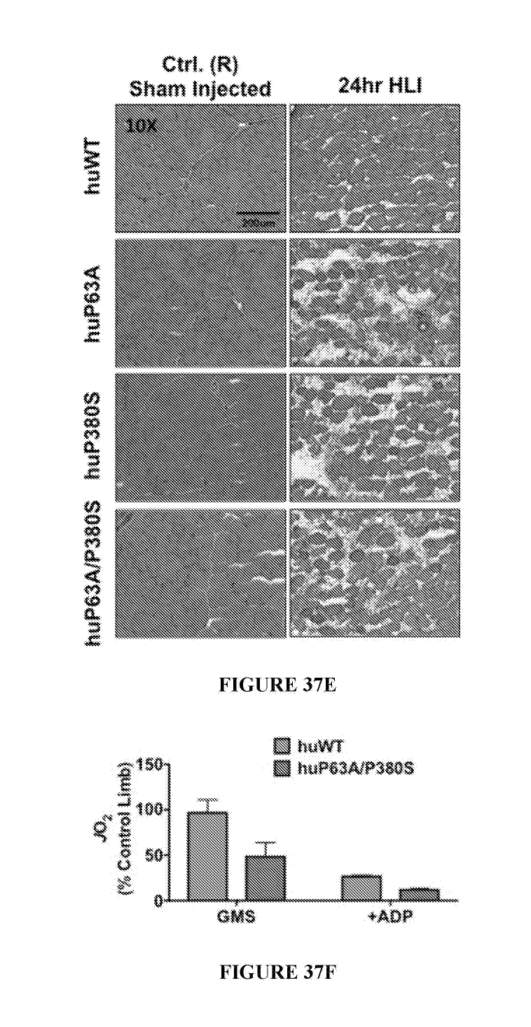

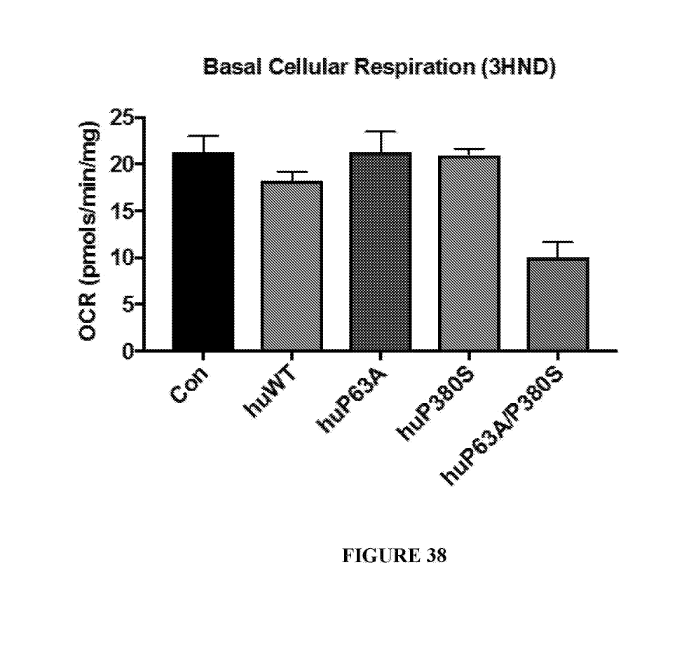

To investigate cell-specific effects of BAG3, isolated primary liver and skeletal muscle endothelial cells (EC) were isolated from BL6 and BALB/c mice and examined BAG3 protein expression, which was stable in abundance in these cells even after 3 h of hypoxia and nutrient deprivation (HND) in vitro compared to control (Con) normoxic cells (FIG. 28A). Similarly, BAG3 protein abundance was relatively unchanged in immortalized ECRF ECs and in primary human umbilical vein endothelial cells (HUVECs) after short (3 h) or intermediate duration (8 h) ischemia, but was reduced after 24 h ischemia in these cells in vitro (FIG. 28B). To elucidate the mechanism of action of BAG3 in endothelial cells, BAG3 was knocked down in HUVECs with an adenovirus encoding a BAG3 shRNA or a control, scrambled (Scr) shRNA (FIG. 28C), and cellular apoptosis (Annexin V staining) and necrosis (propidium iodide staining) was examined after 24 h ischemia (FIG. 28D). Cellular apoptosis and necrosis were increased in control-treated cells after 24 h ischemia and were exacerbated by BAG3 knockdown (FIGS. 28E-28F). Overall, these results demonstrate that BAG3 is important in the prevention of endothelial cell death over extended periods of ischemia.

HUVECs were subjected to adenovirus-mediated BAG3 knockdown (shRNA) or overexpression of full-length human (FL-Hu) BAG3 or BAG3 with a known functional mutation that contributes to hereditary myofibrillar and cardiomyocyte myopathy (P209L) and examined for the ability to form mesh/loop networks (angiogenesis assay) after 8 h of normoxia or hypoxia and nutrient deprivation (HND). Loop formation (FIG. 29A) was visualized by immunofluorescence imaging using phalloidin. Quantification of the number of loops demonstrated that the loss of BAG3 expression prevented angiogenesis in either normoxic or hypoxic conditions, while expression of either full-length human BAG3 or the P209L mutant improved loop formation under both conditions (FIGS. 29B-29C).

To investigate the effects of BAG3 on endothelial cell autophagic flux, HUVECs were co-infected with an RFP-GFP-LC3 reporter adenovirus and either an empty control adenovirus or viruses expressing BAG3shRNA, full-length human (FL-Hu) BAG3, or the human P209L mutant. Autolysosome formation (FIG. 30A) was visualized by immunofluorescence imaging. The loss of BAG3 expression reduced autolysosome formation after both short- (3 h) and long-term (24 h) HND (FIGS. 30B-30C). Full-length human BAG3 or P209L did not alter autolysosome formation after 3 h ischemia, but the two variants had differential effects on autolysosome formation after prolonged (24 h) ischemia, indicating a lack of effectiveness of P209L to improve EC autophagic flux during longer periods of ischemia.

To examine whether endothelial cell proliferation is affected by BAG3 knockdown or mutation, HUVECs were infected with viruses expressing BAG3shRNA, full-length human BAG3, or the human P209L mutant. Proliferation over 72-hours (FIG. 31A) was visualized by imaging cell number after exposure to nuclear dye. The loss of BAG3 expression suppressed HUVEC numbers after 48- and 72-hours of culture (FIG. 31B). Full-length human BAG3 overexpression improved HUVEC proliferation after 48- and 72-hours of culture. There was no effect of BAG3-P209L on HUVEC proliferation, indicating the ability of BAG3 to increase HUVEC proliferation in the absence of a functional mutation.

Example 11: Analysis of the roles of specific BAG3 domains. To elucidate the mechanism of action of BAG3 SNPs on cellular function in ischemia, the effects of BAG3's individual protein interaction domains on endothelial or muscle cell biology were assessed. A series of preliminary experiments were performed in primary BALB/c mouse muscle progenitor cells (MPCs) and HUVECs in vitro using adenoviruses expressing domain-specific deletion and knockdown constructs (FIG. 32A). Overexpression of full-length (FL) BAG3 improved primary myoblast differentiation/fusion (FIGS. 32B-32C), as shown by increased myonuclei within myosin heavy chain (MyHC)-positive myofibers. Consistent with data presented herein, in which a SNP adjacent to the first IPV domain (Ile81Met) disrupted differentiation, the benefits conferred by BAG3-FL were lost when either the IPV1 or IPV2 domain was deleted (FIGS. 32B-32C). Autophagic flux, which is required for myoblast differentiation, was also assessed and shown to be similarly affected by IPV domain deletion. Using the previously described tandem-fluorescence LC3 reporter (tfLC3), a role for the IPV1 and IPV2 domains in myofiber autophagic flux during ischemia (FIGS. 32D-32E) is clearly demonstrated. (BAF, bafilomycin.)

Example 12: Validation of the effectiveness of human BAG3 to rescue a dysfunctional murine autophagy phenotype in muscle cells. It was next examined whether autophagic flux, which is required for myoblast differentiation, is similarly rescued in BALB/c muscle myotubes by overexpression of full-length human (FL-Hu) BAG3. Using the tandem-fluorescence LC3 reporter, a similar effectiveness of human BAG3 to improve BALB/c myofiber autophagic flux during ischemia was demonstrated (FIGS. 33A-33B).

Example 13: Clinical relevance of BAG3 in human peripheral artery disease. To investigate the clinical relevance of BAG3 in human peripheral artery disease, limb gastrocnemius muscle tissues from healthy human adults (age-matched, HA), patients with intermittent claudication (IC), and patients with critical limb ischemia (CLI) were collected and protein expression of BAG3, HspB8, LC3I and II, and GAPDH (FIG. 34A) was examined by western blotting. Densitometry demonstrated a slight increase in BAG3, HspB8, and LC3B proteins in limb tissues from CLI patients (FIG. 34B).

Because the increased expression of BAG3, HspB8, and LC3B-I in patients with CLI was inconsistent with data from mouse studies presented herein, the cellular specificity of BAG3 protein expression was investigated in muscle cells isolated from gastrocnemius muscle tissues from the some of the same subjects described in FIGS. 34A-34B. Primary MPCs were differentiated into myotubes, and protein expression was analyzed by western blotting (FIG. 35A). Densitometry demonstrated a reduction in BAG3 and HspB8 proteins in myotubes from CLI patients (FIG. 35B). Collectively, these data verify a skeletal muscle cell-specific deficit in BAG3 protein expression in CLI patients and suggest that the increased expression seen in the whole tissue blots is driven by other cell types (e.g., inflammatory cells).

Because myotubes from CLI subjects demonstrated reduced BAG3 protein expression, the ability of MPCs from CLI patients to differentiate (form myotubes, indicated by myosin heavy chain-positive myofibers, in red, with multiple nuclei; FIG. 36A) was examined. The fusion of myoblasts into myotubes (differentiation; Fusion Index %) and the size of the myotubes (maturation; % Area MyHC) were both attenuated in CLI patient myotubes (FIGS. 36B-36C).

Example 14: SNPs found to be expressed preferentially in heart failure patients of African-American (AA) descent (hereafter designated BAG3AA-SNP) have dominant inhibitory effects in cardiomyocytes. To test whether these detrimental effects are recapitulated in skeletal muscle and endothelial cells, AAVs encoding several of these human (hu) variants were generated, including P63A and P380S, as well as a P63A/P680S double mutant virus. BALB/c MPCs were infected with these viruses and effects on myoblast fusion, myotube maturation, and autophagy were evaluated (FIG. 37). Staining for myosin heavy chain (MyHC) and DAPI demonstrated that BAG3AA-SNP decreased myoblast fusion and myotube maturation (FIGS. 37A-37C), and co-expression of a tflLC3 reporter demonstrated that BAG3AA-SNP inhibited autophagic flux, as indicated by the presence of green and/or yellow puncta after 3 h of experimental ischemia (FIG. 37D). AAV-mediated overexpression of huWTBAG3, but not huP63A, huP380S, or huP63A/P380S rescued BALB/c muscle morphological abnormalities observed histologically (FIG. 37E) as well as mitochondrial function measured in permeabilized myofibers after 24 h HLI (FIG. 37F).

To investigate further whether these variants' effects also have an impact on mitochondrial function in mice, AAVs were used to express the BAG3AA-SNP in BALB/c mouse myotubes. As shown in FIG. 38, expression of the P63A/P380S double mutant inhibited basal myotube mitochondrial respiration after 3 h HND.

### Materials and Methods

Animals. Experiments were conducted on adult C57/BL6J (n=34), BALB/cJ (n=151), or BALB/c congenic mice containing a 12.06 Mb region of chromosome 7 from C57/BL6J (C.B6-Lsq1-3 or also known as C.B6-Civq1-3; Congenic; n=5) (all ≥10 weeks old), were approved by the East Carolina University, Duke University, or University of Virginia Institutional Animal Care and Use Committees, and conformed to the Guide for the Care and Use of Laboratory Animals published by the US National Institutes of Health. Hindlimb ischemia (HLI), necrosis monitoring, and laser Doppler perfusion monitoring were performed as described previously. The cardiotoxin model of mouse muscle regeneration was performed as described previously using intramuscular injections of Naja nigricollis venom. After systemic adeno-associated virus (AAV) injection, HLI surgeries were modified by leaving the inferior epigastric, lateral circumflex, and superficial epigastric artery collateral branches intact.

MRI. MR imaging was performed on a Bruker 7T (70/30) system (Bruker Biospin, Billerica, Mass., USA). ADC perfusion imaging and magnetic resonance angiography (MRA) for assessment of collateralization were performed with ABLAVAR (Lantheus Medical Imaging, Inc.), a novel blood pool agent, to image vascular volume and collateral vessels.

RNA Isolation and QRT-PCR. Total RNA was extracted using Trizol and was reverse-transcribed using Superscript III Reverse Transcriptase Kits. Real-time PCR was performed using a 7500 Real-Time System (Applied Biosystems, Foster City, Calif.).

Cell Lines and Culture. Murine C2C12 and C3H-10T1/2 cell lines were purchased from ATCC. Immortalized EC-RF24 cells (ECRF) were a gift from Dr. Hans Pannekoek, The University of Amsterdam. Human umbilical vein endothelial cells (HUVECs) were isolated from donor lacental umbilical veins and used prior to passage 6. GP2-293 and 293 cells for adenovirus generation were cultured in DMEM with 10% FBS. Primary murine skeletal myoblasts were isolated as described. Myoblast cell proliferation was assessed by methanol fixation and image analysis. lmmunofluorescence (IF) for myosin heavy chain (MyHC) and nuclei (DAPI) was performed for myotube fusion.

Limb Muscle Morphology and Regeneration. Histological staining was performed according to standard procedures on 8-μm-thick transverse sections of tibialis anterior (TA) muscle. Sections of TA muscle were stained with H&E for the analysis of non-contractile tissue area. Total or embryonic (e)MyHc⁺ myofiber cross sectional area (CSA, μm²) was determined with NIH lmageJ software. Contractile force measurements were performed using single extensor digitorum longus (EDL) muscles, as described previously.

Virus Generation. Pantrophic BAG3 shRNA or GFP control retroviruses were generated by cotransfection of GP2-293 cells with shRNA (SA Biosciences) and envelope (VSVG) plasmids. BAG3 shRNA (sequence derived from TRCN0000293298, Sigma-Aldrich) or scrambled (Scr) control (Sigma-Aldrich) annealed oligos were also ligated into pLK0.1-TRC cloning vector (Addgene, plasmid #10878). The full pLK0.1 shRNA cassette was cloned via In-Fusion (Clontech) into pAdeno-X PRLS Universal System 3 vector (Clontech). mRFP:EGFP:LC3, from plasmid ptfLC3 (Addgene #21074), was cloned via In-Fusion (Clontech) into the pAdeno-X adenoviral vector (Clontech). Adenoviruses were generated by transfection of Adeno-X 293 cells using CalPhos Mammalian Transfection Kit (Clontech). Adeno-Associated Viruses (GFP, BAG3^{Met81}, BAG3^{Ile81}) were generated and purified by column chromatography at the UNC Viral Vector Core Facility.

Autophagic Flux. Autophagic flux was assessed in myotubes using an adenovirus expressing the RFP-GFP-LC3 reporter. Punctate structures with GFP-RFP and/or RFP signals were quantified in more than 120 cells per group, and the degree of autophagosome maturation was expressed as the percent of puncta with red color, as previously described.

Statistical Analysis. Statistical analyses were carried out using StatPlus:mac (v. 2009) statistical analysis software, Vassarstats (vassarstats.net), or Prism 6 (v. 6.0d). No differences in outcome measures between BL6 and BALB/c were observed in contralateral (non-ischemic control) limbs by a priori analysis, therefore contralateral limbs were pooled as non-ischemic controls (Control) for analyses. Non-parametric necrosis score data were presented as proportions and compared using Mann-Whitney U tests. For MR angiography analyses, data were evaluated using Student's t-test. All other data were compared using ANOVA or repeated measures ANOVA with Tukey's post hoc tests or Student's 2-tailed t-test. In all cases, P<0.05 was considered statistically significant and values are presented as means±SE.

Animals. Experiments were conducted on adult C57BL/6 (BL6; N=34), BALB/c (N=151), or BALB/c congenic mice (Congenic, C.B6-Lsq1-3 or also known as C.B6-Civq1-3; N=5) (≥10 weeks old), approved by either the East Carolina University, Duke University, or University of Virginia Animal Care and Use Committees and conformed to the Guide for the Care and Use of Laboratory Animals published by the US National Institutes of Health. Briefly, hindlimb ischemia was performed by anesthetizing mice by intraperitoneal injection of ketamine (90 mg/kg) and xylazine (10 mg/kg) and surgically inducing unilateral hindlimb ischemia with ligation and excision of the fem oral artery from its origin just above the inguinal ligament to its bifurcation at the origin of the saphenous and popliteal arteries. The inferior epigastric, lateral circumflex, and superficial epigastric artery branches were also isolated and ligated. After induction of ischemia semiquantitative necrosis scoring and laser Doppler perfusion monitoring were performed as described below. Ischemia surgeries and necrosis scoring on virus-treated animals were performed in several cohorts of animals from 3 laboratories at 3 institutions by blinded investigators for effect validation. A subset of BL6 and BALB/c animals were subjected to a modified version of hindlimb ischemia as previously described³, where the femoral artery was singularly ligated and transected just inferior to the inguinal ligament and the inferior epigastric, lateral circumflex, and superficial epigastric artery collateral branches were left intact. The cardiotoxin (CTX) model of mouse muscle regeneration was performed as previously described⁴ using 20 μL I.M. injections of 5 μM Naja nigricollis venom into the tibial is anterior (TA) muscle under anesthesia. An equivalent volume sham injection of 1× phosphate buffered saline (PBS) was administered to the muscles of the contralateral hindlimb. The full breakdown of animal usage per experimental group (across all Institutions) is as follows: 1) parental BL6 mice for HLI (N=24); 2) BL6+AAV-BAG3^{Met81} for HLI (N=5); 3) Congenic mice for HLI (N=5); 4) BALB/c mice for HLI (N=15); 5) BALB/c+AAV-GFP for HLI (N=30); 6) BALB/c+AAV-BAG3^{Met81} for HLI (N=21); BALB/c+AAV-BAG3^{Ile81} for HLI (N=22); 7) BALB/c+AAV-GFP for CTX (N=4); 8) BALB/c+AAV-BAG3^{Met81} for CTX (N=4); 9) BALB/c+AAV-BAG3^{Ile81} for CTX (N=4); 10) BL6 for modified HLI (N=5); 11) BALB/c+AAV-GFP for modified HLI (N=5); 12) BALB/c+AAV-BAG3^{Met81} for modified HLI (N=12); 13) BALB/c+AAV-BAG3^{Ile81} for modified HLI (N=10); 14) BALB/c for cell isolations (N=24).

Assessment of tissue necrosis. The extent of necrosis, if any, in ischemic limbs was recorded post-operatively using the previously described semi-quantitative scale: grade 0, no necrosis in ischemic limb; grade I, necrosis limited to toes; grade II, necrosis extending to dorsum pedis; grade III, necrosis extending to crus; and grade IV, necrosis extending to mid-tibia or complete limb necrosis. For limb necrosis, each animal was scored by a blinded investigator at each time point and all scores were assigned across each model by the same blinded investigator.

Laser Doppler perfusion imaging. Limb blood flow was measured using laser Doppler perfusion imaging (LDPI) as previously described with the following modifications. Imaging was performed at a 4 ms/pixel scan rate on animals placed on a 37° C. warming pad in the prone position under ketamine/xylazine anesthesia using a Moor Instruments LD12-High Resolution (830 nM) System (Moor, Axminster, UK). Hindlimb hair was removed with depilatory cream 24 hours prior to initial scanning and hair was removed with a microshaver at all other timepoints. Images were analyzed with the MoorLDI Image Review software. Mice were closely monitored during the postoperative period and flow in the ischemic and contralateral non-ischemic limbs was measured immediately after surgery to verify successful surgery.

Magnetic resonance (MR) imaging. MR imaging was performed on a Bruker 7T (70/30) system (Bruker Biospin, Billerica, Mass., USA) utilizing a quadrature surface receive and volume transmit coil set-up with active decoupling. Animals were anesthetized (induction: 5% isoflurane, maintenance 1.5% isoflurane, with room air mixture) and placed in an MRI-compatible cradle equipped to maintain body temperature constant using warm water circulation. Temperature and respiratory rate were continuously monitored. T2-weighted anatomic images were first acquired using a RARE-based fast spin echo sequence with TR=4200, TE=12, RARE factor 8, 1 mm slice thickness, FOV 2.4 cm, 256×256, with respiratory gating. T2 images are displayed as 3D maximum intensity projection images for correlation to MR angiography (MRA). MRA was performed using a contrast-enhanced T1-weighted time-of-flight sequence in the coronal plane with 2D FLASH, using TE/TR+3.8/15 ms, FOV=4 cm×4 cm, matrix of 256×256, and 120 slices. Vascular contrast was enhanced utilizing intravenous gadofosveset trisodium (ABLAVAR, Lantheus Medical Imaging, Inc.), at 0.03 mmol/Kg. This agent is clinically approved for optimization of blood pool imaging by virtue of specific binding to serum albumin. Perfusion maps were then generated using a double spin-echo planar pulse sequence using pairs of bipolar gradients at specific predetermined signs in each of three orthogonal directions. The combination of gradient directions allows cancellation of all off-diagonal tensor elements, enabling measurement of the diffusion tensor trace, and therefore providing unambiguous and rotationally invariant ADC values. Four b values (b=0, 50.0, 100, and 200) were acquired, with a matrix size of 128×128, slice thickness 1.0 mm. Volume images (one for each b value) were created from raw DICOM images. For voxels within the 128×128×15 matrix with a signal value above 2000, the apparent diffusion coefficient (ADC) at each voxel was calculated using an exponential moving fit by the following method: ADC=In [S(b=b₁)−S(b=b₂)/b₂−b₁. B1 and b2 values of 100 and 200, respectively, are sensitive to blood flow apparent diffusion changes in small arteries and capillaries. ADC maps were generated using mono-exponential fitting as above, and T2 images were zero-filled to 256×256 prior to analysis. Parametric images were analyzed in anatomic regions of interest (ROIs) using Bruker Paravision software and offline using Osirix software.

Muscle contractile force measurements. Contractile force measurements were performed using extensor digitorum longus (EDL) muscles as previously described. In brief, single EDL muscles were surgically excised with ligatures at each tendon (5-O silk suture) and mounted in a bath between a fixed post and a force transducer (Aurora 300B-LR) operated in isometric mode. The muscle was maintained in modified Krebs buffer solution (PSS; pH 7.2) containing 115 mM NaCl, 2.5 mM KCl, 1.8 mM CaCl², 2.15 mM Na₂HPO₄, and 0.85 mM NaH₂PO₄, and maintained at 25° C. under aeration with 95% O₂-5% CO₂ throughout the experiment. Resting tension and muscle length were iteratively adjusted for each muscle to obtain the optimal twitch force, and a supramaximal stimulation current of 600 mA was used for stimulation. After a 5 min equilibration, isometric tension was evaluated by 200 ms trains of pulses delivered at 10, 20, 40, 60, 80, 100, and 120 Hz. Length was determined with a digital microcaliper. After the experimental protocol, muscles were trimmed proximal to the suture connections, excess moisture was removed, and the muscle was weighed. The cross-sectional area for each muscle was determined by dividing the mass of the muscle (g) by the product of its length (Lo, mm) and the density of muscle (1.06 g/cm³) and was expressed as millimeters squared (mm²). Muscle output was then expressed as specific force (N/cm²) determined by dividing the tension (N) by the muscle cross-sectional area⁷.

RNA Isolation and RT-PCR. Total RNA was extracted using Trizol-phenol/chloroform isolation procedures and was reverse-transcribed using Superscript III Reverse Transcriptase and random primers (lnvitrogen Inc.). Real-time PCR was performed using a 7500 Real-Time PCR System (Applied Biosystems, Foster City, Calif.). Relative quantification of Bag3 mRNA levels was determined using the comparative threshold cycle (ΔΔ CT) method using FAM TaqMan® Gene Expression Assays (Applied Biosystems) specific to the given gene run in complex (multiplex) with a VIC-labeled GAPDH control primer.

Primary Antibodies and Materials. The following commercial antibodies were used: FLAG, LC3b, ATG7, Beclin, HspB8, SQSTM1/p62 (Cell Signaling), BAG3 (Polyclonal, Imgenex), GAPDH (Novus Biologicals), tubulin (Santa Cruz), CD31 (Abd Serotec MCA-1364), SMA (DAKO, 1A4). For immunofluorescence: Dystrophin (Thermo Scientific RB-9024), and Pax7, eMyHC (F1.652), and Dystrophin (MANDYS1 3B7) (all from Developmental Studies Hybridoma Bank, Iowa City, Iowa), CD31 (Abd Serotec MCA-1364), and SMA (DAKO, 1A4). The TSA amplification kit (#24, with HRP-streptavidin and Alexa Fluor 568 tyramide, Molecular Probes) was utilized exclusively for Pax7 immunofluorescence.

Histological analysis. Skeletal muscle morphology, vessel density, and markers of muscle regeneration were assessed by standard light microscopy and immunofluorescence microscopy as previously described³. Eight-μm-thick transverse sections were cut from mouse TA muscle, frozen in liquid nitrogen-cooled isopentane in optimum cutting temperature (OCT) medium. Sections were allowed to come to room temperature and were either stained with hematoxylin and eosin using standard methods or fixed and permeabilized with ice-cold acetone for 10 min at 4° C. Sections from TA muscle samples were stained with H&E, and digital images were obtained at ×10 magnification for the analysis of non-contractile tissue expansion. A 528 (22×24)-point grid was overlain on 3 images from each animal, and points were analyzed for occurrence on myofibers or outside of myofibers and expressed as the percentage of non-myofiber area in HLI muscle as an indication of muscle myofiber reformation and hypertrophy. For the analysis of myofiber integrity, approximately 300 individual fibers visualized by immunofluorescence labeling for dystrophin and DAPI were quantified for disrupted dystrophin staining (>50% of fiber membrane area dystrophin negative) and expressed as the percentage of total fibers with intact dystrophin immunostaining (% intact TA myofibers). Total or eMyHC⁺ myofiber cross sectional area (CSA, μm²) was determined using ×10 images by analyzing approximately 300 individual fibers with NIH lmageJ image analysis software. Images were also utilized for the localization of centralized myofiber nuclei, expressed as a percentage of total myofibers with centralized nuclei.

Immunofluorescence (IF). IF was used for the visualization of muscle morphology, vessel density, and muscle regeneration. 8-μm-thick transverse sections were cut from TA muscle frozen in liquid nitrogen cooled isopentane in OCT. Sections were allowed to come to room temperature and fixed/permeabilized with ice-cold acetone for 10 min at 4° C. Fixed sections were rehydrated in 1× PBS before blocking in 5% normal goat serum (Sigma) in 1× PBS at RT for 45 min. Slides were then incubated overnight at 4° C. in a primary antibody solution. Slides were then washed 3× in 1× PBS at RT and incubated for 1 h at RT in the dark in a secondary solution containing a 1:250 dilution of Alexa Fluor 488-, 568-, or 633-conjugated secondary antibodies in blocking solution. Sections were then washed in the dark 3× for 5 min each with 1× PBS at RT, and slipcovers were mounted using Vectashield HardSet Mounting Medium with DAPI (Vector Labs H-1500). Images were captured using a Zeiss Axio Observer Inverted Laser Scanning Microscope (LSM) 510 utilizing the Zeiss LSM 510 software (v. 4.2) and analyzed by a blinded investigator using lmageJ software (NIH, v. 1.49v). Vessel density was quantified as the number of CD31⁺ cells per μm² of muscle analyzed. The density of CD31⁺ vessels was quantified as an indicator of capillary density changes in the distal limb muscle and represents capillary regression or angiogenesis. Pax7 staining was performed as previously described. Sections were then washed 3× for 5 min in the dark with 1× PBS at RT and slipcovers were mounted using Vectashield HardSet Mounting Medium with DAPI (Vector Labs H-1500). Images were captured using a Zeiss Axio Observer Inverted Laser Scanning Microscope (LSM) 510 utilizing the Zeiss LSM 510 version 4.2 software and analyzed using NIH lmageJ software as follows: CD31+, SMA+, PAX7+, and eMyHC+ labeled cells were counted and expressed as the ratio of positively stained cells/μm² of TA muscle analyzed. Representative immunofluorescence images from animals infected with AAV-GFP viruses were pseudo-colored green for visualization.

SDS-PAGE, western blotting (WB), and immunoprecipitation (IP). SDS-PAGE and WB were performed according to standard methods. Frozen muscles were homogenized in ice-cold RIPA lysis buffer containing protease and phosphatase inhibitors. Protein concentrations were determined using a BCA protein assay (Pierce, ThermoFisher #23225). Proteins were then separated by SDS-PAGE (Mini-Protean TGX, Bio-Rad #4561093) with equal amounts of total protein loaded per well. For IP, total protein lysates from limb tissues or cell lysates were generated in lysis buffer supplemented with protease and phosphatase inhibitor tablets (Complete PI, PhosSTOP, Roche USA) and allowed to rotate with monoclonal Anti-FLAG Affinity Gel (Sigma, A2220) or BAG3 primary antibody o/n at 4° C.

Cell Lines and Culture. Murine C2C12 and C3H-10T 1/2 cell lines were purchased from A TCC and cultured as per the manufacturer's recommendations. Differentiation was stimulated by serum withdrawal in differentiation medium (DM: DMEM supplemented with 2% horse serum, 1% penicillin/streptomycin, 0.2% amphoteric in B, and 0.01% human insulin/transferrin/selenium). To evaluate the effects of ischemia/hypoxia in skeletal muscle cells in vitro, a model of cellular hypoxia was established in which cells are subjected to 0% O₂ and deprived of nutrients in Hanks' balanced salt solution (HBSS) to mimic the local environment resulting from severe ischemia in PAD (referred to hereafter as hypoxia+nutrient deprivation, HND). GP2-293 cells for pantrophic retrovirus generation were cultured at 37° C. and 5% CO2 in DMEM with 10% FBS. Transfections were done with Lipofectamine-Plus reagent (Invitrogen).

Primary Myoblast Isolation and Culture. Primary murine muscle precursor cells (mouse myoblasts) derived from hindlimb muscles were prepared as previously described. Briefly, peripheral skeletal muscle was dissected from 6-week old female mice using sterile technique, trimmed of connective tissue, and placed in 10-cm dishes containing ice cold sterile PBS. Organs were then transferred to separate 10-cm dishes containing 5mL of pre-warmed MPC isolation medium (IM: DMEM with 4.5 g/L glucose, supplemented with 1% Penicillin/Streptomycin/Amphotericin B) and any remaining connective tissue was trimmed. Organs were then transferred to a third 10-cm dish containing 5 mL of cold MPC IM, transported to the sterile culture hood, and minced for 2 minutes (per plate) using sterile razor blades. The minced slurry was transferred to 15 mL tubes, 5 mL additional MPC IM was added, tubes were inverted several times and centrifuged at 4° C. for 3 min at 700×g to remove contaminants. The MPC IM was subsequently aspirated and the pellet was resuspended in 10 mL of MPC IM and inverted 5-10× to loosen the pellet and mix bet ore decanting into a 10-cm culture dish. Tubes were subsequently rinsed with 8 mL MPC IM to ensure all tissue was removed, and 2 ml of 1% pronase (Calbiochem #53702) was added to a final concentration of 0.1%. A sterile, low-profile magnetic stir bar was added, and dishes were stirred at low rpm on a magnetic stir plate at 37° C. and 5% CO2 for 1 hr. The digested tissue slurry was then transferred to 50 mL conical tubes and centrifuged for 4 min at 800×g at RT. The supernatant was aspirated and the digested pellet was resuspended in 10 mL MPC purification medium (PM: DMEM with 4.5 g/L glucose, supplemented with 10% fetal bovine serum and 1% Penicillin/Streptomycin/Amphotericin B). The suspension was then triturated approximately 20× through a blunt end pipetting needle attached to a sterile 30 cc syringe. The suspension was then passed through a 100 μm disposable Steriflip vacuum filter into a 50 mL tube, including 3 successive 8 mL washes of the sieve with pre-warmed PM, and subsequently centrifuged at RT 5 min at 1000×g. The cell pellet was then resuspended in 1 mL FBS before addition to primary M PC growth medium (GM: Ham's F10, supplemented with 20% FBS and 1% Penicillin/Streptomycin/Amphotericin B, and supplemented immediately prior to use with 5 ng/mL basic FGF). Cells were plated on collagen-coated T150 flasks, allowed to adhere and proliferate for 3-days, and subsequently trypsinized with 0.25% Trypsin/EDTA and pre-plated at 37° C. and 5% CO2 for 1 hr on an uncoated T150 flask to allow for fibroblast removal. The supernatant containing the MPCs was removed and centrifuged at 800×g for 5 min at RT prior to re-plating in MPC GM on collagen-coated T150 flasks. After reaching approximately 70% confluence, MPCs were then plated in pre-warmed GM in either T75 flasks or standard 12-well culture plates coated with entactin/collagen/laminin and allowed to reach approximately 90% confluence. Confluent MPCs were then rinsed once in sterile PBS and switched to OM for myotube formation. OM was changed every 24 hours. Cell purity of myoblasts was verified by immunofluorescence staining for MyoD and DAPI followed by counting the number of MyoD-stained cells as a percentage of total nuclei. Purity of myotubes was also analyzed by immunofluorescence staining of myosin heavy chain (MyHC) and DA P1 after differentiation into myotubes.

Proliferation, apoptosis, and myotube fusion index assays. Muscle myoblast cell proliferation was assessed by plating approximately 50,000 strain-specific and/or pre-infected (GFP, BAG3^{Met81}, or BAG3^{Ile81} AAVs: 1×10⁹ AVP) cells on 6-well plates coated with entactin/collagen/laminin (ECL). Wells were washed with phosphate-buffered saline (PBS), fixed with 100% methanol for 5 min, and left to air dry for 10 min. All experimental wells were then simultaneously stained with hematoxylin for 5-minutes and rinsed 3× in dH₂O. Cell images were obtained via phase contrast at ×10 magnification on an inverted microscope camera system. Total image cell counts were quantified from at least 4 random fields, a number chosen by determination of no additional change in standard deviation, by a blinded investigator. Muscle proliferation numbers were then normalized by treatment to the 0-hour (post-plating) counts to give fold population doubling values. Cellular apoptosis/necrosis was quantified using ApoAlert Annexin V kit (Clontech). Cells were stained with Annexin V-FITC, propidium iodide and DAPI and assessed under standard fluorescent microscopy. lmmunofluorescence for myosin heavy chain (MyHC) and nuclei (DAPI) was performed for myotube fusion analysis as previously described¹⁹. Approximately 100,000 cells per treatment/strain were plated on 12-well plates coated with ECL, allowed to reach 50-60% confluence in primary GM, and infected with either control (GFP), BAG3^{Met81}, or BAG3^{Ile81} AAVs (2×10⁹ AVP) for 24 hrs in DM. DM was then changed every 24 hours. Cells were washed with phosphate-buffered saline (PBS), fixed with 100% methanol for 5 min, left to air dry for 10 min, and immunofluorescently labeled with anti-MyHC. Images were captured using a Life Technologies EVOS auto FL wide field fluorescence microscope (Thermo Fisher) and analyzed by a blinded investigator using lmageJ (NIH, v1.49). Each well was photographed in four randomly selected regions. The number of myonuclei and the total number of nuclei were scored and the fusion index was calculated as the percentage of total nuclei incorporated in myotubes. Each experiment included at least 3 technical replicates and each biological experiment was replicated at least 3 times.

Virus Generation. Pantrophic BAG3 shRNA or GFP control retroviruses were generated by cotransfection of GP2-293 cells with shRNA plasmids (SABiosciences) and envelope plasmid (VSVG). BAG3 shRNA (sequence derived from TRCN0000293298, Sigma-Aldrich) or scrambled (scr) Control (Sigma-Aldrich) annealed oligos were also ligated into pLKO.1-TRC cloning vector (Addgene, plasmid #10878). The full pLKO.1 shRNA cassette was cloned via In-Fusion (Clontech) into pAdeno-X PRLS Universal System 3 vector (Clontech). The insert, mRFP:EGFP:LC3 from plasmid ptfLC3 (Addgene #21074), was cloned via In-Fusion (Clontech) into the Adeno-X adenoviral vector (Clontech). Adenoviruses were generated by transfection of Adeno-X 293 cells using CalPhos Mammalian Transfection Kit (Clontech). pCMV5 containing C-terminal FLAG-tagged coding regions of either BALB/c (Met81) or BL6 (Ile81)-specific mouse Bag3 were moved into pTR-transgene AAV vectors in combination with XX680 for virus generation. Adeno-associated viruses (GFP, BAG3^{Met81}, BAG3^{Ile81}) were generated using mouse strain-specific constructs in suspension HEK293 cells and purified by column chromatography at the UNC Viral Vector Core Facility. AAV viruses were injected in vivo either 1) IM into the TA and medial and lateral gastrocnemius hindlimb muscles (1×10¹⁰ AVP/injection site) and allowed to express for 7 days prior to HLI; or 2) systemically (retro-orbitally; 1×10¹¹ AVP/injection) and allowed to express for 21 days prior to HLI, or they were used in vitro (1×10⁹ AVP). All intramuscular or systemic (retro-orbital) virus-injected animals (regardless of heterogeneity or lack of expression) were included for analysis.

Autophagic Flux. Autophagic flux was assessed in myoblasts and myotubes using an adenovirus expressing the RFP-GFP-LC3 reporter. Sub-confluent myoblasts in a 12-well plate were infected with RFP-GFP-LC3 and BAG3 viruses for 8 hours in low serum medium and subjected to control or experimental ischemia conditions approximately 48-hours later. Confluent primary BALB/c myoblasts in a 12-well plate were also infected with RFP-GFP-LC3 and BAG3 viruses overnight at the time of transition to low serum differentiation medium and then allowed to differentiate for 120 hrs. Images were captured using a Life Technologies Evos auto FL wide field fluorescence microscope (Thermo Fisher) and analyzed by a blinded investigator. Punctate structures with GFP-RFP and/or RFP signals were quantified in more than 120 cells per group, and the degree of autophagosome maturation was expressed as the percent of puncta with red color, as previously described.

Statistical Analysis. Statistical analyses were carried out using StatPlus:mac (v. 2009) statistical analysis software, Vassarstats (vassarstats.net) or Prism 6 (v. 6.0d). No differences in outcome measures between BL6 and BALB/c were observed in contralateral (non-ischemic control) limbs by a priori analysis, therefore contralateral limbs were pooled as non-ischemic controls (Control) for analyses. Non-parametric necrosis score data are presented as proportions and compared using Mann-Whitney U tests. For MRA analyses, data were evaluated using Student's t-test where appropriate. Post hoc tests with ANOVA were performed using Bonferroni's method. All other data were compared using ANOVA or repeated measures ANOVA with Tukey's post hoc tests or Student's 2-tailed t-test. In all cases, P<0.05 was considered statistically significant and values are presented as means±SEM.

Various features and advantages of the invention are set forth in the following claims.

What is claimed is:

1. A method of treating ischemic injury in a subject, the method comprising:
administering a polynucleotide encoding a Bcl2-associated athanogene-3 (BAG3) polypeptide to the subject, wherein the BAG3 polypeptide encoded by the polynucleotide is represented by SEQ ID NO:2; and
treating at least one symptom associated with the ischemic injury in the subject.

2. The method of claim 1, wherein the BAG3 polypeptide encoded by the polynucleotide comprises an isoleucine at amino acid position 79.

3. The method of claim 2, wherein the BAG3 polypeptide comprises at least one amino acid substitution in addition to the isoleucine at amino acid position 79.

4. The method of any of claims 1 to 3, wherein the polynucleotide encoding the BAG3 polypeptide is operably coupled to a targeting vector capable of causing the expression of the BAG3 polypeptide in at least one of a muscle cell, fibroblast, stem cell, pericyte, and endothelial cell.

5. The method of claim 4, wherein the targeting vector comprises an adeno-associated virus (AAV).

6. The method of claim any of claims 1 to 5, wherein the subject has at least one of the following single nucleotide polymorphisms:
(i) a leucine at amino acid position 209;
(ii) an alanine at amino acid position 63;
(iii) a serine at amino acid position 380; and
wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury in the subject.

7. The method of any of claims 1 to 6, wherein the ischemic injury comprises myopathy or vascular deficiency.

8. The method of any of claims 1 to 7, wherein the ischemic injury is caused by one or more of peripheral artery disease comprising intermittent claudication or critical limb ischemia, muscular dystrophy, myofibrillar myopathy, degenerative myopathies, glycogen storage diseases, trauma, renal disease, atrial fibrillation, COPD, coronary artery disease, morbid obesity, cachexia, congestive heart failure, myocardial infarction, and diabetes mellitus.

9. The method of any of claims 1 to 8, wherein at least one symptom is necrosis, and wherein administering the polynucleotide encoding the BAG3 polypeptide treats the ischemic injury in the subject by reducing necrosis.

10. The method of any of claims 1 to 8, wherein at least one symptom is myopathy, and wherein administering the polynucleotide encoding the BAG3 polypeptide treats the ischemic injury in the subject by reducing the myopathy.

11. The method of any of claims 1 to 10, wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury by increasing one or more of muscle fiber cross-sectional area, capillary density, muscle function, muscle regeneration, stem cell activity, vascular density, and vascular luminal diameter.

12. The method of any of claims 1 to 11, wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury by causing one or more of increase in myotube diameter, myotube phenotype, contractile function, an increase in stem cell or satellite cell activity/myogenesis, an increase in mitochondrial number or respiratory function, an increase in autophagic flux, and decreased DNA fragmentation.

13. The method of any of claims 1 to 12, wherein administering the polynucleotide encoding the BAG3 polypeptide represented by SEQ ID NO:2 and/or the polynucleotide encoding the BAG3 polypeptide comprising an isoleucine at amino acid position 79 treats the at least one symptom associated with the ischemic injury by causing one or more of increased expression of vascular endothelial growth factor (VEGF), neuropilin (Nrp-1), vascular endothelial growth factor receptor 1 (Flt), vascular endothelial growth factor receptor 2 (Flk), myogenin, myoD, Tmem8c (myomaker) and muscle RING-finger protein 1 (MuRF-1), and decreased in expression of myostatin.

14. The method of any of claims 1 to 13, wherein administering the polynucleotide encoding the BAG3 polypeptide represented by SEQ ID NO:2 and/or the polynucleotide encoding the BAG3 polypeptide comprising an isoleucine at amino acid position 79 to the subject comprises one or more of intramuscular injection, percutaneous injection, intraperitoneal injection, intravenous injection, and oral consumption.

15. The method of any of claims 1 to 14, wherein administering the polynucleotide encoding the BAG3 polypeptide represented by SEQ ID NO:2 and/or the polynucleotide encoding the BAG3 polypeptide comprising an isoleucine at amino acid position 79 to the subject comprises two or more separate injections.

16. A method of preventing ischemic injury in a subject, the method comprising:
administering a polynucleotide encoding a Bcl2-associated athanogene-3 (BAG3) polypeptide to the subject, wherein the BAG3 polypeptide encoded by the polynucleotide is represented by SEQ ID NO:2; and
preventing the onset of at least one symptom associated with the ischemic injury in the subject.

17. The method of claim 16, wherein the BAG3 polypeptide encoded by the polynucleotide comprises an isoleucine at amino acid position 79.

18. The method of claim 17, wherein the BAG3 polypeptide comprises at least one amino acid substitution in addition to the isoleucine at amino acid position 79.

19. The method of any of claims 16 to 18, wherein the polynucleotide encoding the BAG3 polypeptide is operably coupled to a targeting vector capable of causing the expression of the BAG3 polypeptide in at least one of a muscle cell, fibroblast, stem cell, pericyte, and endothelial cell.

20. The method of claim any of claims 16 to 19, wherein the subject has at least one of the following single nucleotide polymorphisms:
(i) a leucine at amino acid position 209;
(ii) an alanine at amino acid position 63;
(iii) a serine at amino acid position 380; and
wherein administering the polynucleotide encoding the BAG3 polypeptide represented by SEQ ID NO:2 and/or the polynucleotide encoding the BAG3 polypeptide comprising an isoleucine at amino acid position 79 prevents the onset of the at least one symptom associated with the ischemic injury in the subject.

21. A pharmaceutical composition for treating or preventing ischemic injury in a subject, the composition comprising:
a polynucleotide encoding a Bcl2-associated athanogene-3 (BAG3) polypeptide, wherein the BAG3 polypeptide encoded by the polynucleotide is represented by SEQ ID NO:2; and
a pharmaceutically acceptable excipient.

22. The composition of claim 21, wherein the BAG3 polypeptide encoded by the polynucleotide comprises an isoleucine at amino acid position 79.

23. The composition of claim 22, wherein the BAG3 polypeptide comprises at least one amino acid substitution in addition to the isoleucine at amino acid position 79.

24. The composition of any of claims 21 to 23, wherein the polynucleotide encoding the BAG3 polypeptide is operably coupled to a targeting vector capable of causing the expression of the BAG3 polypeptide in at least one of a muscle cell, fibroblast, stem cell, pericyte, and endothelial cell.

25. The composition of any of claims 21 to 24, wherein the composition is formulated for administration to a subject by one or more of intramuscular injection, percutaneous injection, intraperitoneal injection, ingestion, and intravenous injection.

26. A kit comprising:
a pharmaceutical composition comprising a polynucleotide encoding a Bcl2-associated athanogene-3 (BAG3) polypeptide, wherein the BAG3 polypeptide encoded by the polynucleotide is represented by SEQ ID NO:2; and
a delivery device for administering the pharmaceutical composition to a subject.

27. The kit of claim 26, wherein the BAG3 polypeptide encoded by the polynucleotide comprises an isoleucine at amino acid position 79.

28. The kit of claim 27, wherein the BAG3 polypeptide comprises at least one amino acid substitution in addition to the isoleucine at amino acid position 79.
